(19) United States
(12) Patent Application Publication
(10) Pub. No.: US 2026/0337161 A1
(43) Pub. Date: Oct. 29, 2026

(54) SOLID FORMS OF MESEMBRANOL AND THERAPEUTIC USES THEREOF

(71) Applicant: Sensorium Therapeutics, Inc., Boston, MA (US)

(72) Inventors: Yunmin Yang, Suzhou (CN); Le Chang, Suzhou (CN)

(21) Appl. No.: 19/157,575

(22) PCT Filed: Feb. 16, 2024
(86) PCT No.: PCT/US24/16163

§ 371 Date: Aug. 18, 2025

(30) Foreign Application Priority Data
Feb. 17, 2023 (WO) PCT/CN2023/076677

(51) Int. Cl. C07D 209/12

(52) U.S. Cl. CPC C07D 209/12

(57) ABSTRACT

Solid forms of mesembranol are provided, along with related methods of manufacture. The solid form compositions are useful, for example, in the preparation of pharmaceutical compositions.

### RELATED APPLICATIONS

This patent application claims the benefit of priority to Patent Cooperation Treaty (PCT) Patent Application Number PCT/CN2023/076677, filed Feb. 17, 2023; the entire content of which is hereby incorporated by reference in its entirety.

### TECHNICAL FIELD

The present disclosure relates to the field of medicine, including the discovery of alkaloid compounds useful for inhibiting the serotonin transporter protein (5-HTT).

### BACKGROUND

Plants of the genus Sceletium contain indole alkaloids having biological activity useful in treating mental health conditions such as mild to moderate depression. Natural extracts of Sceletium tortuosum, an indigenous herb of South Africa also referred to as “kougoed”, “channa” or “kanna,” can contain the pharmacologically active alkaloids. Mesembrine, mesembrenone and mesembrenol, among the alkaloids shown below, are present in Sceletium tortuosum extracts used for treatment of anxiety, stress and mental health conditions.

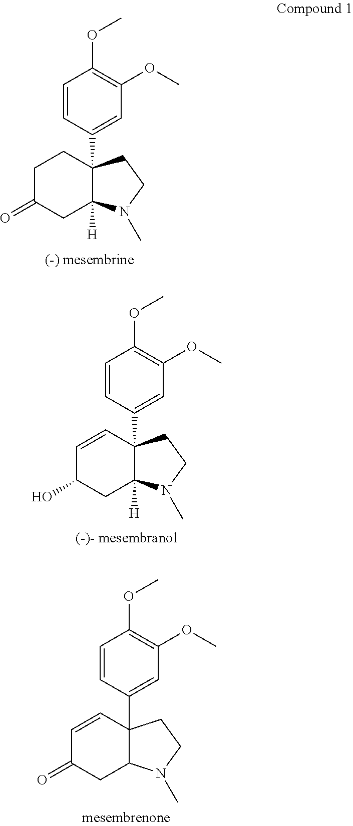 [SMILES: COc1ccc([C@@]23CCC(=O)C[C@@H]2N(C)CC3)cc1OC ; COc1ccc([C@@]23C=C[C@@H](O)C[C@@H]2N(C)CC3)cc1OC ; COc1ccc(C23C=CC(=O)CC2N(C)CC3)cc1OC]

Mesembranol is not well-characterized pharmacologically and concentration of this alkaloid in plant extracts are comparatively low relative to the other biologically active Sceletium tortuosum alkaloids. In Sceletium tortuosum extract mesembranol has low concentrations compared to the other major alkaloids. Therefore, mesembranol has not been well characterized in the scientific literature. Its therapeutic use has been limited by the low abundance, variability and instability of these alkaloids in natural extract products, and the instability and pharmacokinetic profile of these compounds as obtained from natural products. Naturally occurring mesembranol is a serotonin transporter inhibitor with high specificity relative to the other pharmacological targets. Sceletium tortuosum alkaloid extract does not yield sufficient levels of mesembranol relative to other components, and the extract could exert other additional pharmacological effects that are undesirable for a therapeutic regimen for anxiety or depression.

Polymorphs, solvates and salts of various drugs have been described in the literature as imparting novel properties to the drugs. Organic small drug molecules have a tendency to self-assemble into various polymorphic forms depending on the environment that drives the self-assembly. Heat and solvent mediated effects can also lead to changes that transform one polymorphic form into another.

Identifying which polymorphic form is the most stable under each condition of interest and the processes that lead to changes in the polymorphic form is crucial to the design of the drug manufacturing process in order to ensure that the final product is in its preferred polymorphic form. Different polymorphic forms of an active pharmaceutical ingredient (API) can lead to changes in the drug's solubility, dissolution rate, pharmacokinetics, and ultimately its bioavailability and efficacy in patients.

### SUMMARY

Described are solid forms of mesembranol (e.g., (−) mesembranol (Compound 1)). In some embodiments, solid forms of mesembranol comprise the product of the processes disclosed herein.

In some embodiments, a pharmaceutical composition comprises a solid form described herein; and a pharmaceutically acceptable excipient.

In some embodiments, a pharmaceutical composition is formed by a process comprising dissolving a solid form described herein.

In some embodiments, a method of treating a mental health disorder comprises administering to a mammal in need thereof an effective amount of a solid form described herein or a pharmaceutical composition described herein. In some embodiments, the mental health disorder is anxiety, stress, or depression. In some embodiments, the mammal is a human.

In some embodiments, a composition provided herein comprises a salt of mesembranol selected from the group consisting of a hydrochloride salt of mesembranol, a sulfate salt of mesembranol, a malate salt of mesembranol, a maleate salt of mesembranol, a gentisate salt of mesembranol, a phosphate salt of mesembranol, a tartrate salt of mesembranol, a fumarate salt of mesembranol, a naphthalene-1,5-disulfonate salt of mesembranol, an oxalate salt of mesembranol and a hydrobromide salt of mesembranol.

In some embodiments, the composition comprises a salt of (−) mesembranol. In some embodiments, the composition comprises the sulfate salt of (−) mesembranol. In some embodiments, the sulfate salt of (−) mesembranol is solid Form A of the sulfate salt of (−) mesembranol. In some embodiments, the sulfate salt of (−) mesembranol is characterized by an X-ray powder diffraction (XRPD) pattern comprising 2-theta peaks (2-theta±0.2) at 16.1°, 16.5°, 17.5°, 18.1°, and 18.7°. In some embodiments, the sulfate salt of (−) mesembranol is characterized by a sample weight loss of up to about 3.9% upon heating to 200° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, the sulfate salt of (−) mesembranol is characterized by a DSC having an endotherm at about 157° C. to about 163° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the composition comprises the malate salt of (−) mesembranol. In some embodiments, the malate salt of (−) mesembranol is solid Form A of the malate salt of (−) mesembranol. In some embodiments, the malate salt of (−) mesembranol is characterized by an X-ray powder diffraction (XRPD) pattern comprising 2-theta peaks (2-theta±0.2) at 13.1°, 16.0°, 20.8°, 21.5°, and 23.7°. In some embodiments, the malate salt of (−) mesembranol is characterized by a sample weight loss of up to about 1.9% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, the malate salt of (−) mesembranol is characterized by a DSC having an endotherm at about 140° C. to about 145° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the malate salt of (−) mesembranol is solid Form B of the malate salt of (−) mesembranol. In some embodiments, the malate salt of (−) mesembranol is characterized by an X-ray powder diffraction (XRPD) pattern comprising 2-theta peaks (2-theta±0.2) at 4.6°, 9.0°, 12.5°, 12.8°, 13.5°, 16.4°, 16.9°, 18.1°, 19.0°, 19.7°, 19.9°, 20.2°, 21.6°, 24.7°, 26.5°, 27.7°, and 30.2°. In some embodiments, the malate salt of (−) mesembranol is characterized by a sample weight loss of up to about 4.4% upon heating to 110° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, the malate salt of (−) mesembranol is characterized by a DSC having an endotherm at about 99° C. to about 104° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the composition comprises the gentisate salt of (−) mesembranol. In some embodiments, the gentisate salt of (−) mesembranol is solid Form A of the gentisate salt of (−) mesembranol. In some embodiments, the gentisate salt of (−) mesembranol is characterized by an X-ray powder diffraction (XRPD) pattern comprising 2-theta peaks (2-theta±0.2) at 9.0°, 16.6°, 17.1°, 19.0°, and 24.2°. In some embodiments, the gentisate salt of (−) mesembranol is characterized by a sample weight loss of up to about 2.1% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, the gentisate salt of (−) mesembranol is characterized by a DSC having an endotherm at about 192° C. to about 197° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the composition comprises the maleate salt of (−) mesembranol. In some embodiments, the maleate salt of (−) mesembranol is solid Form A of the maleate salt of (−) mesembranol. In some embodiments, the maleate salt of (−) mesembranol is characterized by an X-ray powder diffraction (XRPD) pattern comprising 2-theta peaks (2-theta±0.2) at 14.2°, 15.6°, 16.4°, 19.9°, and 22.1°. In some embodiments, the maleate salt of (−) mesembranol is characterized by a sample weight loss of up to about 1.3% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, the maleate salt of (−) mesembranol is characterized by a DSC having an endotherm at about 165° C. to about 170° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the composition comprises a hydrochloride salt of (−) mesembranol. In some embodiments, the hydrochloride salt of (−) mesembranol is solid Form A of the hydrochloride salt of (−) mesembranol. In some embodiments, the hydrochloride salt of (−) mesembranol is characterized by an X-ray powder diffraction (XRPD) pattern comprising 2-theta peaks (2-theta±0.2) at 16.6°, 17.0°, 18.5°, 18.8°, and 26.7°. In some embodiments, the hydrochloride salt of (−) mesembranol is characterized by a sample weight loss of up to about 8.5% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, the hydrochloride salt of (−) mesembranol is characterized by a DSC having an endotherm at about 197° C. to about 202° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the composition comprises the hydrochloride salt of (−) mesembranol as solid Form B of the hydrochloride salt of (−) mesembranol. In some embodiments, the hydrochloride salt of (−) mesembranol is characterized by an X-ray powder diffraction (XRPD) pattern comprising 2-theta peaks (2-theta±0.2) at 13.4°, 16.8°, 18.2°, 19.6°, and 19.8°. In some embodiments, the hydrochloride salt of (−) mesembranol is characterized by a sample weight loss of up to about 12% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, the hydrochloride salt of (−) mesembranol is characterized by a DSC having an endotherm at about 195° C. to about 200° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the composition comprises the hydrochloride salt of (−) mesembranol as solid Form C of the hydrochloride salt of (−) mesembranol. In some embodiments, the hydrochloride salt of (−) mesembranol is characterized by an X-ray powder diffraction (XRPD) pattern comprising 2-theta peaks (2-theta±0.2) at 9.0°, 12.4°, 16.9°, 17.9°, and 26.1°. In some embodiments, the hydrochloride salt of (−) mesembranol is characterized by a sample weight loss of up to about 17% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, the hydrochloride salt of (−) mesembranol is characterized by a DSC having an endotherm at about 193° C. to about 197° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the composition comprises a phosphate salt of (−) mesembranol. In some embodiments, the phosphate salt of (−) mesembranol is solid Form A of the phosphate salt of (−) mesembranol. In some embodiments, the phosphate salt of (−) mesembranol is characterized by an X-ray powder diffraction (XRPD) pattern comprising 2-theta peaks (2-theta±0.2) at 3.9°, 15.8°, 17.5°, 19.5°, and 22.4°. In some embodiments, the phosphate salt of (−) mesembranol is characterized by a sample weight loss of up to about 10% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, the phosphate salt of (−) mesembranol is characterized by a DSC having an endotherm at about 102° C. to about 107° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the composition comprises a tartrate salt of (−) mesembranol. In some embodiments, the tartrate salt of (−) mesembranol is solid Form A of the tartrate salt of (−) mesembranol. In some embodiments, the tartrate salt of (−) mesembranol is characterized by an X-ray powder diffraction (XRPD) pattern comprising 2-theta peaks (2-theta±0.2) at 5.0°, 14.1°, 14.9°, 17.3°, and 18.4°. In some embodiments, the tartrate salt of (−) mesembranol is characterized by a sample weight loss of up to about 7% upon heating to 160° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, the tartrate salt of (−) mesembranol is characterized by a DSC having an endotherm at about 94° C. to about 99° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the composition comprises a fumarate salt of (−) mesembranol. In some embodiments, the fumarate salt of (−) mesembranol is solid Form A of the fumarate salt of (−) mesembranol. In some embodiments, the fumarate salt of (−) mesembranol is characterized by an X-ray powder diffraction (XRPD) pattern comprising 2-theta peaks (2-theta±0.2) at 12.7°, 18.4°, 18.8°, 19.7°, and 23.1°. In some embodiments, the fumarate salt of (−) mesembranol is characterized by a sample weight loss of up to about 2% upon heating to 160° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, the fumarate salt of (−) mesembranol is characterized by a DSC having an endotherm at about 178° C. to about 183° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the composition comprises a naphthalene-1,5-disulfonate salt of (−) mesembranol. In some embodiments, the naphthalene-1,5-disulfonate salt of (−) mesembranol is solid Form A of the naphthalene-1,5-disulfonate salt of (−) mesembranol. In some embodiments, the naphthalene-1,5-disulfonate salt of (−) mesembranol is characterized by an X-ray powder diffraction (XRPD) pattern comprising 2-theta peaks (2-theta±0.2) at 13.9°, 15.2°, 16.4°, 18.1°, and 19.5°. In some embodiments, the naphthalene-1,5-disulfonate salt of (−) mesembranol is characterized by a sample weight loss of up to about 11% upon heating to 180° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, the naphthalene-1,5-disulfonate salt of (−) mesembranol is characterized by a DSC having an endotherm at about 193° C. to about 198° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the composition comprises an oxalate salt of (−) mesembranol. In some embodiments, the oxalate salt of (−) mesembranol is solid Form A of the oxalate salt of (−) mesembranol. In some embodiments, the oxalate salt of (−) mesembranol is characterized by an X-ray powder diffraction (XRPD) pattern comprising 2-theta peaks (2-theta±0.2) at 12.0°, 15.9°, 18.3°, 19.4°, and 20.0°. In some embodiments, the oxalate salt of (−) mesembranol is characterized by a sample weight loss of up to about 9% upon heating to 100° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, the oxalate salt of (−) mesembranol is characterized by a DSC having an endotherm at about 95° C. to about 100° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the composition comprises a hydrobromide salt of (−) mesembranol. In some embodiments, the hydrobromide salt of (−) mesembranol is solid Form A of the hydrobromide salt of (−) mesembranol. In some embodiments, the hydrobromide salt of (−) mesembranol is characterized by an X-ray powder diffraction (XRPD) pattern comprising 2-theta peaks (2-theta±0.2) at 16.4°, 17.2°, 18.6°, 23.4°, and 26.2°. In some embodiments, the hydrobromide salt of (−) mesembranol is characterized by a sample weight loss of up to about 5% upon heating to 80° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, the hydrobromide salt of (−) mesembranol is characterized by a DSC having an endotherm at about 200° C. to about 205° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the composition comprises a solid form of (−) mesembranol selected from the group consisting of:
a.) Sulfate Form A having an X-ray powder diffraction (XRPD) pattern comprising a 2-theta peak (2-theta±0.2) at 16.1°;
b.) Malate Form A having an X-ray powder diffraction (XRPD) pattern comprising a 2-theta peak (2-theta±0.2) at 13.1°;
c.) Malate Form B having an X-ray powder diffraction (XRPD) pattern comprising a 2-theta peak (2-theta±0.2) at 20.2;
d.) Gentisate Form A having an X-ray powder diffraction (XRPD) pattern comprising a 2-theta peak (2-theta±0.2) at 16.6°;
e.) Maleate Form A having an X-ray powder diffraction (XRPD) pattern comprising a 2-theta peak (2-theta±0.2) at 14.2°;
f.) HCl Form A having an X-ray powder diffraction (XRPD) pattern comprising a 2-theta peak (2-theta±0.2) at 18.5°;
g.) HCl Form B having an X-ray powder diffraction (XRPD) pattern comprising a 2-theta peak (2-theta±0.2) at 19.8°;
h.) HCl Form C having an X-ray powder diffraction (XRPD) pattern comprising a 2-theta peak (2-theta±0.2) at 9.0°;
i.) Phosphate Form A having an X-ray powder diffraction (XRPD) pattern comprising a 2-theta peak (2-theta±0.2) at 3.9°;
j.) Tartrate Form A having an X-ray powder diffraction (XRPD) pattern comprising a 2-theta peak (2-theta±0.2) at 5.0°;
k.) Fumarate Form A having an X-ray powder diffraction (XRPD) pattern comprising a 2-theta peak (2-theta±0.2) at 12.7°;
l.) Napthalene-1,5-disulfonate Form A having an X-ray powder diffraction (XRPD) pattern comprising a 2-theta peak (2-theta±0.2) at 18.1°;
m.) Oxalate Form A having an X-ray powder diffraction (XRPD) pattern comprising a 2-theta peak (2-theta±0.2) at 15.9°; and
n.) HBr Form A having an X-ray powder diffraction (XRPD) pattern comprising a 2-theta peak (2-theta±0.2) at 16.4°.

In some embodiments, a pharmaceutical composition comprises a solid form of any one of the salt forms of (−) mesembranol in any one of the solid forms disclosed herein, and a pharmaceutically acceptable excipient.

In some embodiments, a method of treating a mental health disorder, comprises administering to a mammal in need thereof an effective amount of a solid form of mesembranol according to any one of the salt forms of (−) mesembranol disclosed herein in any one of the solid forms disclosed herein, or a pharmaceutical composition comprising the salt forms of (−) mesembranol disclosed herein in any one of the solid forms disclosed herein.

### BRIEF DESCRIPTION OF THE DRAWINGS

FIG. 1A is an X-Ray powder diffraction (XRPD) pattern obtained from a sample of solid Freeform Form A of Compound 1.

FIG. 1B is a combined TGA thermogram of material analyzed from room temperature to 350° C. at 10° C. per minute and DSC thermogram of material analyzed from room temperature to 250° C. at 10° C. per minute obtained from the Freeform Form A of Compound 1.

FIG. 2A is an X-Ray powder diffraction (XRPD) pattern obtained from sample of solid Sulfate Form A of Compound 1.

FIG. 2B is a combined TGA thermogram of material analyzed from room temperature to 350° C. at 10° C. per minute and DSC thermogram of material analyzed from room temperature to 250° C. at 10° C. per minute obtained from the Sulfate Form A of Compound 1.

FIG. 3A is an X-Ray powder diffraction (XRPD) pattern obtained from sample of solid Malate Form A of Compound 1.

FIG. 3B is a combined TGA thermogram of material analyzed from room temperature to 350° C. at 10° C. per minute and DSC thermogram of material analyzed from room temperature to 250° C. at 10° C. per minute obtained from the Malate Form A of Compound 1.

FIG. 4A is an X-Ray powder diffraction (XRPD) pattern obtained from sample of solid Malate Form B of Compound 1.

FIG. 4B is a combined TGA thermogram of material analyzed from room temperature to 350° C. at 10° C. per minute and DSC thermogram of material analyzed from room temperature to 250° C. at 10° C. per minute obtained from the Malate Form B of Compound 1.

FIG. 5A is an X-Ray powder diffraction (XRPD) pattern obtained from sample of solid Gentisate Form A of Compound 1.

FIG. 5B is a combined TGA thermogram of material analyzed from room temperature to 350° C. at 10° C. per minute and DSC thermogram of material analyzed from room temperature to 250° C. at 10° C. per minute obtained from the Gentisate Form A of Compound 1.

FIG. 6A is an X-Ray powder diffraction (XRPD) pattern obtained from sample of solid Maleate Form A of Compound 1.

FIG. 6B is a combined TGA thermogram of material analyzed from room temperature to 350° C. at 10° C. per minute and DSC thermogram of material analyzed from room temperature to 250° C. at 10° C. per minute obtained from the Maleate Form A of Compound 1.

FIG. 6C is an X-Ray powder diffraction (XRPD) pattern obtained from sample of solid Maleate Form A of Compound 1 before and after heating to 70° C. and cooling to room temperature.

FIG. 6D is a combined TGA thermogram of material analyzed from room temperature to 350° C. at 10° C. per minute and DSC thermogram of material analyzed from room temperature to 250° C. at 10° C. per minute obtained from the Maleate Form A of Compound 1 after heating to 70° C. and cooling to room temperature.

FIG. 7A is an X-Ray powder diffraction (XRPD) pattern obtained from sample of solid HCl Form A of Compound 1.

FIG. 7B is a combined TGA thermogram of material analyzed from room temperature to 350° C. at 10° C. per minute and DSC thermogram of material analyzed from room temperature to 250° C. at 10° C. per minute obtained from the HCl Form A of Compound 1.

FIG. 7C is an X-Ray powder diffraction (XRPD) pattern obtained from sample of solid HCl Form A of Compound 1 before and after heating to 100° C. and cooling to room temperature.

FIG. 7D is a combined TGA thermogram of material analyzed from room temperature to 350° C. at 10° C. per minute and DSC thermogram of material analyzed from room temperature to 250° C. at 10° C. per minute obtained from the HCl Form A of Compound 1 after heating to 100° C. and cooling to room temperature.

FIG. 8A is an X-Ray powder diffraction (XRPD) pattern obtained from sample of solid HCl Form B of Compound 1.

FIG. 8B is a combined TGA thermogram of material analyzed from room temperature to 350° C. at 10° C. per minute and DSC thermogram of material analyzed from room temperature to 250° C. at 10° C. per minute obtained from the HCl Form B of Compound 1.

FIG. 8C is an X-Ray powder diffraction (XRPD) pattern obtained from sample of solid HCl Form B of Compound 1 before and after heating to 100° C. and cooling to room temperature.

FIG. 9A is an X-Ray powder diffraction (XRPD) pattern obtained from sample of solid HCl Form C of Compound 1.

FIG. 9B is a combined TGA thermogram of material analyzed from room temperature to 350° C. at 10° C. per minute and DSC thermogram of material analyzed from room temperature to 250° C. at 10° C. per minute obtained from the HCl Form C of Compound 1.

FIG. 10A is an X-Ray powder diffraction (XRPD) pattern obtained from sample of solid Phosphate Form A of Compound 1.

FIG. 10B is a combined TGA thermogram of material analyzed from room temperature to 350° C. at 10° C. per minute and DSC thermogram of material analyzed from room temperature to 250° C. at 10° C. per minute obtained from the Phosphate Form A of Compound 1.

FIG. 11A is an X-Ray powder diffraction (XRPD) pattern obtained from sample of solid Tartrate Form A of Compound 1.

FIG. 11B is a combined TGA thermogram of material analyzed from room temperature to 350° C. at 10° C. per minute and DSC thermogram of material analyzed from room temperature to 250° C. at 10° C. per minute obtained from the Tartrate Form A of Compound 1.

FIG. 12A is an X-Ray powder diffraction (XRPD) pattern obtained from sample of solid Fumarate Form A of Compound 1.

FIG. 12B is a combined TGA thermogram of material analyzed from room temperature to 350° C. at 10° C. per minute and DSC thermogram of material analyzed from room temperature to 250° C. at 10° C. per minute obtained from the Fumarate Form A of Compound 1.

FIG. 13A is an X-Ray powder diffraction (XRPD) pattern obtained from sample of solid Naphthalene-1,5-disulfonate Form A of Compound 1.

FIG. 13B is a combined TGA thermogram of material analyzed from room temperature to 350° C. at 10° C. per minute and DSC thermogram of material analyzed from room temperature to 250° C. at 10° C. per minute obtained from the Naphthalene-1,5-disulfonate Form A of Compound 1.

FIG. 14A is an X-Ray powder diffraction (XRPD) pattern obtained from sample of solid Oxalate Form A of Compound 1.

FIG. 14B is a combined TGA thermogram of material analyzed from room temperature to 350° C. at 10° C. per minute and DSC thermogram of material analyzed from room temperature to 250° C. at 10° C. per minute obtained from the Oxalate Form A of Compound 1.

FIG. 15A is an X-Ray powder diffraction (XRPD) pattern obtained from sample of solid HBr Form A of Compound 1.

FIG. 15B is a combined TGA thermogram of material analyzed from room temperature to 350° C. at 10° C. per minute and DSC thermogram of material analyzed from room temperature to 250° C. at 10° C. per minute obtained from the HBr Form A of Compound 1.

FIG. 15C is an X-Ray powder diffraction (XRPD) pattern obtained from sample of solid HBr Form A of Compound 1 before and after heating to 100° C. and cooling to room temperature.

FIG. 15D is a combined TGA thermogram of material analyzed from room temperature to 350° C. at 10° C. per minute and DSC thermogram of material analyzed from room temperature to 250° C. at 10° C. per minute obtained from the HBr Form A of Compound 1 after heating to 100° C. and cooling to room temperature.

### DETAILED DESCRIPTION OF THE INVENTION

Applicants have discovered solid forms of mesembranol (e.g., (−) mesembranol (Compound 1)). Although (−) mesembranol is bioactive with certain desirable pharmacologic effects, certain other properties are less than ideal for use as a therapeutic. Solid forms of mesembranol (e.g., crystalline salts of mesembranol) are described herein.

Mesembranol can occur in solid forms as an amorphous solid form or in a crystalline solid form or in mixtures of solid forms. Crystalline solid forms of mesembranol can exist in one or more unique solid forms, which can additionally comprise one or more equivalents of water or solvent (i.e., hydrates or solvates, respectively).

Crystalline form(s) of mesembranol having distinct characteristic XRPD peaks are provided herein. Accordingly, provided herein are crystalline mesembranol solid forms, pharmaceutical compositions thereof, methods of preparing those crystalline mesembranol solid forms, and methods of use thereof.

Compositions comprising the free base of Compound 1 in certain solid forms are provided. Compound 1 can occur in solid forms as an amorphous solid form or in a crystalline solid form or in mixtures of solid forms. Crystalline solid forms of Compound 1 can exist in one or more unique solid forms, which can additionally comprise one or more equivalents of water or solvent (i.e., hydrates or solvates, respectively). Crystalline form(s) of Compound 1 having distinct characteristic XRPD peaks are provided herein. Accordingly, provided herein are crystalline Compound 1 solid forms, pharmaceutical compositions thereof, and methods of preparing those crystalline Compound 1 solid forms and methods of use thereof.

### Salt Forms of Compound 1

Various crystalline salts of Compound 1 were prepared, including (but not limited to) crystalline tartaric acid, fumaric acid, malic acid, maleic acid, naphthalene-1,5-disulfonic acid, hydrobromic acid, hydrochloric acid, oxalic acid, phosphoric acid, sulfuric acid, or gentisic acid. In some embodiments, a composition comprises a solid form of mesembranol (e.g., Compound 1; (−) mesembranol) in combination with one or more compounds selected from the group consisting of tartaric acid, fumaric acid, malic acid, maleic acid, naphthalene-1,5-disulfonic acid, hydrobromic acid, hydrochloric acid, oxalic acid, phosphoric acid, sulfuric acid, or gentisic acid

### Sulfate Form A

In some embodiments, solid forms of a sulfate salt of Compound 1 are provided. Sulfate Form A is a solid form of the sulfate salt of Compound 1 that can be identified by a XRPD pattern comprising certain characteristic peaks, and/or by one or more other techniques including DSC, TGA, and/or DVS.

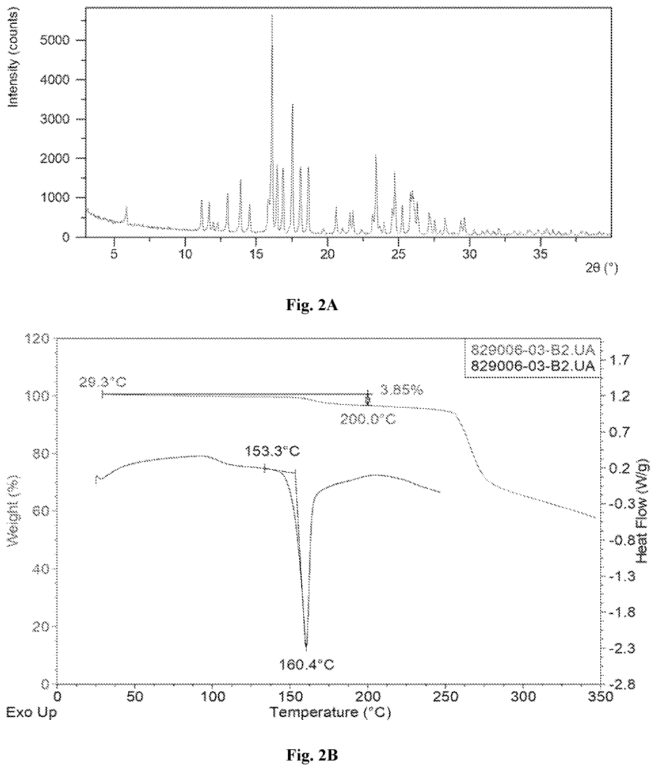

Samples of Sulfate Form A were prepared according to the Example 1 and characterized by XRPD (e.g., a pattern the same or substantially similar to or not dissimilar to FIG. 2A), TGA (e.g., a thermogram the same or substantially similar to or not dissimilar to FIG. 2B), and DSC (e.g., a thermogram the same or substantially similar to or not dissimilar to FIG. 2B).

In some embodiments, a solid sulfate salt form of mesembranol, such as Sulfate Form A, has an X-ray powder diffraction (XRPD) pattern comprising 2-theta peaks (2 theta±0.2) at 16.1°, 16.5°, 17.5°, 18.1°, and 18.7°. In some embodiments, a solid sulfate salt form of mesembranol, such as Sulfate Form A, has an X-ray powder diffraction (XRPD) pattern comprising 2-theta peaks (2 theta±0.2) at 11.2°, 11.7°, 13.0°, 13.9°, 14.5°, 15.8°, 16.1°, 16.5°, 16.9°, 17.5°, 18.1°, 18.7°, 20.6°, 21.8°, 23.4°, 24.6°, 24.7°, 25.3°, 25.8°, 26.0°, and 26.3°.

In some embodiments, a solid sulfate salt form of mesembranol, such as Sulfate Form A, has an X-ray powder diffraction (XRPD) pattern comprising 2-theta peaks according to Table 2 having a relative intensity of greater than 10%. In some embodiments, a solid sulfate salt form of mesembranol, such as Sulfate Form A, has an X-ray powder diffraction (XRPD) pattern according to FIG. 2A.

In some embodiments, a solid sulfate salt form of mesembranol, such as Sulfate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 16.1 | 5.5 |
| 16.5 | 5.4 |
| 17.5 | 5.1 |
| 18.1 | 4.9 |
| 18.7 | 4.8 |

In some embodiments, a solid sulfate salt form of mesembranol, such as Sulfate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 11.2 | 7.9 |
| 11.7 | 7.6 |
| 13.0 | 6.8 |
| 13.9 | 6.4 |
| 14.5 | 6.1 |
| 15.8 | 5.6 |
| 16.1 | 5.5 |
| 16.5 | 5.4 |
| 16.9 | 5.2 |
| 17.5 | 5.1 |
| 18.1 | 4.9 |
| 18.7 | 4.8 |
| 20.6 | 4.3 |
| 21.8 | 4.1 |
| 23.4 | 3.8 |
| 24.6 | 3.6 |
| 24.7 | 3.6 |
| 25.3 | 3.5 |
| 25.8 | 3.4 |
| 26.0 | 3.4 |
| 26.3 | 3.4 |

In some embodiments, a solid sulfate salt form of mesembranol, such as Sulfate Form A, is characterized by a TGA thermogram according to FIG. 2B. In some embodiments, a solid sulfate salt form of mesembranol, such as Sulfate Form A, is further characterized by a sample weight loss of up to about 3.9% upon heating up to 200° C., as measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, a solid sulfate salt form of mesembranol, such as Sulfate Form A, is characterized by a differential scanning calorimetry (DSC) thermogram according to FIG. 2B. In some embodiments, a solid sulfate salt form of mesembranol, such as Sulfate Form A, is further characterized by a DSC having an endotherm at about 157° C. to about 163° C. (peak temperature), as measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the stoichiometric ratio of SO₄²⁻/freeform is about 1.0, as measured by UPLC/IC. In some embodiments, the purity of Sulfate Form A is about 99.83 area %, as measured by UPLC.

### Malate Form A

In some embodiments, solid forms of a malate salt of Compound 1 are provided. Malate Form A is a solid form of the malate salt of Compound 1 that can be identified by a XRPD pattern comprising certain characteristic peaks, and/or by one or more other techniques including DSC, TGA, and/or DVS.

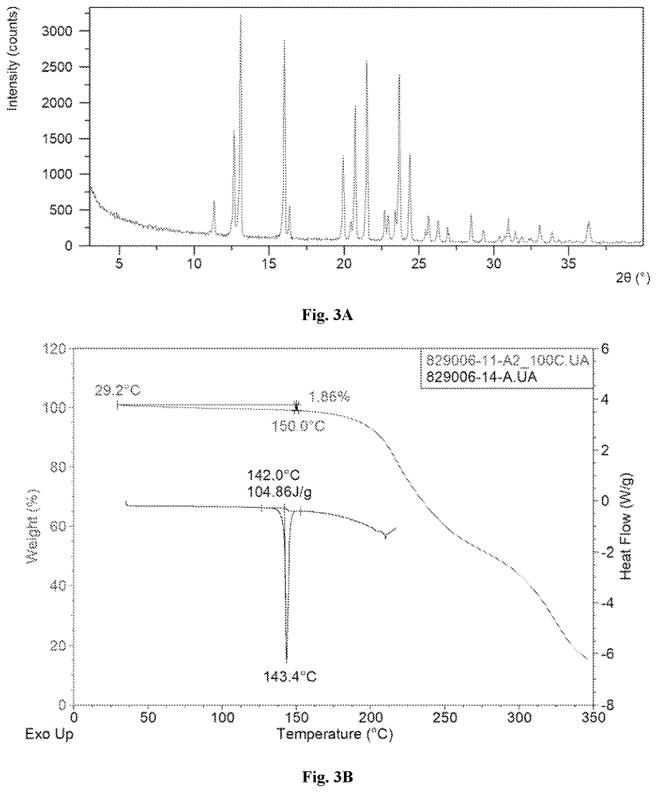

Samples of Malate Form A were prepared according to the Example 2 and characterized by XRPD (e.g., a pattern the same or substantially similar to or not dissimilar to FIG. 3A), TGA (e.g., a thermogram the same or substantially similar to or not dissimilar to FIG. 3B), and DSC (e.g., a thermogram the same or substantially similar to or not dissimilar to FIG. 3B).

In some embodiments, a solid malate salt form of mesembranol, such as Malate Form A, has an X-ray powder diffraction (XRPD) pattern comprising 2-theta peaks (2 theta±0.2) at 13.1°, 16.0°, 20.8°, 21.5°, and 23.7°. In some embodiments, a solid malate salt form of mesembranol, such as Malate Form A, has an X-ray powder diffraction (XRPD) pattern comprising 2-theta peaks (2 theta±0.2) at 11.3°, 12.7°, 13.1°, 16.0°, 16.4°, 20.0°, 20.8°, 21.5°, 22.7°, 23.0°, 23.4°, 23.7°, 24.4°, 25.6°, 28.5°, and 31.0°.

In some embodiments, a solid malate salt form of mesembranol, such as Malate Form A, has an X-ray powder diffraction (XRPD) pattern comprising 2-theta peaks according to Table 3 having a relative intensity of greater than 10%. In some embodiments, a solid malate salt form of mesembranol, such as Malate Form A, has an X-ray powder diffraction (XRPD) pattern according to FIG. 3A.

In some embodiments, a solid malate salt form of mesembranol, such as Malate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 13.1 | 6.8 |
| 16.0 | 5.5 |
| 20.8 | 4.3 |
| 21.5 | 4.1 |
| 23.7 | 3.8 |

In some embodiments, a solid malate salt form of mesembranol, such as Malate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 11.3 | 7.8 |
| 12.7 | 7.0 |
| 13.1 | 6.8 |
| 16.0 | 5.5 |
| 16.4 | 5.4 |
| 20.0 | 4.4 |
| 20.8 | 4.3 |
| 21.5 | 4.1 |
| 22.7 | 3.9 |
| 23.0 | 3.9 |
| 23.4 | 3.8 |
| 23.7 | 3.8 |
| 24.4 | 3.6 |
| 25.6 | 3.5 |
| 28.5 | 3.1 |
| 31.0 | 2.9 |

In some embodiments, a solid malate salt form of mesembranol, such as Malate Form A, is characterized by a TGA thermogram according to FIG. 3B. In some embodiments, a solid malate salt form of mesembranol, such as Malate Form A, is further characterized by a sample weight loss of up to about 1.9% upon heating up to 150° C., as measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, a solid malate salt form of mesembranol, such as Malate Form A, is characterized by a differential scanning calorimetry (DSC) thermogram according to FIG. 3B. In some embodiments, a solid malate salt form of mesembranol, such as Malate Form A, is further characterized by a DSC having an endotherm at about 140° C. to about 145° C. (peak temperature) and/or at about 88° C. to about 93° C. (peak temperature), as measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the molar ratio of acid/freeform is about 1.0, as determined by 1H NMR. In some embodiments, the residual solvent acetone/API was about 0.02 (0.3 wt %), as determined by ¹H NMR. In some embodiments, the purity is about 99.78 area %, as measured by UPLC.

In some embodiments, after heating Malate Form A to 100° C., cooling to RT and exposure to ambient conditions, XRPD results show that several weak peaks disappear. In some embodiments, the disappeared peaks might be attributed to Malate Form B. In some embodiments, the TGA/DSC results of heated and cooled Malate Form A show a weight loss of about 1.9% up to 150° C. and one endotherm at about 143.4° C. (peak temperature).

### Malate Form B

In some embodiments, solid forms of a malate salt of Compound 1 are provided. Malate Form B is a solid form of the malate salt of Compound 1 that can be identified by a XRPD pattern comprising certain characteristic peaks, and/or by one or more other techniques including DSC, TGA, and/or DVS.

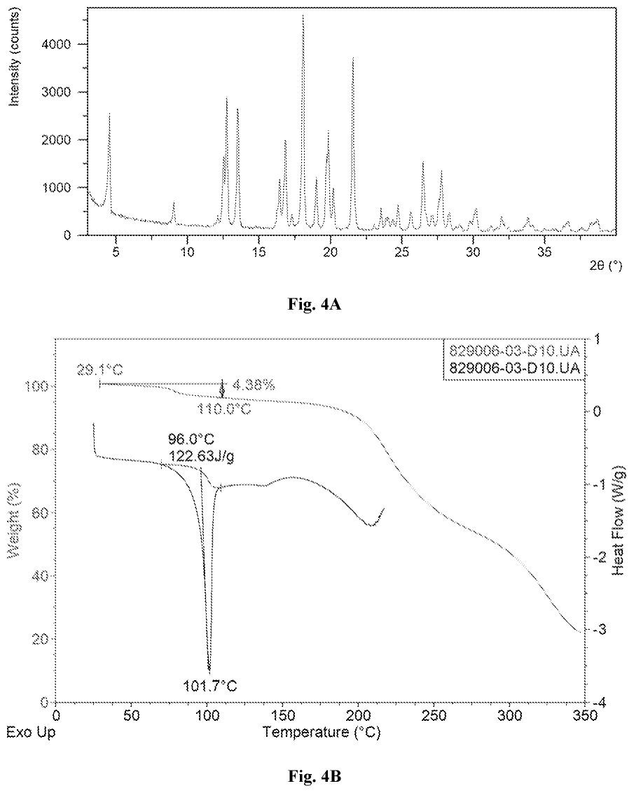

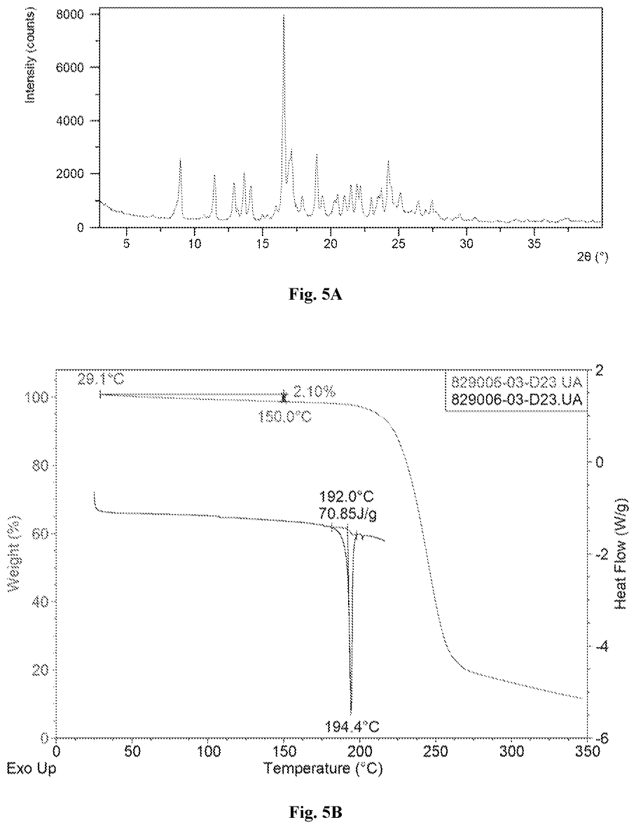

Samples of Malate Form B were prepared according to the Example 3 and characterized by XRPD (e.g., a pattern the same or substantially similar to or not dissimilar to FIG. 4A), TGA (e.g., a thermogram the same or substantially similar to or not dissimilar to FIG. 4B), and DSC (e.g., a thermogram the same or substantially similar to or not dissimilar to FIG. 4B).

In some embodiments, a solid malate salt form of mesembranol, such as Malate Form B, has an X-ray powder diffraction (XRPD) pattern comprising 2-theta peaks (2 theta±0.2) at 4.6°, 12.8°, 13.5°, 18.1°, and 21.6°. In some embodiments, a solid malate salt form of mesembranol, such as Malate Form B, has an X-ray powder diffraction (XRPD) pattern comprising 2-theta peaks (2 theta±0.2) at 4.6°, 9.0°, 12.5°, 12.8°, 13.5°, 16.4°, 16.9°, 18.1°, 19.0°, 19.7°, 19.9°, 20.2°, 21.6°, 24.7°, 26.5°, 27.7°, and 30.2°.

In some embodiments, a solid malate salt form of mesembranol, such as Malate Form B, has an X-ray powder diffraction (XRPD) pattern comprising 2-theta peaks according to Table 4 having a relative intensity of greater than 10%. In some embodiments, a solid malate salt form of mesembranol, such as Malate Form B, has an X-ray powder diffraction (XRPD) pattern according to FIG. 4A.

In some embodiments, a solid malate salt form of mesembranol, such as Malate Form B, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 4.6 | 19.4 |
| 12.8 | 6.9 |
| 13.5 | 6.5 |
| 18.1 | 4.9 |
| 21.6 | 4.1 |

In some embodiments, a solid malate salt form of mesembranol, such as Malate Form B, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 4.6 | 19.4 |
| 9.0 | 9.8 |
| 12.5 | 7.1 |
| 12.8 | 6.9 |
| 13.5 | 6.5 |
| 16.4 | 5.4 |
| 16.9 | 5.3 |
| 18.1 | 4.9 |
| 19.0 | 4.7 |
| 19.7 | 4.5 |
| 19.9 | 4.5 |
| 20.2 | 4.4 |
| 21.6 | 4.1 |
| 24.7 | 3.6 |
| 26.5 | 3.4 |
| 27.7 | 3.2 |
| 30.2 | 3.0 |

In some embodiments, a solid malate salt form of mesembranol, such as Malate Form B, is characterized by a TGA thermogram according to FIG. 4B. In some embodiments, a solid malate salt form of mesembranol, such as Malate Form B, is further characterized by a sample weight loss of up to about 4.4% upon heating to 110° C., as measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, a solid malate salt form of mesembranol, such as Malate Form B, is characterized by a differential scanning calorimetry (DSC) thermogram according to FIG. 4B. In some embodiments, a solid malate salt form of mesembranol, such as Malate Form B, is further characterized by a DSC having an endotherm at about 99° C. to about 104° C. (peak temperature), as measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the molar ratio of acid/freeform is about 1.1, as determined by 1H NMR. In some embodiments, the residual solvent 2-MeHTF/API is about 0.03 (0.5 wt %), as determined by ¹H NMR. In some embodiments, the purity of Malate Form B is about 98.9 area %, as measured by UPLC

### Gentisate Form A

In some embodiments, solid forms of a gentisate salt of Compound 1 are provided. Gentisate Form A is a solid form of the gentisate salt of Compound 1 that can be identified by a XRPD pattern comprising certain characteristic peaks, and/or by one or more other techniques including DSC, TGA, and/or DVS.

Samples of Gentisate Form A were prepared according to the Example 4 and characterized by XRPD (e.g., a pattern the same or substantially similar to or not dissimilar to FIG. 5A), TGA (e.g., a thermogram the same or substantially similar to or not dissimilar to FIG. 5B), and DSC (e.g., a thermogram the same or substantially similar to or not dissimilar to FIG. 5B).

In some embodiments, a solid gentisate salt form of mesembranol, such as Gentisate Form A, has an X-ray powder diffraction (XRPD) pattern comprising 2-theta peaks (2 theta±0.2) at 9.0°, 16.6°, 17.1°, 19.0°, and 24.2°. In some embodiments, a solid gentisate salt form of mesembranol, such as Gentisate Form A, has an X-ray powder diffraction (XRPD) pattern comprising 2-theta peaks (2 theta±0.2) at 9.0°, 11.5°, 12.9°, 13.6°, 14.2°, 16.6°, 17.1°, 17.9°, 19.0°, 19.4°, 20.5°, 21.0°, 21.5°, 21.9°, 22.2°, 23.7°, 24.2°, and 25.2°.

In some embodiments, a solid gentisate salt form of mesembranol, such as Gentisate Form A, has an X-ray powder diffraction (XRPD) pattern comprising 2-theta peaks according to Table 5 having a relative intensity of greater than 10%. In some embodiments, a solid gentisate salt form of mesembranol, such as Gentisate Form A, has an X-ray powder diffraction (XRPD) pattern according to FIG. 5A.

In some embodiments, a solid gentisate salt form of mesembranol, such as Gentisate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 9.0 | 9.9 |
| 16.6 | 5.4 |
| 17.1 | 5.2 |
| 19.0 | 4.7 |
| 24.2 | 3.7 |

In some embodiments, a solid gentisate salt form of mesembranol, such as Gentisate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 9.0 | 9.9 |
| 11.5 | 7.7 |
| 12.9 | 6.9 |
| 13.6 | 6.5 |
| 14.2 | 6.3 |
| 16.6 | 5.4 |
| 17.1 | 5.2 |
| 17.9 | 5.0 |
| 19.0 | 4.7 |
| 19.4 | 4.6 |
| 20.5 | 4.3 |
| 21.0 | 4.2 |
| 21.5 | 4.1 |
| 21.9 | 4.0 |
| 22.2 | 4.0 |
| 23.7 | 3.8 |
| 24.2 | 3.7 |
| 25.2 | 3.5 |

In some embodiments, a solid gentisate salt form of mesembranol, such as Gentisate Form A, is characterized by a TGA thermogram according to FIG. 5B. In some embodiments, a solid gentisate salt form of mesembranol, such as Gentisate Form A, is further characterized by a sample weight loss of up to about 2.1% upon heating up to 150° C., as measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, a solid gentisate salt form of mesembranol, such as Gentisate Form A, is characterized by a differential scanning calorimetry (DSC) thermogram according to FIG. 5B. In some embodiments, a solid gentisate salt form of mesembranol, such as Gentisate Form A, is further characterized by a DSC having an endotherm at about 192° C. to about 197° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the molar ratio of acid/freeform is about 1, as determined by 1H NMR. In some embodiments, the residual solvent 2-MeTHF/API is about 0.04 (0.8 wt %), as determined by ¹H NMR. In some embodiments, the purity of Gentisate Form A about 99.1 area %, as measured by UPLC.

### Maleate Form A

In some embodiments, solid forms of a maleate salt of Compound 1 are provided. Maleate Form A is a solid form of the maleate salt of Compound 1 that can be identified by a XRPD pattern comprising certain characteristic peaks, and/or by one or more other techniques including DSC, TGA, and/or DVS.

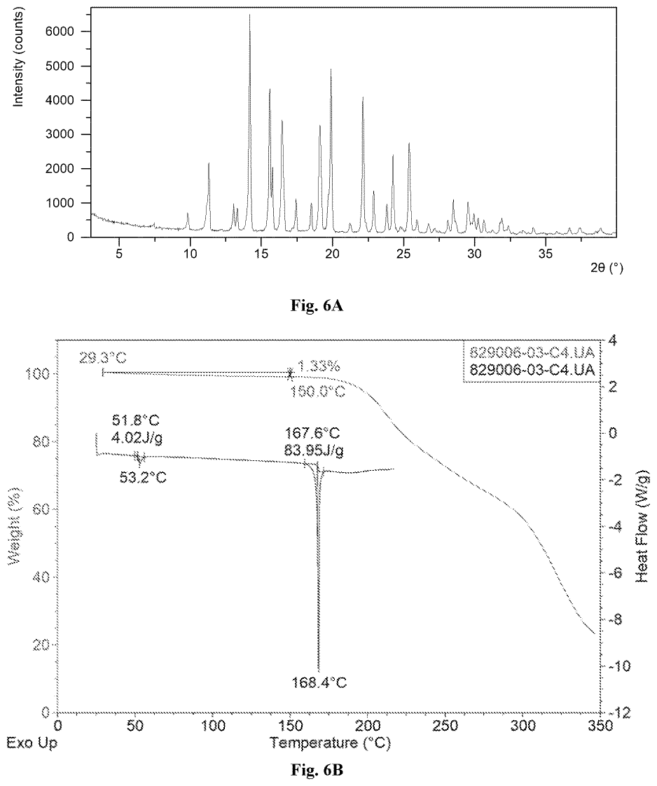

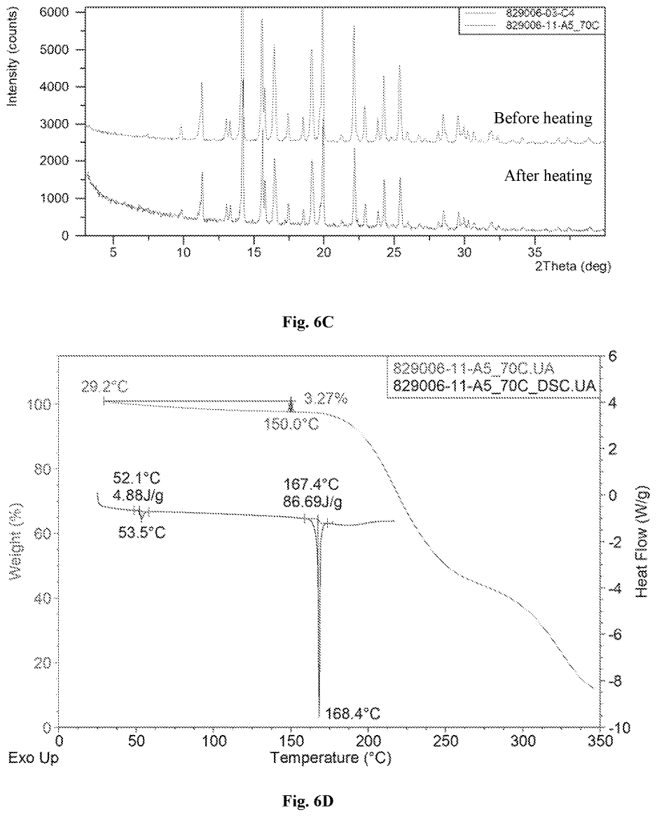

Samples of Maleate Form A were prepared according to the Example 5 and characterized by XRPD (e.g., a pattern the same or substantially similar to or not dissimilar to FIG. 6A), TGA (e.g., a thermogram the same or substantially similar to or not dissimilar to FIG. 6B), and DSC (e.g., a thermogram the same or substantially similar to or not dissimilar to FIG. 6B).

In some embodiments, a solid maleate salt form of mesembranol, such as Maleate Form A, has an X-ray powder diffraction (XRPD) pattern comprising 2-theta peaks (2 theta±0.2) at 14.2°, 15.6°, 16.4°, 19.9°, and 22.1°. In some embodiments, a solid maleate salt form of mesembranol, such as Maleate Form A, has an X-ray powder diffraction (XRPD) pattern comprising 2-theta peaks (2 theta±0.2) at 11.3°, 13.0°, 13.3°, 14.2°, 15.6°, 15.8°, 16.4°, 17.4°, 18.5°, 19.1°, 19.9°, 22.1°, 22.9°, 23.8°, 24.2°, 25.4°, 28.5°, and 29.5°.

In some embodiments, a solid maleate salt form of mesembranol, such as Maleate Form A, has an X-ray powder diffraction (XRPD) pattern comprising 2-theta peaks according to Table 6 having a relative intensity of greater than 10%. In some embodiments, a solid maleate salt form of mesembranol, such as Maleate Form A, has an X-ray powder diffraction (XRPD) pattern according to FIG. 6A.

In some embodiments, a solid maleate salt form of mesembranol, such as Maleate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 14.2 | 6.2 |
| 15.6 | 5.7 |
| 16.4 | 5.4 |
| 19.9 | 4.5 |
| 22.1 | 4.0 |

In some embodiments, a solid maleate salt form of mesembranol, such as Maleate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 11.3 | 7.8 |
| 13.0 | 6.8 |
| 13.3 | 6.7 |
| 14.2 | 6.2 |
| 15.6 | 5.7 |
| 15.8 | 5.6 |
| 16.4 | 5.4 |
| 17.4 | 5.1 |
| 18.5 | 4.8 |
| 19.1 | 4.6 |
| 19.9 | 4.5 |
| 22.1 | 4.0 |
| 22.9 | 3.9 |
| 23.8 | 3.7 |
| 24.2 | 3.7 |
| 25.4 | 3.5 |
| 28.5 | 3.1 |
| 29.5 | 3.0 |

In some embodiments, a solid maleate salt form of mesembranol, such as Maleate Form A, is characterized by a TGA thermogram according to FIG. 6B. In some embodiments, a solid maleate salt form of mesembranol, such as Maleate Form A, is further characterized by a sample weight loss of up to about 1.3% upon heating up to 150° C., as measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, a solid maleate salt form of mesembranol, such as Maleate Form A, is characterized by a differential scanning calorimetry (DSC) thermogram according to FIG. 6B. In some embodiments, a solid maleate salt form of mesembranol, such as Maleate Form A, is further characterized by a DSC having an endotherm at about 165° C. to about 170° C. (peak temperature) and/or at about 51° C. to about 56° C. (peak temperature), as measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, the molar ratio of maleic acid/freeform is about 0.9, as determined by ¹H NMR. In some embodiments, negligible solvent EtOAc is detectable, as determined by 1H NMR. In some embodiments, the purity of Maleate Form A is about 99.4 area %, as measured by UPLC.

In some embodiments, a heating experiment was performed to study the thermal signal around 53° C. In some embodiments, the XRPD results show that no form change was observed after heating Maleate Form A to 70° C., cooling to RT and exposure to ambient conditions. In some embodiments, TGA/DSC results of the heated sample demonstrate a weight loss of about 3.3% up to 150° C. and two endotherms at about 53.5° C. and about 168.4° C. (peak temperature).

### HCl Form A

In some embodiments, solid forms of an HCl salt of Compound 1 are provided. HCl Form A is a solid form of the HCl salt of Compound 1 that can be identified by a XRPD pattern comprising certain characteristic peaks, and/or by one or more other techniques including DSC, TGA, and/or DVS.

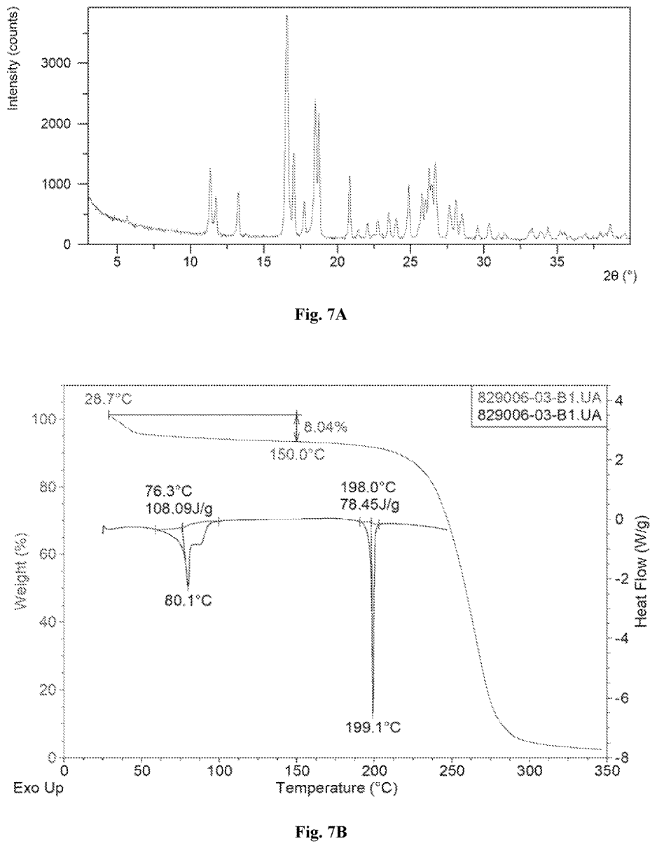

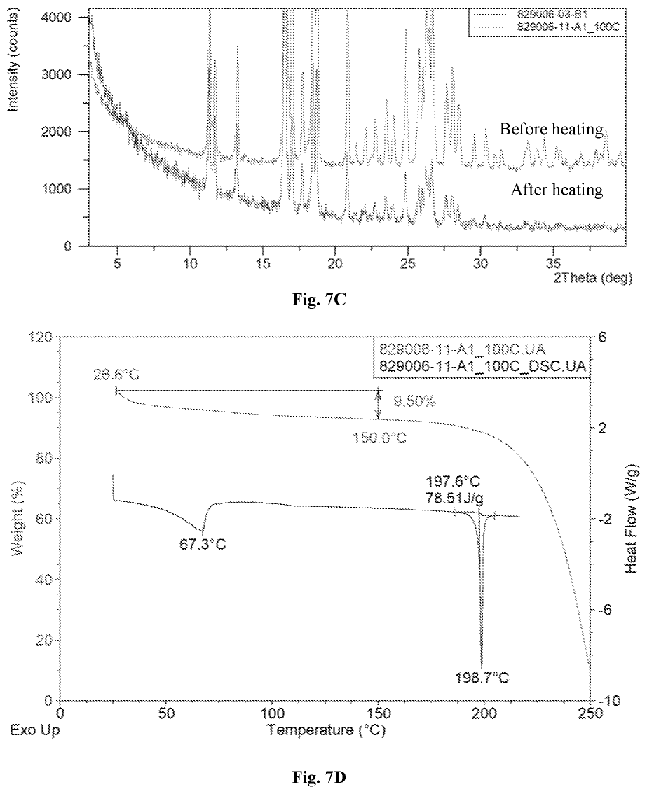

Samples of HCl Form A were prepared according to the Example 6 and characterized by XRPD (e.g., a pattern the same or substantially similar to or not dissimilar to FIG. 7A), TGA (e.g., a thermogram the same or substantially similar to or not dissimilar to FIG. 7B), and DSC (e.g., a thermogram the same or substantially similar to or not dissimilar to FIG. 7B).

In some embodiments, a solid HCl salt form of mesembranol, such as HCl Form A, has an X-ray powder diffraction (XRPD) pattern comprising 2-theta peaks (2 theta±0.2) at 16.6°, 17.0°, 18.5°, 18.8°, and 26.7°. In some embodiments, a solid HCl salt form of mesembranol, such as HCl Form A, has an X-ray powder diffraction (XRPD) pattern comprising 2-theta peaks (2 theta±0.2) at 11.4°, 11.7°, 13.2°, 16.6°, 17.0°, 17.7°, 18.5°, 18.8°, 20.9°, 23.5°, 24.9°, 25.8°, 26.0°, 26.2°, 26.7°, 27.6°, 28.1°, and 28.5°.

In some embodiments, a solid HCl salt form of mesembranol, such as HCl Form A, has an X-ray powder diffraction (XRPD) pattern comprising 2-theta peaks according to Table 7 having a relative intensity of greater than 10%. In some embodiments, a solid HCl salt form of mesembranol, such as HCl Form A, has an X-ray powder diffraction (XRPD) pattern according to FIG. 7A.

In some embodiments, a solid HCl salt form of mesembranol, such as HCl Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 16.6 | 5.3 |
| 17.0 | 5.2 |
| 18.5 | 4.8 |
| 18.8 | 4.7 |
| 26.7 | 3.3 |

In some embodiments, a solid HCl salt form of mesembranol, such as HCl Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 11.4 | 7.8 |
| 11.7 | 7.6 |
| 13.2 | 6.7 |
| 16.6 | 5.3 |
| 17.0 | 5.2 |
| 17.7 | 5.0 |
| 18.5 | 4.8 |
| 18.8 | 4.7 |
| 20.9 | 4.3 |
| 23.5 | 3.8 |
| 24.9 | 3.6 |
| 25.8 | 3.5 |
| 26.0 | 3.4 |
| 26.2 | 3.4 |
| 26.7 | 3.3 |
| 27.6 | 3.2 |
| 28.1 | 3.2 |
| 28.5 | 3.1 |

In some embodiments, a solid HCl salt form of mesembranol, such as HCl Form A, is characterized by a TGA thermogram according to FIG. 7B. In some embodiments, a solid HCl salt form of mesembranol, such as HCl Form A, is further characterized by a sample weight loss of up to about 8.5% upon heating up to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

In some embodiments, a solid HCl salt form of mesembranol, such as HCl Form A, is characterized by a differential scanning calorimetry (DSC) thermogram according to FIG. 7B. In some embodiments, a solid HCl salt form of mesembranol, such as HCl Form A, is further characterized by a DSC having an endotherm at about 197° C. to about 202° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

In some embodiments, no obvious residual solvent acetone is present in HCl Form A. In some embodiments, the stoichiometric ratio of Cl⁻/freeform is about 1.0, as determined by 1H NMR. In some embodiments, the purity of HCl Form A is about 99.8 area %, as measured by UPLC.

In some embodiments, HCl Form A shows no form change after heating to 100° C., cooling to RT and exposure to ambient conditions. In some embodiments, TGA/DSC results of heated HCl Form A demonstrates a weight loss of about 9.5% up to 150° C. and two endotherms at about 67.3° C. and about 198.7° C. (peak temperature). In some embodiments, HCl Form A is a hydrate.

### HCl Form B

In some embodiments, solid forms of an HCl salt of Compound 1 are provided. HCl Form B is a solid form of the HCl salt of Compound 1 that can be identified by a XRPD pattern comprising certain characteristic peaks, and/or by one or more other techniques including DSC, TGA, and/or DVS.

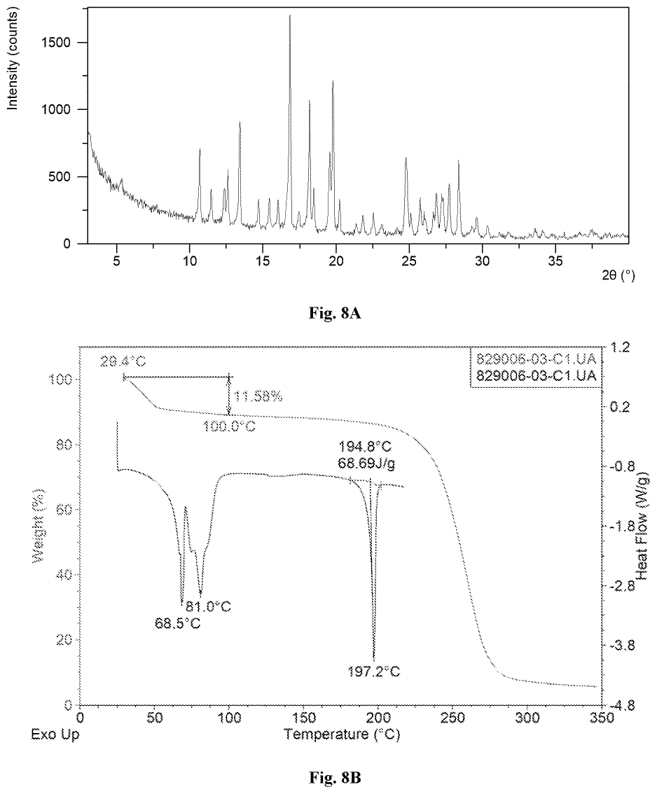

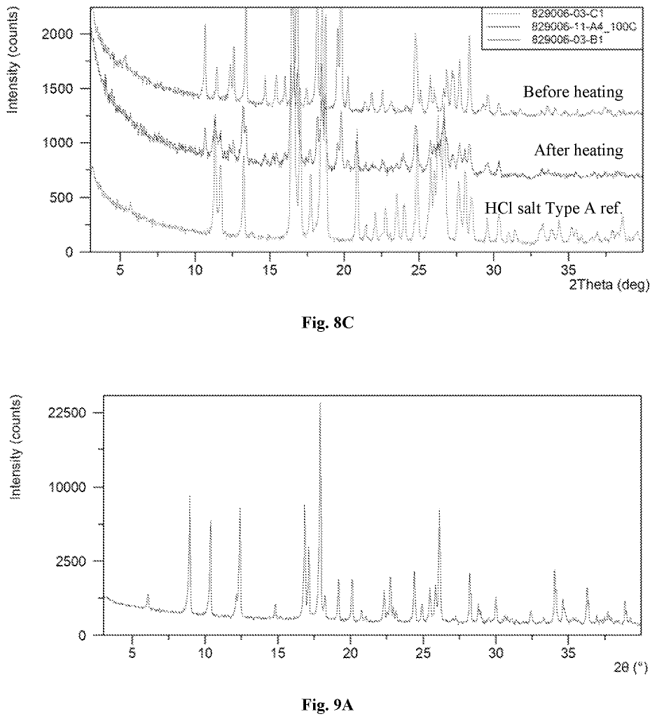

Samples of HCl Form B were prepared according to the Example 7 and characterized by XRPD (e.g., a Pattern the same or substantially similar to or not dissimilar to FIG. 8A), TGA (e.g., a thermogram the same or substantially similar to or not dissimilar to FIG. 8B), and DSC (e.g., a thermogram the same or substantially similar to or not dissimilar to FIG. 8B).

In some embodiments, a solid HCl salt form of mesembranol, such as HCl Form B, has an X-ray powder diffraction (XRPD) pattern comprising 2-theta peaks (2 theta±0.2) at 13.4°, 16.8°, 18.2°, 19.6°, and 19.8°. In some embodiments, a solid HCl salt form of mesembranol, such as HCl Form B, has an X-ray powder diffraction (XRPD) pattern comprising 2-theta peaks (2 theta±0.2) at 10.7°, 11.5°, 12.4°, 12.6°, 13.4°, 14.7°, 15.4°, 16.0°, 16.8°, 18.2°, 18.5°, 19.6°, 19.8°, 20.2°, 24.7°, 25.8°, 26.8°, 27.2°, 27.7°, and 28.4°.

In some embodiments, a solid HCl salt form of mesembranol, such as HCl Form B, has an X-ray powder diffraction (XRPD) pattern comprising 2-theta peaks according to Table 8 having a relative intensity of greater than 10%. In some embodiments, a solid HCl salt form of mesembranol, such as HCl Form B, has an X-ray powder diffraction (XRPD) pattern according to FIG. 8A.

In some embodiments, a solid HCl salt form of mesembranol, such as HCl Form B, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 13.4 | 6.6 |
| 16.8 | 5.3 |
| 18.2 | 4.9 |
| 19.6 | 4.5 |
| 19.8 | 4.5 |

In some embodiments, a solid HCl salt form of mesembranol, such as HCl Form B, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 10.7 | 8.3 |
| 11.5 | 7.7 |
| 12.4 | 7.2 |
| 12.6 | 7.0 |
| 13.4 | 6.6 |
| 14.7 | 6.0 |
| 15.4 | 5.7 |
| 16.0 | 5.5 |
| 16.8 | 5.3 |
| 18.2 | 4.9 |
| 18.5 | 4.8 |
| 19.6 | 4.5 |
| 19.8 | 4.5 |
| 20.2 | 4.4 |
| 24.7 | 3.6 |
| 25.8 | 3.5 |
| 26.8 | 3.3 |
| 27.2 | 3.3 |
| 27.7 | 3.2 |
| 28.4 | 3.2. |

In some embodiments, a solid HCl salt form of mesembranol, such as HCl Form B, is characterized by a TGA thermogram according to FIG. 8B. In some embodiments, a solid HCl salt form of mesembranol, such as HCl Form B, is further characterized by a sample weight loss of up to about 12% upon heating up to 150° C., as measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

In some embodiments, a solid HCl salt form of mesembranol, such as HCl Form B, is characterized by a differential scanning calorimetry (DSC) thermogram according to FIG. 8B. In some embodiments, a solid HCl salt form of mesembranol, such as HCl Form B, is further characterized by a DSC having an endotherm at about 195° C. to about 200° C. and/or at about 65° C. to about 70° C. and/or at about 79° C. to about 84° C. (peak temperature), as measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

In some embodiments, no obvious residual solvent EtOAc is detectable by ¹H NMR. In some embodiments, the stoichiometric ratio of Cl⁻/freeform is about 1.0 and the purity is about 99.1 area %. In some embodiments, heating HCl Form B to 100° C., cooling to RT and exposing to ambient conditions yields a mixture of HCl salt Forms A+B.

### HCl Form C

In some embodiments, solid forms of an HCl salt of Compound 1 are provided. HCl Form C is a solid form of the HCl salt of Compound 1 that can be identified by a XRPD pattern comprising certain characteristic peaks, and/or by one or more other techniques including DSC, TGA, and/or DVS.

Samples of HCl Form C were prepared according to the Example 8 and characterized by XRPD (e.g., a pattern the same or substantially similar to or not dissimilar to FIG. 9A), TGA (e.g., a thermogram the same or substantially similar to or not dissimilar to FIG. 9B), and DSC (e.g., a thermogram the same or substantially similar to or not dissimilar to FIG. 9B).

In some embodiments, a solid HCl salt form of mesembranol, such as HCl Form C, has an X-ray powder diffraction (XRPD) pattern comprising 2-theta peaks (2 theta±0.2) at 9.0°, 12.4°, 16.9°, 17.9°, and 26.1°. In some embodiments, a solid HCl salt form of mesembranol, such as HCl Form C, has an X-ray powder diffraction (XRPD) pattern comprising 2-theta peaks (2 theta±0.2) at 9.0°, 10.4°, 12.4°, 16.9°, 17.1°, 17.9°, and 26.1°.

In some embodiments, a solid HCl salt form of mesembranol, such as HCl Form C, has an X-ray powder diffraction (XRPD) pattern comprising 2-theta peaks according to Table 9 having a relative intensity of greater than 10%. In some embodiments, a solid HCl salt form of mesembranol, such as HCl Form C, has an X-ray powder diffraction (XRPD) pattern according to FIG. 9A.

In some embodiments, a solid HCl salt form of mesembranol, such as HCl Form C, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 13.4 | 6.6 |
| 16.8 | 5.3 |
| 18.2 | 4.9 |
| 19.6 | 4.5 |
| 19.8 | 4.5. |

In some embodiments, a solid HCl salt form of mesembranol, such as HCl Form C, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 9.0 | 9.9 |
| 10.4 | 8.5 |
| 12.4 | 7.1 |
| 16.9 | 5.3 |
| 17.1 | 5.2 |
| 17.9 | 5.0 |
| 26.1 | 3.4. |

In some embodiments, a solid HCl salt form of mesembranol, such as HCl Form C, is characterized by a TGA thermogram according to FIG. 9B. In some embodiments, a solid HCl salt form of mesembranol, such as HCl Form C, is further characterized by a sample weight loss of up to about 17% upon heating up to 150° C., as measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

In some embodiments, a solid HCl salt form of mesembranol, such as HCl Form C, is characterized by a differential scanning calorimetry (DSC) thermogram according to FIG. 9B. In some embodiments, a solid HCl salt form of mesembranol, such as HCl Form C, is further characterized by a DSC having an endotherm at about 193° C. to about 197° C., and/or at about 97° C. to about 102° C., and/or at about 143° C. to about 148° C. (peak temperature), as measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

In some embodiments, the molar ratio of residual solvent EtOH/API is 0.04 (0.6 wt %), as measured by ¹H NMR. In some embodiments, the stoichiometric ratio of Cl⁻/freeform is about 0.8. In some embodiments, the purity of HCl Form C is about 99.3 area %, as measured by UPLC.

### Phosphate Form A

In some embodiments, solid forms of a phosphate salt of Compound 1 are provided. Phosphate Form A is a solid form of the phosphate salt of Compound 1 that can be identified by a XRPD pattern comprising certain characteristic peaks, and/or by one or more other techniques including DSC, TGA, and/or DVS.

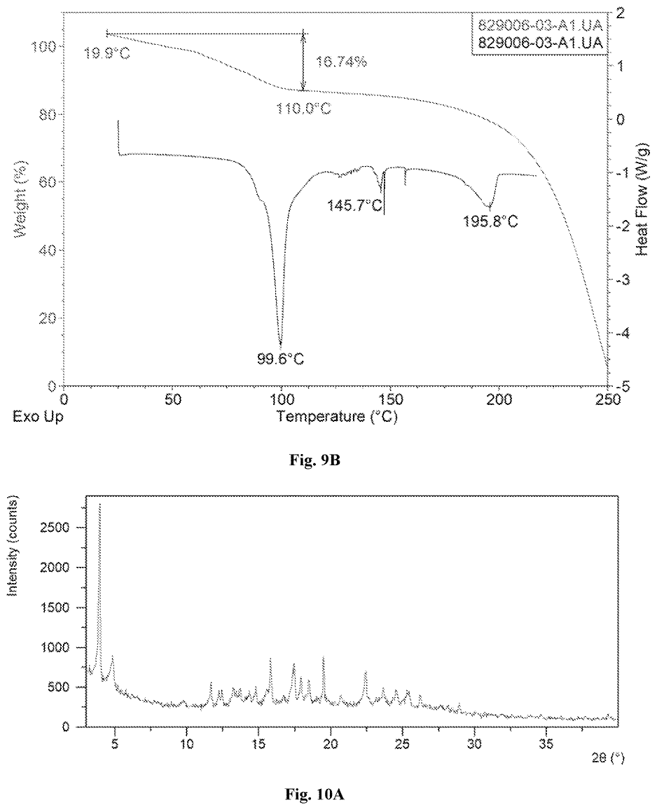

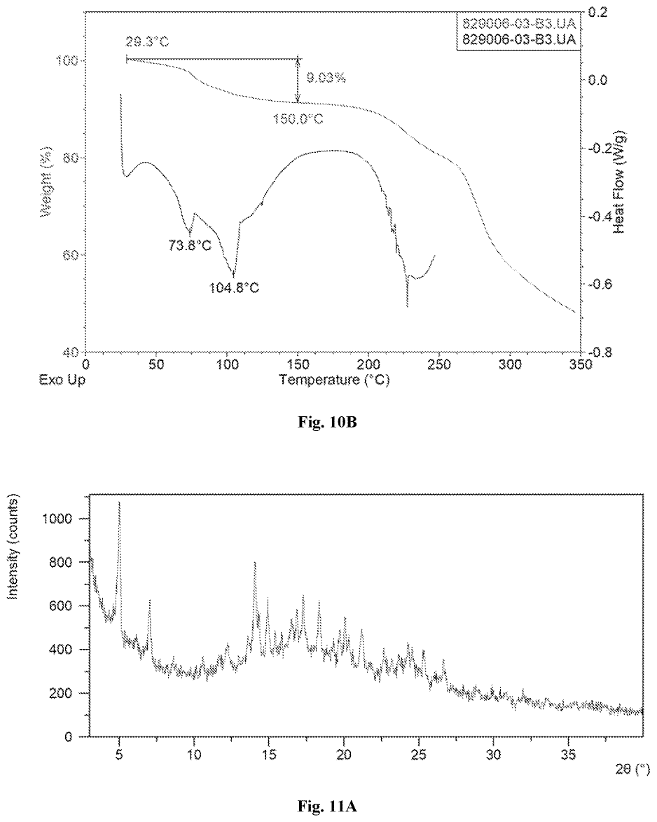

Samples of Phosphate Form A were prepared according to the Example 9 and characterized by XRPD (e.g., a pattern the same or substantially similar to or not dissimilar to FIG. 10A), TGA (e.g., a thermogram the same or substantially similar to or not dissimilar to FIG. 10B), and DSC (e.g., a thermogram the same or substantially similar to or not dissimilar to FIG. 10B).

In some embodiments, a solid phosphate salt form of mesembranol, such as Phosphate Form A, has an X-ray powder diffraction (XRPD) pattern comprising 2-theta peaks (2 theta±0.2) at 3.9°, 15.8°, 17.5°, 19.5°, and 22.4°. In some embodiments, a solid Phosphate salt form of mesembranol, such as Phosphate Form A, has an X-ray powder diffraction (XRPD) pattern comprising 2-theta peaks (2 theta±0.2) at 3.9°, 4.9°, 11.7°, 15.8°, 17.5°, 17.9°, 18.5°, 19.5°, 22.4°, and 24.6°.

In some embodiments, a solid phosphate salt form of mesembranol, such as Phosphate Form A, has an X-ray powder diffraction (XRPD) pattern comprising 2-theta peaks according to Table 10 having a relative intensity of greater than 10%. In some embodiments, a solid phosphate salt form of mesembranol, such as Phosphate Form A, has an X-ray powder diffraction (XRPD) pattern according to FIG. 10A.

In some embodiments, a solid phosphate salt form of mesembranol, such as Phosphate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 3.9 | 22.4 |
| 15.8 | 5.6 |
| 17.5 | 5.1 |
| 19.5 | 4.6 |
| 22.4 | 4.0. |

In some embodiments, a solid phosphate salt form of mesembranol, such as Phosphate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 3.9 | 22.4 |
| 4.9 | 18.2 |
| 11.7 | 7.6 |
| 15.8 | 5.6 |
| 17.5 | 5.1 |
| 17.9 | 5.0 |
| 18.5 | 4.8 |
| 19.5 | 4.6 |
| 22.4 | 4.0 |
| 24.6 | 3.6. |

In some embodiments, a solid phosphate salt form of mesembranol, such as Phosphate Form A, is characterized by a TGA thermogram according to FIG. 10B. In some embodiments, a solid phosphate salt form of mesembranol, such as Phosphate Form A, is further characterized by a sample weight loss of up to about 10% upon heating up to 150° C., as measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

In some embodiments, a solid phosphate salt form of mesembranol, such as Phosphate Form A, is characterized by a differential scanning calorimetry (DSC) thermogram according to FIG. 10B. In some embodiments, a solid phosphate salt form of mesembranol, such as Phosphate Form A, is further characterized by a DSC having an endotherm at about 102° C. to about 107° C. and/or at about 71° C. to about 76° C. (peak temperature), as measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

In some embodiments, the molar ratio of acetone/API is about 0.5 (7.1 wt %), as measured by ¹H NMR. In some embodiments, the stoichiometric ratio of PO₄³⁻/freeform is about 1.1, as measured by UPLC/IC. In some embodiments, the purity of Phosphate Form A is about 98.8 area % by UPLC.

### Tartrate Form A

In some embodiments, solid forms of a tartrate salt of Compound 1 are provided. Tartrate Form A is a solid form of the tartrate salt of Compound 1 that can be identified by a XRPD pattern comprising certain characteristic peaks, and/or by one or more other techniques including DSC, TGA, and/or DVS.

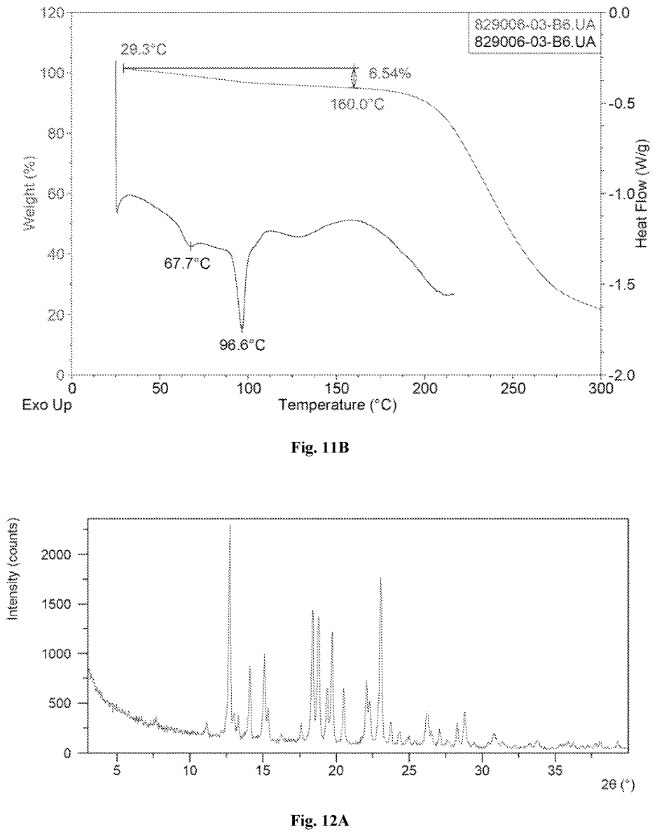

Samples of Tartrate Form A were prepared according to the Example 10 and characterized by XRPD (e.g., a pattern the same or substantially similar to or not dissimilar to FIG. 11A), TGA (e.g., a thermogram the same or substantially similar to or not dissimilar to FIG. 11B), and DSC (e.g., a thermogram the same or substantially similar to or not dissimilar to FIG. 11B).

In some embodiments, a solid tartrate salt form of mesembranol, such as Tartrate Form A, has an X-ray powder diffraction (XRPD) pattern comprising 2-theta peaks (2 theta±0.2) at 5.0°, 14.1°, 14.9°, 17.3°, and 18.4°. In some embodiments, a solid Tartrate salt form of mesembranol, such as Tartrate Form A, has an X-ray powder diffraction (XRPD) pattern comprising 2-theta peaks (2 theta±0.2) at 5.0°, 7.0°, 12.2°, 14.1°, 14.9°, 17.3°, 18.4°, 19.7°, 20.2°, 21.2°, 22.7°, 24.3°, 25.3°, and 26.7°.

In some embodiments, a solid tartrate salt form of mesembranol, such as Tartrate Form A, has an X-ray powder diffraction (XRPD) pattern comprising 2-theta peaks according to Table 11 having a relative intensity of greater than 10%. In some embodiments, a solid tartrate salt form of mesembranol, such as Tartrate Form A, has an X-ray powder diffraction (XRPD) pattern according to FIG. 11A.

In some embodiments, a solid tartrate salt form of mesembranol, such as Tartrate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 5.0 | 17.6 |
| 14.1 | 6.3 |
| 14.9 | 5.9 |
| 17.3 | 5.1 |
| 18.4 | 4.8. |

In some embodiments, a solid tartrate salt form of mesembranol, such as Tartrate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 5.0 | 17.6 |
| 7.0 | 12.6 |
| 12.2 | 7.2 |
| 14.1 | 6.3 |
| 14.9 | 5.9 |
| 17.3 | 5.1 |
| 18.4 | 4.8 |
| 19.7 | 4.5 |
| 20.2 | 4.4 |
| 21.2 | 4.2 |
| 22.7 | 3.9 |
| 24.3 | 3.7 |
| 25.3 | 3.5 |
| 26.7 | 3.3. |

In some embodiments, a solid tartrate salt form of mesembranol, such as Tartrate Form A, is characterized by a TGA thermogram according to FIG. 11B. In some embodiments, a solid tartrate salt form of mesembranol, such as Tartrate Form A, is further characterized by a sample weight loss of up to about 7% upon heating up to 160° C., as measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

In some embodiments, a solid tartrate salt form of mesembranol, such as Tartrate Form A, is characterized by a differential scanning calorimetry (DSC) thermogram according to FIG. 11B. In some embodiments, a solid tartrate salt form of mesembranol, such as Tartrate Form A, is further characterized by a DSC having an endotherm at about 94° C. to about 99° C. and/or at about 65° C. to about 70° C. (peak temperature), as measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

In some embodiments, the molar ratio of L-tartaric acid/freeform is about 1.2, as measured by ¹H NMR. In some embodiments, the residual solvent acetone/API is 0.2 (2.9 wt %), as measured by ¹H NMR. In some embodiments, the purity of Tartrate Form A is about 98.6 area %, as measured by UPLC.

### Fumarate Form A

In some embodiments, solid forms of a fumarate salt of Compound 1 are provided. Fumarate Form A is a solid form of the fumarate salt of Compound 1 that can be identified by a XRPD pattern comprising certain characteristic peaks, and/or by one or more other techniques including DSC, TGA, and/or DVS.

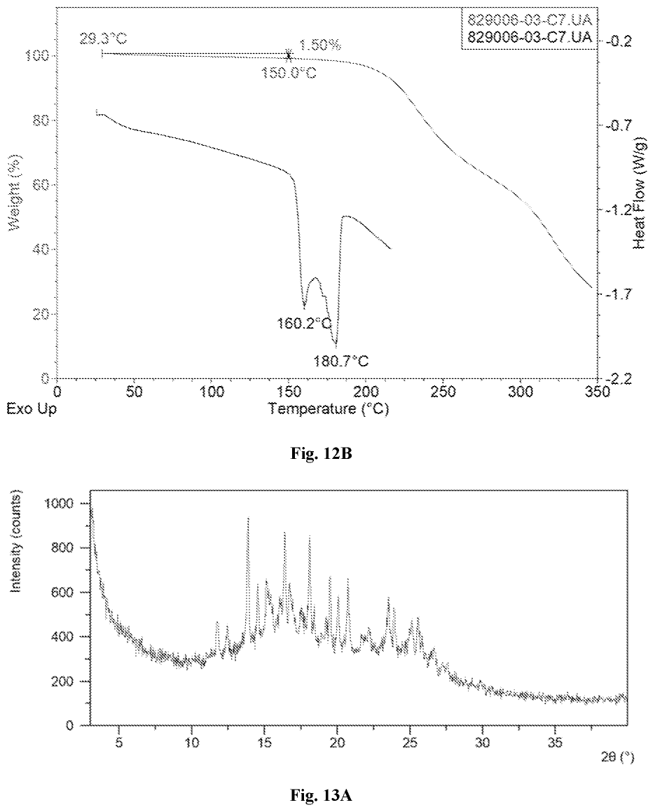

Samples of Fumarate Form A were prepared according to the Example 11 and characterized by XRPD (e.g., a pattern the same or substantially similar to or not dissimilar to FIG. 12A), TGA (e.g., a thermogram the same or substantially similar to or not dissimilar to FIG. 12B), and DSC (e.g., a thermogram the same or substantially similar to or not dissimilar to FIG. 12B).

In some embodiments, a solid fumarate salt form of mesembranol, such as Fumarate Form A, has an X-ray powder diffraction (XRPD) pattern comprising 2-theta peaks (2 theta±0.2) at 12.7°, 18.4°, 18.8°, 19.7°, and 23.1°. In some embodiments, a solid Fumarate salt form of mesembranol, such as Fumarate Form A, has an X-ray powder diffraction (XRPD) pattern comprising 2-theta peaks (2 theta±0.2) at 12.7°, 13.0°, 13.3°, 14.1°, 15.1°, 15.4°, 18.4°, 18.8°, 19.4°, 19.7°, 20.5°, 22.1°, 22.3°, 23.1°, 23.8°, 26.2°, 28.3°, and 28.8°.

In some embodiments, a solid fumarate salt form of mesembranol, such as Fumarate Form A, has an X-ray powder diffraction (XRPD) pattern comprising 2-theta peaks according to Table 12 having a relative intensity of greater than 10%. In some embodiments, a solid fumarate salt form of mesembranol, such as Fumarate Form A, has an X-ray powder diffraction (XRPD) pattern according to FIG. 12A.

In some embodiments, a solid fumarate salt form of mesembranol, such as Fumarate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 12.7 | 7.0 |
| 18.4 | 4.8 |
| 18.8 | 4.7 |
| 19.7 | 4.5 |
| 23.1 | 3.9. |

In some embodiments, a solid fumarate salt form of mesembranol, such as Fumarate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 12.7 | 7.0 |
| 13.0 | 6.8 |
| 13.3 | 6.7 |
| 14.1 | 6.3 |
| 15.1 | 5.9 |
| 15.4 | 5.8 |
| 18.4 | 4.8 |
| 18.8 | 4.7 |
| 19.4 | 4.6 |
| 19.7 | 4.5 |
| 20.5 | 4.3 |
| 22.1 | 4.0 |
| 22.3 | 4.0 |
| 23.1 | 3.9 |
| 23.8 | 3.7 |
| 26.2 | 3.4 |
| 28.3 | 3.2 |
| 28.8 | 3.1. |

In some embodiments, a solid fumarate salt form of mesembranol, such as Fumarate Form A, is characterized by a TGA thermogram according to FIG. 12B. In some embodiments, a solid fumarate salt form of mesembranol, such as Fumarate Form A, is further characterized by a sample weight loss of up to about 2% upon heating up to 160° C., as measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

In some embodiments, a solid fumarate salt form of mesembranol, such as Fumarate Form A, is characterized by a differential scanning calorimetry (DSC) thermogram according to FIG. 12B. In some embodiments, a solid fumarate salt form of mesembranol, such as Fumarate Form A, is further characterized by a DSC having an endotherm at about 178° C. to about 183° C. and/or at about 158° C. to about 163° C. (peak temperature), as measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

In some embodiments, the molar ratio of acid/API is about 1.2, as determined by 1H NMR. In some embodiments, the residual solvent EtOAc/API is about 0.04 (0.7 wt %), as determined by ¹H NMR. In some embodiments, the purity of Fumarate Form A is about 98.9 area %, as measured by UPLC.

### Naphthalene-1,5-Disulfonate Form A

In some embodiments, solid forms of a naphthalene-1,5-disulfonate salt of Compound 1 are provided. Naphthalene-1,5-disulfonate Form A is a solid form of the naphthalene-1,5-disulfonate salt of Compound 1 that can be identified by a XRPD pattern comprising certain characteristic peaks, and/or by one or more other techniques including DSC, TGA, and/or DVS.

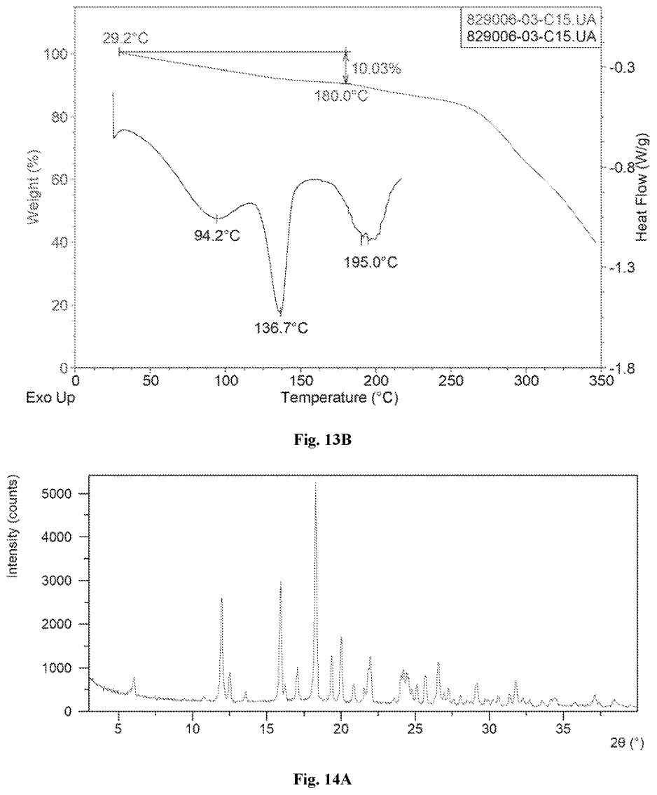

Samples of Naphthalene-1,5-disulfonate Form A were prepared according to the Example 12 and characterized by XRPD (e.g., a pattern the same or substantially similar to or not dissimilar to FIG. 13A), TGA (e.g., a thermogram the same or substantially similar to or not dissimilar to FIG. 13B), and DSC (e.g., a thermogram the same or substantially similar to or not dissimilar to FIG. 13B).

In some embodiments, a solid naphthalene-1,5-disulfonate salt form of mesembranol, such as Naphthalene-1,5-disulfonate Form A, has an X-ray powder diffraction (XRPD) pattern comprising 2-theta peaks (2 theta±0.2) at 13.9°, 15.2°, 16.4°, 18.1°, and 19.5°. In some embodiments, a solid Naphthalene-1,5-disulfonate salt form of mesembranol, such as Naphthalene-1,5-disulfonate Form A, has an X-ray powder diffraction (XRPD) pattern comprising 2-theta peaks (2 theta±0.2) at 11.8°, 12.4°, 13.9°, 14.5°, 15.2°, 16.4°, 18.1°, 19.5°, 20.1°, 20.7°, 22.0°, 23.5°, 23.9°, 25.2°, and 25.6°.

In some embodiments, a solid naphthalene-1,5-disulfonate salt form of mesembranol, such as Naphthalene-1,5-disulfonate Form A, has an X-ray powder diffraction (XRPD) pattern comprising 2-theta peaks according to Table 13 having a relative intensity of greater than 10%. In some embodiments, a solid naphthalene-1,5-disulfonate salt form of mesembranol, such as Naphthalene-1,5-disulfonate Form A, has an X-ray powder diffraction (XRPD) pattern according to FIG. 13A.

In some embodiments, a solid naphthalene-1,5-disulfonate salt form of mesembranol, such as Naphthalene-1,5-disulfonate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 13.9 | 6.4 |
| 15.2 | 5.8 |
| 16.4 | 5.4 |
| 18.1 | 4.9 |
| 19.5 | 4.6. |

In some embodiments, a solid naphthalene-1,5-disulfonate salt form of mesembranol, such as Naphthalene-1,5-disulfonate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 11.8 | 7.5 |
| 12.4 | 7.1 |
| 13.9 | 6.4 |
| 14.5 | 6.1 |
| 15.2 | 5.8 |
| 16.4 | 5.4 |
| 18.1 | 4.9 |
| 19.5 | 4.6 |
| 20.1 | 4.4 |
| 20.7 | 4.3 |
| 22.0 | 4.0 |
| 23.5 | 3.8 |
| 23.9 | 3.7 |
| 25.2 | 3.5 |
| 25.6 | 3.5. |

In some embodiments, a solid naphthalene-1,5-disulfonate salt form of mesembranol, such as Naphthalene-1,5-disulfonate Form A, is characterized by a TGA thermogram according to FIG. 13B. In some embodiments, a solid naphthalene-1,5-disulfonate salt form of mesembranol, such as Naphthalene-1,5-disulfonate Form A, is further characterized by a sample weight loss of up to about 11% upon heating up to 180° C., as measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

In some embodiments, a solid naphthalene-1,5-disulfonate salt form of mesembranol, such as Naphthalene-1,5-disulfonate Form A, is characterized by a differential scanning calorimetry (DSC) thermogram according to FIG. 13B. In some embodiments, a solid naphthalene-1,5-disulfonate salt form of mesembranol, such as Naphthalene-1,5-disulfonate Form A, is further characterized by a DSC having an endotherm at about 193° C. to about 198° C., and/or at about 92° C. to about 97° C., and/or at about 134° C. to about 139° C. (peak temperature), as measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

In some embodiments, the molar ratio of acid/API is about 1.8, as determined by 1H NMR. In some embodiments, the residual solvent EtOAc/API is about 0.07 (0.7 wt %), as determined by ¹H NMR. In some embodiments, the purity of Fumarate Form A is about 94.5 area %, as measured by UPLC.

### Oxalate Form A

In some embodiments, solid forms of an oxalate salt of Compound 1 are provided. Oxalate Form A is a solid form of the oxalate salt of Compound 1 that can be identified by a XRPD pattern comprising certain characteristic peaks, and/or by one or more other techniques including DSC, TGA, and/or DVS.

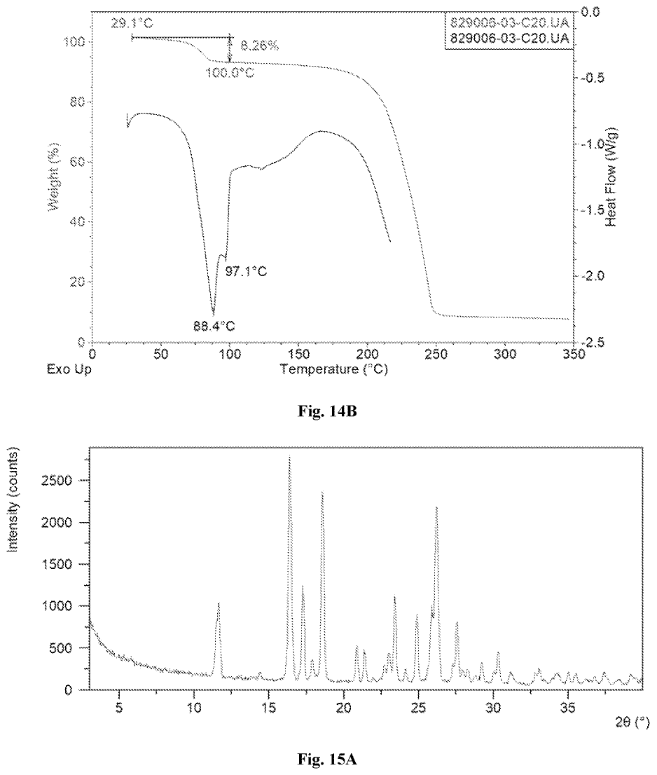

Samples of Oxalate Form A were prepared according to the Example 13 and characterized by XRPD (e.g., a pattern the same or substantially similar to or not dissimilar to FIG. 14A), TGA (e.g., a thermogram the same or substantially similar to or not dissimilar to FIG. 14B), and DSC (e.g., a thermogram the same or substantially similar to or not dissimilar to FIG. 14B).

In some embodiments, a solid oxalate salt form of mesembranol, such as Oxalate Form A, has an X-ray powder diffraction (XRPD) pattern comprising 2-theta peaks (2 theta±0.2) at 12.0°, 15.9°, 18.3°, 19.4°, and 20.0°. In some embodiments, a solid Oxalate salt form of mesembranol, such as Oxalate Form A, has an X-ray powder diffraction (XRPD) pattern comprising 2-theta peaks (2 theta±0.2) at 12.0°, 12.5°, 15.9°, 17.1°, 18.3°, 19.4°, 20.0°, 22.0°, 24.0°, 24.2°, 25.7°, 26.6°, 29.2°, and 31.8°.

In some embodiments, a solid oxalate salt form of mesembranol, such as Oxalate Form A, has an X-ray powder diffraction (XRPD) pattern comprising 2-theta peaks according to Table 14 having a relative intensity of greater than 10%. In some embodiments, a solid oxalate salt form of mesembranol, such as Oxalate Form A, has an X-ray powder diffraction (XRPD) pattern according to FIG. 14A.

In some embodiments, a solid oxalate salt form of mesembranol, such as Oxalate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 12.0 | 7.4 |
| 15.9 | 5.6 |
| 18.3 | 4.8 |
| 19.4 | 4.6 |
| 20.0 | 4.4. |

In some embodiments, a solid oxalate salt form of mesembranol, such as Oxalate Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 12.0 | 7.4 |
| 12.5 | 7.1 |
| 15.9 | 5.6 |
| 17.1 | 5.2 |
| 18.3 | 4.8 |
| 19.4 | 4.6 |
| 20.0 | 4.4 |
| 22.0 | 4.0 |
| 24.0 | 3.7 |
| 24.2 | 3.7 |
| 25.7 | 3.5 |
| 26.6 | 3.4 |
| 29.2 | 3.1 |
| 31.8 | 2.8. |

In some embodiments, a solid oxalate salt form of mesembranol, such as Oxalate Form A, is characterized by a TGA thermogram according to FIG. 14B. In some embodiments, a solid oxalate salt form of mesembranol, such as Oxalate Form A, is further characterized by a sample weight loss of up to about 9% upon heating up to 100° C., as measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

In some embodiments, a solid oxalate salt form of mesembranol, such as Oxalate Form A, is characterized by a differential scanning calorimetry (DSC) thermogram according to FIG. 14B. In some embodiments, a solid oxalate salt form of mesembranol, such as Oxalate Form A, is further characterized by a DSC having an endotherm at about 95° C. to about 100° C. and/or at about 86° C. to about 91° C. (peak temperature), as measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

In some embodiments, the stoichiometric ratio of C₂O₄²⁻/freeform is about 0.6, as measured by UPLC/IC. In some embodiments, the residual solvent EtOAc/API is about 0.1 (2.2 wt %), as determined by ¹H NMR. In some embodiments, the purity of Oxalate Form A is about 98.2 area %, as measured by UPLC.

### HBr Form A

In some embodiments, solid forms of an HBr salt of Compound 1 are provided. HBr Form A is a solid form of the HBr salt of Compound 1 that can be identified by a XRPD pattern comprising certain characteristic peaks, and/or by one or more other techniques including DSC, TGA, and/or DVS.

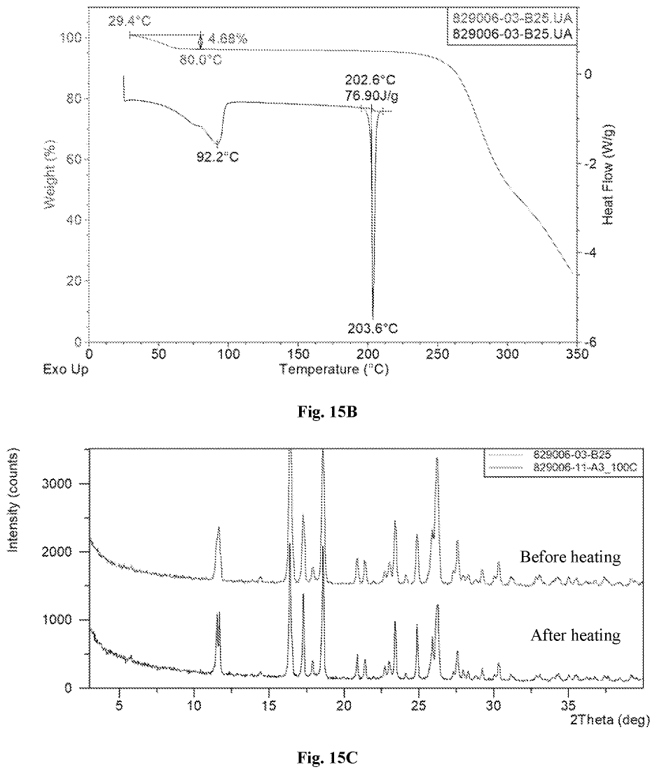

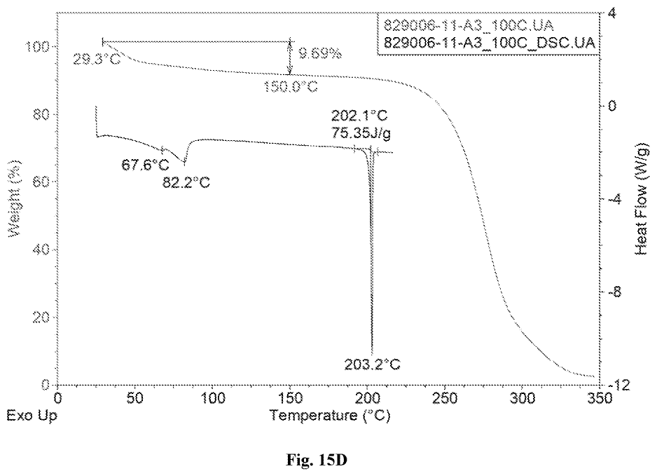

Samples of HBr Form A were prepared according to the Example 14 and characterized by XRPD (e.g., a pattern the same or substantially similar to or not dissimilar to FIG. 15A), TGA (e.g., a thermogram the same or substantially similar to or not dissimilar to FIG. 15B), and DSC (e.g., a thermogram the same or substantially similar to or not dissimilar to FIG. 15B).

In some embodiments, a solid HBr salt form of mesembranol, such as HBr Form A, has an X-ray powder diffraction (XRPD) pattern comprising 2-theta peaks (2 theta±0.2) at 16.4°, 17.2°, 18.6°, 23.4°, and 26.2°. In some embodiments, a solid HBr salt form of mesembranol, such as HBr Form A, has an X-ray powder diffraction (XRPD) pattern comprising 2-theta peaks (2 theta±0.2) at 11.6°, 16.4°, 17.2°, 17.9°, 18.6°, 20.9°, 21.4°, 23.0°, 23.4°, 24.9°, 25.9°, 26.2°, 27.6°, and 30.3°.

In some embodiments, a solid HBr salt form of mesembranol, such as HBr Form A, has an X-ray powder diffraction (XRPD) pattern comprising 2-theta peaks according to Table 15 having a relative intensity of greater than 10%. In some embodiments, a solid HBr salt form of mesembranol, such as HBr Form A, has an X-ray powder diffraction (XRPD) pattern according to FIG. 15A.

In some embodiments, a solid HBr salt form of mesembranol, such as HBr Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 16.4 | 5.4 |
| 17.2 | 5.1 |
| 18.6 | 4.8 |
| 23.4 | 3.8 |
| 26.2 | 3.4. |

In some embodiments, a solid HBr salt form of mesembranol, such as HBr Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 11.6 | 7.6 |
| 16.4 | 5.4 |
| 17.2 | 5.1 |
| 17.9 | 5.0 |
| 18.6 | 4.8 |
| 20.9 | 4.3 |
| 21.4 | 4.2 |
| 23.0 | 3.9 |
| 23.4 | 3.8 |
| 24.9 | 3.6 |
| 25.9 | 3.4 |
| 26.2 | 3.4 |
| 27.6 | 3.2 |
| 30.3 | 3.0. |

In some embodiments, a solid HBr salt form of mesembranol, such as HBr Form A, is characterized by a TGA thermogram according to FIG. 15B. In some embodiments, a solid HBr salt form of mesembranol, such as HBr Form A, is further characterized by a sample weight loss of up to about 5% upon heating to 80° C., as measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

In some embodiments, a solid HBr salt form of mesembranol, such as HBr Form A, is characterized by a differential scanning calorimetry (DSC) thermogram according to FIG. 15B. In some embodiments, a solid HBr salt form of mesembranol, such as HBr Form A, is further characterized by a DSC having an endotherm at about 200° C. to about 205° C. and/or at about 90° C. to about 95° C. (peak temperature), as measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

In some embodiments, the stoichiometric ratio of Br/freeform is about 1.0, as measured by UPLC/IC. In some embodiments, no residual solvent is detected, as determined by ¹H NMR. In some embodiments, the purity of HBr Form A is about 99.8 area %, as measured by UPLC.

### Pharmaceutical Compositions & Methods of Treatment

In some embodiments, pharmaceutical compositions comprising mesembranol, and pharmaceutically acceptable salts and hydrates thereof are provided. The pharmaceutical composition can comprise mesembranol in one or more solid forms provided herein, such as crystalline mesembranol in a hydrated or anhydrous solid form. A pharmaceutical composition, as used herein, refers to a mixture of mesembranol optionally further comprising other pharmaceutically acceptable components, such as carriers, stabilizers, diluents, dispersing agents, suspending agents, thickening agents, and/or excipients. The pharmaceutical composition can facilitate administration of the compound to a mammal, including compositions formulated for oral administration of mesembranol to a mammal (e.g., capsules or tablets).

In some embodiments, crystalline mesembranol is incorporated into pharmaceutical compositions to provide solid oral dosage forms. In other embodiments, crystalline mesembranol is used to prepare pharmaceutical compositions prepared for oral solid dosage forms. In some embodiments, the pharmaceutical composition comprises an active pharmaceutical ingredient (API) comprising, consisting essentially of, or consisting of mesembranol prepared under applicable Good Manufacturing Practice (GMP). For example, the pharmaceutical composition can be a batch composition comprising mesembranol, wherein the batch composition adheres to Good Manufacturing Practices (e.g., ICH Harmonised Tripartite Guideline, Good Manufacturing Practice Guide for Active Pharmaceutical Ingredients Q7, Current Step 4 version dated 10 Nov. 2010). More preferably, the GMP batch composition can be a homogenous blended batch comprising mesembranol. The FDA (Food and Drug Administration) provides applicable guidance on Good Manufacturing Practice (GMP) for the manufacturing of active pharmaceutical ingredients (APIs) under an appropriate system for managing quality. As used with respect to manufacture of API under GMP, “manufacturing” is defined to include all operations of receipt of materials, production, packaging, repackaging, labelling, relabeling, quality control, release, storage and distribution of APIs and the related controls. An “API Starting Material” is a raw material, intermediate, or an API that is used in the production of an API and that is incorporated as a significant structural fragment into the structure of the API. An API Starting Material can be an article of commerce, a material purchased from one or more suppliers under contract or commercial agreement, or produced in-house. API Starting Materials normally have defined chemical properties and structure.

The pharmaceutical compositions comprising mesembranol can be administered to patients in need thereof, to provide a therapeutically effective amount of a compound of mesembranol.

In practicing the methods of treatment or use provided herein, therapeutically effective amounts of mesembranol are administered in a pharmaceutical composition to a mammal having a disease, disorder, or condition to be treated. In some embodiments, the disease, disorder, or condition is a central nervous system disorder or an inflammatory condition. In some embodiments, pharmaceutical compositions reported herein can be provided in a unit dosage form container (e.g., in a vial or bag or the like).

In some embodiments, methods of treating a patient suffering from a disease comprise administering to a patient a composition comprising a compound disclosed herein for the treatment or prevention of a mental health disorder. In some embodiments, methods of treating a patient suffering from a disease comprise administering to a patient a composition comprising a compound disclosed herein for the treatment or prevention of a diagnosed condition selected from anxiety and depression. In some embodiments, the compound disclosed herein is administered to the patient in a unit dose. In some embodiments, a method comprises the administration to a patient in need thereof of a therapeutically effective amount of a mesembranol composition for the treatment of a disease selected from the group consisting of mild to moderate depression and major depressive episodes. In some embodiments, a method comprises the administration to a patient in need thereof of a therapeutically effective amount of a mesembranol composition for the treatment of anxiety. In some embodiments, a method comprises the administration to a patient in need thereof of a therapeutically effective amount of a mesembranol composition for the treatment of depression. In some embodiments, a method comprises the administration to a patient in need thereof of a therapeutically effective amount of a mesembranol composition for the treatment of a condition selected from the group consisting of: anxiety associated with depression, anxiety with depression, mixed anxiety and depressive disorder. In some embodiments, a method comprises the administration to a patient in need thereof of a therapeutically effective amount of a mesembranol composition for the treatment of anxiety and hysteria or anxiety and depression.

In some embodiments, pharmaceutical compositions reported herein can be provided in an oral dosage form. In some embodiments, an oral dosage form of mesembranol can be a capsule. In some embodiments, an oral dosage form of mesembranol is a tablet. In some embodiments, an oral dosage form comprises a filler. In some embodiments, an oral dosage form comprises two fillers. In some embodiments, an oral dosage form comprises one or more fillers. In some embodiments, an oral dosage form comprises one or more disintegrants. In some embodiments, the oral dosage form comprises one or more lubricants. In some embodiments, an oral dosage form comprises one or more glidants, anti-adherents and/or anti-statics. In some embodiments, an oral dosage form is prepared via dry blending. In some embodiments, an oral dosage form is a tablet and is prepared via dry granulation.

In some embodiments, a pharmaceutical composition comprises a solid form described herein; and a pharmaceutically acceptable excipient. In some embodiments, a pharmaceutical composition is formed by a process comprising dissolving a solid form described herein.

In some embodiments, a method of treating a mental health disorder comprises administering to a mammal in need thereof an effective amount of a solid form described herein or a pharmaceutical composition described herein. In some embodiments, the mental health disorder is anxiety, stress, or depression. In some embodiments, the mammal is a human.

In some embodiments, a method of treating a mental health disorder comprises administering to a mammal in need thereof an effective amount of a solid form of (−) mesembranol selected from the group consisting of:
a.) Sulfate Form A having an X-ray powder diffraction (XRPD) pattern comprising a 2-theta peak (2-theta±0.2) at 16.1°;
b.) Malate Form A having an X-ray powder diffraction (XRPD) pattern comprising a 2-theta peak (2-theta±0.2) at 13.1°;
c.) Malate Form B having an X-ray powder diffraction (XRPD) pattern comprising a 2-theta peak (2-theta±0.2) at 18.1;
d.) Gentisate Form A having an X-ray powder diffraction (XRPD) pattern comprising a 2-theta peak (2-theta±0.2) at 16.6°;
e.) Maleate Form A having an X-ray powder diffraction (XRPD) pattern comprising a 2-theta peak (2-theta±0.2) at 14.2°;
f.) HCl Form A having an X-ray powder diffraction (XRPD) pattern comprising a 2-theta peak (2-theta±0.2) at 16.6°;
g.) HCl Form B having an X-ray powder diffraction (XRPD) pattern comprising a 2-theta peak (2-theta±0.2) at 16.8°;
h.) HCl Form C having an X-ray powder diffraction (XRPD) pattern comprising a 2-theta peak (2-theta±0.2) at 17.9°;
i.) Phosphate Form A having an X-ray powder diffraction (XRPD) pattern comprising a 2-theta peak (2-theta±0.2) at 3.9°;
j.) Tartrate Form A having an X-ray powder diffraction (XRPD) pattern comprising a 2-theta peak (2-theta±0.2) at 5.0°;
k.) Fumarate Form A having an X-ray powder diffraction (XRPD) pattern comprising a 2-theta peak (2-theta±0.2) at 12.7°;
l.) Napthalene-1,5-disulfonate Form A having an X-ray powder diffraction (XRPD) pattern comprising a 2-theta peak (2-theta±0.2) at 13.9°;
m.) Oxalate Form A having an X-ray powder diffraction (XRPD) pattern comprising a 2-theta peak (2-theta±0.2) at 18.3°; and
n.) HBr Form A having an X-ray powder diffraction (XRPD) pattern comprising a 2-theta peak (2-theta±0.2) at 16.4°.

In some embodiments, the recited XRPD peaks for each solid form are the most intense peaks by relative intensity in the respective XRPD patterns.

In some embodiments, a method of treating a mental health disorder comprises administering to a mammal in need thereof an effective amount of a solid form of (−) mesembranol selected from the group consisting of:
a.) Sulfate Form A having an X-ray powder diffraction (XRPD) pattern comprising a 2-theta peak (2-theta±0.2) at 16.1°;
b.) Malate Form A having an X-ray powder diffraction (XRPD) pattern comprising a 2-theta peak (2-theta±0.2) at 13.1°;
c.) Malate Form B having an X-ray powder diffraction (XRPD) pattern comprising a 2-theta peak (2-theta±0.2) at 20.2;
d.) Gentisate Form A having an X-ray powder diffraction (XRPD) pattern comprising a 2-theta peak (2-theta±0.2) at 16.6°;
e.) Maleate Form A having an X-ray powder diffraction (XRPD) pattern comprising a 2-theta peak (2-theta±0.2) at 14.2°;
f.) HCl Form A having an X-ray powder diffraction (XRPD) pattern comprising a 2-theta peak (2-theta±0.2) at 18.5°;
g.) HCl Form B having an X-ray powder diffraction (XRPD) pattern comprising a 2-theta peak (2-theta±0.2) at 19.8°;
h.) HCl Form C having an X-ray powder diffraction (XRPD) pattern comprising a 2-theta peak (2-theta±0.2) at 9.0°;
i.) Phosphate Form A having an X-ray powder diffraction (XRPD) pattern comprising a 2-theta peak (2-theta±0.2) at 3.9°;
j.) Tartrate Form A having an X-ray powder diffraction (XRPD) pattern comprising a 2-theta peak (2-theta±0.2) at 5.0°;
k.) Fumarate Form A having an X-ray powder diffraction (XRPD) pattern comprising a 2-theta peak (2-theta±0.2) at 12.7°;
l.) Napthalene-1,5-disulfonate Form A having an X-ray powder diffraction (XRPD) pattern comprising a 2-theta peak (2-theta±0.2) at 18.1°;
m.) Oxalate Form A having an X-ray powder diffraction (XRPD) pattern comprising a 2-theta peak (2-theta±0.2) at 15.9°; and
n.) HBr Form A having an X-ray powder diffraction (XRPD) pattern comprising a 2-theta peak (2-theta±0.2) at 16.4°.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines), companion animals (e.g., canines, felines) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

### Additional Embodiments

1 Crystalline Sulfate Form A of mesembranol having an X-ray powder diffraction (XRPD) pattern comprising 2-theta peaks (2-theta±0.2) at 16.1°, 16.5°, 17.5°, 18.1°, and 18.7°.

2. The crystalline form of embodiment 1, having an X-ray powder diffraction (XRPD) pattern comprising 2-theta peaks (2-theta±0.2) at 11.2°, 11.7°, 13.0°, 13.9°, 14.5°, 15.8°, 16.1°, 16.5°, 16.9°, 17.5°, 18.1°, 18.7°, 20.6°, 21.8°, 23.4°, 24.6°, 24.7°, 25.3°, 25.8°, 26.0°, and 26.3°.

3. The crystalline form of embodiment 1, having an X-ray powder diffraction (XRPD) pattern comprising the 2-theta peaks in Table 2 having a relative intensity of greater than 10%.

4. The crystalline form of embodiment 1, having an X-ray powder diffraction (XRPD) pattern according to FIG. 2A.

5. The crystalline form of embodiment 1, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 16.1 | 5.5 |
| 16.5 | 5.4 |
| 17.5 | 5.1 |
| 18.1 | 4.9 |
| 18.7 | 4.8. |

6. The crystalline form of embodiment 1, characterized by an X-ray powder diffraction (XRPD) comprising the following 2-theta peaks (2-theta±0.2) and corresponding d-spacing

| 2θ[°] | d-spacing [Å] |
|---|---|
| 11.2 | 7.9 |
| 11.7 | 7.6 |
| 13.0 | 6.8 |
| 13.9 | 6.4 |
| 14.5 | 6.1 |
| 15.8 | 5.6 |
| 16.1 | 5.5 |
| 16.5 | 5.4 |
| 16.9 | 5.2 |
| 17.5 | 5.1 |
| 18.1 | 4.9 |
| 18.7 | 4.8 |
| 20.6 | 4.3 |
| 21.8 | 4.1 |
| 23.4 | 3.8 |
| 24.6 | 3.6 |
| 24.7 | 3.6 |
| 25.3 | 3.5 |
| 25.8 | 3.4 |
| 26.0 | 3.4 |
| 26.3 | 3.4. |

7. The crystalline form of any one of embodiments 1-6, characterized by a TGA thermogram according to FIG. 2B.

8. The crystalline form of any one of embodiments 1-6, further characterized by a sample weight loss of up to about 3.9% upon heating to 200° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

9. The crystalline form of any one of embodiments 1-8, characterized by a differential scanning calorimetry (DSC) thermogram according to FIG. 2B.

10. The crystalline form of any one of embodiments 1-8, further characterized by a DSC having an endotherm at about 157° C. to about 163° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

11. Crystalline Malate Form A of mesembranol having an X-ray powder diffraction (XRPD) pattern comprising 2-theta peaks (2-theta±0.2) at 13.1°, 16.0°, 20.8°, 21.5°, and 23.7°.

12. The crystalline form of embodiment 11, having an X-ray powder diffraction (XRPD) pattern comprising 2-theta peaks (2-theta±0.2) at 11.3°, 12.7°, 13.1°, 16.0°, 16.4°, 20.0°, 20.8°, 21.5°, 22.7°, 23.0°, 23.4°, 23.7°, 24.4°, 25.6°, 28.5°, and 31.0°.

13. The crystalline form of embodiment 11, having an X-ray powder diffraction (XRPD) pattern comprising the 2-theta peaks in Table 3 having a relative intensity of greater than 10%.

14. The crystalline form of embodiment 11, having an X-ray powder diffraction (XRPD) pattern according to FIG. 3A.

15. The crystalline form of embodiment 11, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 13.1 | 6.8 |
| 16.0 | 5.5 |
| 20.8 | 4.3 |
| 21.5 | 4.1 |
| 23.7 | 3.8. |

16. The crystalline form of embodiment 11, characterized by an X-ray powder diffraction (XRPD) comprising the following 2-theta peaks (2-theta±0.2) and corresponding d-spacing

| 2θ[°] | d-spacing [Å] |
|---|---|
| 11.3 | 7.8 |
| 12.7 | 7.0 |
| 13.1 | 6.8 |
| 16.0 | 5.5 |
| 16.4 | 5.4 |
| 20.0 | 4.4 |
| 20.8 | 4.3 |
| 21.5 | 4.1 |
| 22.7 | 3.9 |
| 23.0 | 3.9 |
| 23.4 | 3.8 |
| 23.7 | 3.8 |
| 24.4 | 3.6 |
| 25.6 | 3.5 |
| 28.5 | 3.1 |
| 31.0 | 2.9. |

17. The crystalline form of any one of embodiments 11-16, characterized by a TGA thermogram according to FIG. 3B.

18. The crystalline form of any one of embodiments 11-16, further characterized by a sample weight loss of up to about 1.9% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

19. The crystalline form of any one of embodiments 11-18, characterized by a differential scanning calorimetry (DSC) thermogram according to FIG. 3B.

20. The crystalline form of any one of embodiments 11-18, further characterized by a DSC having an endotherm at about 140° C. to about 145° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

21. Crystalline Malate Form B of mesembranol having an X-ray powder diffraction (XRPD) pattern comprising 2-theta peaks (2-theta±0.2) at 4.6°, 12.8°, 13.5°, 18.1°, and 21.6°.

22. The crystalline form of embodiment 21, having an X-ray powder diffraction (XRPD) pattern comprising 2-theta peaks (2-theta±0.2) at 4.6°, 9.0°, 12.5°, 12.8°, 13.5°, 16.4°, 16.9°, 18.1°, 19.0°, 19.7°, 19.9°, 20.2°, 21.6°, 24.7°, 26.5°, 27.7°, and 30.2°.

23. The crystalline form of embodiment 21, having an X-ray powder diffraction (XRPD) pattern comprising the 2-theta peaks in Table 4 having a relative intensity of greater than 10%.

24. The crystalline form of embodiment 21, having an X-ray powder diffraction (XRPD) pattern according to FIG. 4A.

25. The crystalline form of embodiment 21, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 4.6 | 19.4 |
| 12.8 | 6.9 |
| 13.5 | 6.5 |
| 18.1 | 4.9 |
| 21.6 | 4.1. |

26. The crystalline form of embodiment 21, characterized by an X-ray powder diffraction (XRPD) comprising the following 2-theta peaks (2-theta±0.2) and corresponding d-spacing

| 2θ[°] | d-spacing [Å] |
|---|---|
| 4.6 | 19.4 |
| 9.0 | 9.8 |
| 12.5 | 7.1 |
| 12.8 | 6.9 |
| 13.5 | 6.5 |
| 16.4 | 5.4 |
| 16.9 | 5.3 |
| 18.1 | 4.9 |
| 19.0 | 4.7 |
| 19.7 | 4.5 |
| 19.9 | 4.5 |
| 20.2 | 4.4 |
| 21.6 | 4.1 |
| 24.7 | 3.6 |
| 26.5 | 3.4 |
| 27.7 | 3.2 |
| 30.2 | 3.0. |

27. The crystalline form of any one of embodiments 21-26, characterized by a TGA thermogram according to FIG. 4B.

28. The crystalline form of any one of embodiments 21-26, further characterized by a sample weight loss of up to about 4.4% upon heating to 110° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

29. The crystalline form of any one of embodiments 21-28, characterized by a differential scanning calorimetry (DSC) thermogram according to FIG. 4B.

30. The crystalline form of any one of embodiments 21-28, further characterized by a DSC having an endotherm at about 99° C. to about 104° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

31. Crystalline Gentisate Form A of mesembranol having an X-ray powder diffraction (XRPD) pattern comprising 2-theta peaks (2-theta±0.2) at 9.0°, 16.6°, 17.1°, 19.0°, and 24.2°.

32. The crystalline form of embodiment 31, having an X-ray powder diffraction (XRPD) pattern comprising 2-theta peaks (2-theta±0.2) at 9.0°, 11.5°, 12.9°, 13.6°, 14.2°, 16.6°, 17.1°, 17.9°, 19.0°, 19.4°, 20.5°, 21.0°, 21.5°, 21.9°, 22.2°, 23.7°, 24.2°, and 25.2°.

33. The crystalline form of embodiment 31, having an X-ray powder diffraction (XRPD) pattern comprising the 2-theta peaks in Table 5 having a relative intensity of greater than 10%.

34. The crystalline form of embodiment 31, having an X-ray powder diffraction (XRPD) pattern according to FIG. 5A.

35. The crystalline form of embodiment 31, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 9.0 | 9.9 |
| 16.6 | 5.4 |
| 17.1 | 5.2 |
| 19.0 | 4.7 |
| 24.2 | 3.7. |

36. The crystalline form of embodiment 31, characterized by an X-ray powder diffraction (XRPD) comprising the following 2-theta peaks (2-theta±0.2) and corresponding d-spacing

| 2θ[°] | d-spacing [Å] |
|---|---|
| 9.0 | 9.9 |
| 11.5 | 7.7 |
| 12.9 | 6.9 |
| 13.6 | 6.5 |
| 14.2 | 6.3 |
| 16.6 | 5.4 |
| 17.1 | 5.2 |
| 17.9 | 5.0 |
| 19.0 | 4.7 |
| 19.4 | 4.6 |
| 20.5 | 4.3 |
| 21.0 | 4.2 |
| 21.5 | 4.1 |
| 21.9 | 4.0 |
| 22.2 | 4.0 |
| 23.7 | 3.8 |
| 24.2 | 3.7 |
| 25.2 | 3.5. |

37. The crystalline form of any one of embodiments 31-36, characterized by a TGA thermogram according to FIG. 5B.

38. The crystalline form of any one of embodiments 31-36, further characterized by a sample weight loss of up to about 2.1% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

39. The crystalline form of any one of embodiments 31-38, characterized by a differential scanning calorimetry (DSC) thermogram according to FIG. 5B.

40. The crystalline form of any one of embodiments 31-38, further characterized by a DSC having an endotherm at about 192° C. to about 197° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

41. Crystalline Maleate Form A of mesembranol having an X-ray powder diffraction (XRPD) pattern comprising 2-theta peaks (2-theta±0.2) at 14.2°, 15.6°, 16.4°, 19.9°, and 22.1°.

42. The crystalline form of embodiment 41, having an X-ray powder diffraction (XRPD) pattern comprising 2-theta peaks (2-theta±0.2) at 11.3°, 13.0°, 13.3°, 14.2°, 15.6°, 15.8°, 16.4°, 17.4°, 18.5°, 19.1°, 19.9°, 22.1°, 22.9°, 23.8°, 24.2°, 25.4°, 28.5°, and 29.5°.

43. The crystalline form of embodiment 41, having an X-ray powder diffraction (XRPD) pattern comprising the 2-theta peaks in Table 6 having a relative intensity of greater than 10%.

44. The crystalline form of embodiment 41, having an X-ray powder diffraction (XRPD) pattern according to FIG. 6A.

45. The crystalline form of embodiment 41, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 14.2 | 6.2 |
| 15.6 | 5.7 |
| 16.4 | 5.4 |
| 19.9 | 4.5 |
| 22.1 | 4.0. |

46. The crystalline form of embodiment 41, characterized by an X-ray powder diffraction (XRPD) comprising the following 2-theta peaks (2-theta±0.2) and corresponding d-spacing

| 2θ[°] | d-spacing [Å] |
|---|---|
| 11.3 | 7.8 |
| 13.0 | 6.8 |
| 13.3 | 6.7 |
| 14.2 | 6.2 |
| 15.6 | 5.7 |
| 15.8 | 5.6 |
| 16.4 | 5.4 |
| 17.4 | 5.1 |
| 18.5 | 4.8 |
| 19.1 | 4.6 |
| 19.9 | 4.5 |
| 22.1 | 4.0 |
| 22.9 | 3.9 |
| 23.8 | 3.7 |
| 24.2 | 3.7 |
| 25.4 | 3.5 |
| 28.5 | 3.1 |
| 29.5 | 3.0. |

47. The crystalline form of any one of embodiments 41-46, characterized by a TGA thermogram according to FIG. 6B.

48. The crystalline form of any one of embodiments 41-46, further characterized by a sample weight loss of up to about 1.3% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

49. The crystalline form of any one of embodiments 41-48, characterized by a differential scanning calorimetry (DSC) thermogram according to FIG. 6B.

50. The crystalline form of any one of embodiments 41-48, further characterized by a DSC having an endotherm at about 165° C. to about 170° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

51. Crystalline HCl Form A of mesembranol having an X-ray powder diffraction (XRPD) pattern comprising 2-theta peaks (2-theta±0.2) at 16.6°, 17.0°, 18.5°, 18.8°, and 26.7°.

52. The crystalline form of embodiment 51, having an X-ray powder diffraction (XRPD) pattern comprising 2-theta peaks (2-theta±0.2) at 11.4°, 11.7°, 13.2°, 16.6°, 17.0°, 17.7°, 18.5°, 18.8°, 20.9°, 23.5°, 24.9°, 25.8°, 26.0°, 26.2°, 26.7°, 27.6°, 28.1°, and 28.5°.

53. The crystalline form of embodiment 51, having an X-ray powder diffraction (XRPD) pattern comprising the 2-theta peaks in Table 7 having a relative intensity of greater than 10%.

54. The crystalline form of embodiment 51, having an X-ray powder diffraction (XRPD) pattern according to FIG. 7A.

55. The crystalline form of embodiment 51, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 16.6 | 5.3 |
| 17.0 | 5.2 |
| 18.5 | 4.8 |
| 18.8 | 4.7 |
| 26.7 | 3.3. |

56. The crystalline form of embodiment 51, characterized by an X-ray powder diffraction (XRPD) comprising the following 2-theta peaks (2-theta±0.2) and corresponding d-spacing

| 2θ[°] | d-spacing [Å] |
|---|---|
| 11.4 | 7.8 |
| 11.7 | 7.6 |
| 13.2 | 6.7 |
| 16.6 | 5.3 |
| 17.0 | 5.2 |
| 17.7 | 5.0 |
| 18.5 | 4.8 |
| 18.8 | 4.7 |
| 20.9 | 4.3 |
| 23.5 | 3.8 |
| 24.9 | 3.6 |
| 25.8 | 3.5 |
| 26.0 | 3.4 |
| 26.2 | 3.4 |
| 26.7 | 3.3 |
| 27.6 | 3.2 |
| 28.1 | 3.2 |
| 28.5 | 3.1. |

57. The crystalline form of any one of embodiments 51-56, characterized by a TGA thermogram according to FIG. 7B.

58. The crystalline form of any one of embodiments 51-56, further characterized by a sample weight loss of up to about 8.5% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

59. The crystalline form of any one of embodiments 51-58, characterized by a differential scanning calorimetry (DSC) thermogram according to FIG. 7B.

60. The crystalline form of any one of embodiments 51-58, further characterized by a DSC having an endotherm at about 197° C. to about 202° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

61. Crystalline HCl Form B of mesembranol having an X-ray powder diffraction (XRPD) pattern comprising 2-theta peaks (2-theta±0.2) at 13.4°, 16.8°, 18.2°, 19.6°, and 19.8°.

62. The crystalline form of embodiment 61, having an X-ray powder diffraction (XRPD) pattern comprising 2-theta peaks (2-theta±0.2) at 10.7°, 11.5°, 12.4°, 12.6°, 13.4°, 14.7°, 15.4°, 16.0°, 16.8°, 18.2°, 18.5°, 19.6°, 19.8°, 20.2°, 24.7°, 25.8°, 26.8°, 27.2°, 27.7°, and 28.4°. 63. The crystalline form of embodiment 61, having an X-ray powder diffraction (XRPD) pattern comprising the 2-theta peaks in Table 8 having a relative intensity of greater than 10%.

64. The crystalline form of embodiment 61, having an X-ray powder diffraction (XRPD) pattern according to FIG. 8A.

65. The crystalline form of embodiment 61, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 13.4 | 6.6 |
| 16.8 | 5.3 |
| 18.2 | 4.9 |
| 19.6 | 4.5 |
| 19.8 | 4.5. |

66. The crystalline form of embodiment 61, characterized by an X-ray powder diffraction (XRPD) comprising the following 2-theta peaks (2-theta±0.2) and corresponding d-spacing

| 2θ[°] | d-spacing [Å] |
|---|---|
| 10.7 | 8.3 |
| 11.5 | 7.7 |
| 12.4 | 7.2 |
| 12.6 | 7.0 |
| 13.4 | 6.6 |
| 14.7 | 6.0 |
| 15.4 | 5.7 |
| 16.0 | 5.5 |
| 16.8 | 5.3 |
| 18.2 | 4.9 |
| 18.5 | 4.8 |
| 19.6 | 4.5 |
| 19.8 | 4.5 |
| 20.2 | 4.4 |
| 24.7 | 3.6 |
| 25.8 | 3.5 |
| 26.8 | 3.3 |
| 27.2 | 3.3 |
| 27.7 | 3.2 |
| 28.4 | 3.2. |

67. The crystalline form of any one of embodiments 61-66, characterized by a TGA thermogram according to FIG. 8B.

68. The crystalline form of any one of embodiments 61-66, further characterized by a sample weight loss of up to about 12% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

69. The crystalline form of any one of embodiments 61-68, characterized by a differential scanning calorimetry (DSC) thermogram according to FIG. 8B.

70. The crystalline form of any one of embodiments 61-68, further characterized by a DSC having an endotherm at about 195° C. to about 200° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

71. Crystalline HCl Form C of mesembranol having an X-ray powder diffraction (XRPD) pattern comprising 2-theta peaks (2-theta±0.2) at 9.0°, 12.4°, 16.9°, 17.9°, and 26.1°.

72. The crystalline form of embodiment 71, having an X-ray powder diffraction (XRPD) pattern comprising 2-theta peaks (2-theta±0.2) at 9.0°, 10.4°, 12.4°, 16.9°, 17.1°, 17.9°, and 26.1°.

73. The crystalline form of embodiment 71, having an X-ray powder diffraction (XRPD) pattern comprising the 2-theta peaks in Table 9 having a relative intensity of greater than 10%.

74. The crystalline form of embodiment 71, having an X-ray powder diffraction (XRPD) pattern according to FIG. 9A.

75. The crystalline form of embodiment 71, characterized by an X-ray powder diffraction (XRPD) comprising the following 2-theta peaks (2-theta±0.2) and corresponding d-spacing

| 2θ[°] | d-spacing [Å] |
|---|---|
| 9.0 | 9.9 |
| 12.4 | 7.1 |
| 16.9 | 5.3 |
| 17.9 | 5.0 |
| 26.1 | 3.4. |

76. The crystalline form of embodiment 71, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 9.0 | 9.9 |
| 10.4 | 8.5 |
| 12.4 | 7.1 |
| 16.9 | 5.3 |
| 17.1 | 5.2 |
| 17.9 | 5.0 |
| 26.1 | 3.4. |

77. The crystalline form of any one of embodiments 71-76, characterized by a TGA thermogram according to FIG. 9B.

78. The crystalline form of any one of embodiments 71-76, further characterized by a sample weight loss of up to about 17% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

79. The crystalline form of any one of embodiments 71-78, characterized by a differential scanning calorimetry (DSC) thermogram according to FIG. 9B.

80. The crystalline form of any one of embodiments 71-78, further characterized by a DSC having an endotherm at about 193° C. to about 197° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

81. Crystalline Phosphate Form A of mesembranol having an X-ray powder diffraction (XRPD) pattern comprising 2-theta peaks (2-theta±0.2) at 3.9°, 15.8°, 17.5°, 19.5°, and 22.4°.

82. The crystalline form of embodiment 81, having an X-ray powder diffraction (XRPD) pattern comprising 2-theta peaks (2-theta±0.2) at 3.9°, 4.9°, 11.7°, 15.8°, 17.5°, 17.9°, 18.5°, 19.5°, 22.4°, and 24.6°.

83. The crystalline form of embodiment 81, having an X-ray powder diffraction (XRPD) pattern comprising the 2-theta peaks in Table 10 having a relative intensity of greater than 10%.

84. The crystalline form of embodiment 81, having an X-ray powder diffraction (XRPD) pattern according to FIG. 10A.

85. The crystalline form of embodiment 81, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 3.9 | 22.4 |
| 15.8 | 5.6 |
| 17.5 | 5.1 |
| 19.5 | 4.6 |
| 22.4 | 4.0. |

86. The crystalline form of embodiment 81, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 3.9 | 22.4 |
| 4.9 | 18.2 |
| 11.7 | 7.6 |
| 15.8 | 5.6 |
| 17.5 | 5.1 |
| 17.9 | 5.0 |
| 18.5 | 4.8 |
| 19.5 | 4.6 |
| 22.4 | 4.0 |
| 24.6 | 3.6. |

87. The crystalline form of any one of embodiments 81-86, characterized by a TGA thermogram according to FIG. 10B.

88. The crystalline form of any one of embodiments 81-86, further characterized by a sample weight loss of up to about 10% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

89. The crystalline form of any one of embodiments 81-88, characterized by a differential scanning calorimetry (DSC) thermogram according to FIG. 10B.

90. The crystalline form of any one of embodiments 81-88, further characterized by a DSC having an endotherm at about 102° C. to about 107° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

91. Crystalline Tartrate Form A of mesembranol having an X-ray powder diffraction (XRPD) pattern comprising 2-theta peaks (2-theta±0.2) at 5.0°, 14.1°, 14.9°, 17.3°, and 18.4°.

92. The crystalline form of embodiment 91, having an X-ray powder diffraction (XRPD) pattern comprising 2-theta peaks (2-theta±0.2) at 5.0°, 7.0°, 12.2°, 14.1°, 14.9°, 17.3°, 18.4°, 19.7°, 20.2°, 21.2°, 22.7°, 24.3°, 25.3°, and 26.7°.

93. The crystalline form of embodiment 91, having an X-ray powder diffraction (XRPD) pattern comprising the 2-theta peaks in Table 11 having a relative intensity of greater than 10%.

94. The crystalline form of embodiment 91, having an X-ray powder diffraction (XRPD) pattern according to FIG. 11A.

95. The crystalline form of embodiment 91, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 5.0 | 17.6 |
| 14.1 | 6.3 |
| 14.9 | 5.9 |
| 17.3 | 5.1 |
| 18.4 | 4.8. |

96. The crystalline form of embodiment 91, characterized by an X-ray powder diffraction (XRPD) comprising the following 2-theta peaks (2-theta±0.2) and corresponding d-spacing

| 2θ[°] | d-spacing [Å] |
|---|---|
| 5.0 | 17.6 |
| 7.0 | 12.6 |
| 12.2 | 7.2 |
| 14.1 | 6.3 |
| 14.9 | 5.9 |
| 17.3 | 5.1 |
| 18.4 | 4.8 |
| 19.7 | 4.5 |
| 20.2 | 4.4 |
| 21.2 | 4.2 |
| 22.7 | 3.9 |
| 24.3 | 3.7 |
| 25.3 | 3.5 |
| 26.7 | 3.3. |

97. The crystalline form of any one of embodiments 91-96, characterized by a TGA thermogram according to FIG. 11B.

98. The crystalline form of any one of embodiments 91-96, further characterized by a sample weight loss of up to about 7% upon heating to 160° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

99. The crystalline form of any one of embodiments 91-98, characterized by a differential scanning calorimetry (DSC) thermogram according to FIG. 11B.

100. The crystalline form of any one of embodiments 91-98, further characterized by a DSC having an endotherm at about 94° C. to about 99° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

101. Crystalline Fumarate Form A of mesembranol having an X-ray powder diffraction (XRPD) pattern comprising 2-theta peaks (2-theta±0.2) at 12.7°, 18.4°, 18.8°, 19.7°, and 23.1°.

102. The crystalline form of embodiment 101, having an X-ray powder diffraction (XRPD) pattern comprising 2-theta peaks (2-theta±0.2) at 12.7°, 13.0°, 13.3°, 14.1°, 15.1°, 15.4°, 18.4°, 18.8°, 19.4°, 19.7°, 20.5°, 22.1°, 22.3°, 23.1°, 23.8°, 26.2°, 28.3°, and 28.8°.

103. The crystalline form of embodiment 101, having an X-ray powder diffraction (XRPD) pattern comprising the 2-theta peaks in Table 12 having a relative intensity of greater than 10%,

104. The crystalline form of embodiment 101, having an X-ray powder diffraction (XRPD) pattern according to FIG. 12A.

105. The crystalline form of embodiment 101, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 12.7 | 7.0 |
| 18.4 | 4.8 |
| 18.8 | 4.7 |
| 19.7 | 4.5 |
| 23.1 | 3.9. |

106. The crystalline form of embodiment 101, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 12.7 | 7.0 |
| 13.0 | 6.8 |
| 13.3 | 6.7 |
| 14.1 | 6.3 |
| 15.1 | 5.9 |
| 15.4 | 5.8 |
| 18.4 | 4.8 |
| 18.8 | 4.7 |
| 19.4 | 4.6 |
| 19.7 | 4.5 |
| 20.5 | 4.3 |
| 22.1 | 4.0 |
| 22.3 | 4.0 |
| 23.1 | 3.9 |
| 23.8 | 3.7 |
| 26.2 | 3.4 |
| 28.3 | 3.2 |
| 28.8 | 3.1. |

107. The crystalline form of any one of embodiments 101-106, characterized by a TGA thermogram according to FIG. 12B.

108. The crystalline form of any one of embodiments 101-106, further characterized by a sample weight loss of up to about 2% upon heating to 160° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

109. The crystalline form of any one of embodiments 101-108, characterized by a differential scanning calorimetry (DSC) thermogram according to FIG. 12B.

110. The crystalline form of any one of embodiments 101-108, further characterized by a DSC having an endotherm at about 178° C. to about 183° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

111. Crystalline Naphthalene-1,5-disulfonate Form A of mesembranol having an X-ray powder diffraction (XRPD) pattern comprising 2-theta peaks (2-theta±0.2) at 13.9°, 15.2°, 16.4°, 18.1°, and 19.5°.

112. The crystalline form of embodiment 111, having an X-ray powder diffraction (XRPD) pattern comprising 2-theta peaks (2-theta±0.2) at 11.8°, 12.4°, 13.9°, 14.5°, 15.2°, 16.4°, 18.1°, 19.5°, 20.1°, 20.7°, 22.0°, 23.5°, 23.9°, 25.2°, and 25.6°.

113. The crystalline form of embodiment 111, having an X-ray powder diffraction (XRPD) pattern comprising the 2-theta peaks in Table 13 having a relative intensity of greater than 10%.

114. The crystalline form of embodiment 111, having an X-ray powder diffraction (XRPD) pattern according to FIG. 13A.

115. The crystalline form of embodiment 111, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 13.9 | 6.4 |
| 15.2 | 5.8 |
| 16.4 | 5.4 |
| 18.1 | 4.9 |
| 19.5 | 4.6. |

116. The crystalline form of embodiment 111, characterized by an X-ray powder diffraction (XRPD) comprising the following 2-theta peaks (2-theta±0.2) and corresponding d-spacing

| 2θ[°] | d-spacing [Å] |
|---|---|
| 11.8 | 7.5 |
| 12.4 | 7.1 |
| 13.9 | 6.4 |
| 14.5 | 6.1 |
| 15.2 | 5.8 |
| 16.4 | 5.4 |
| 18.1 | 4.9 |
| 19.5 | 4.6 |
| 20.1 | 4.4 |
| 20.7 | 4.3 |
| 22.0 | 4.0 |
| 23.5 | 3.8 |
| 23.9 | 3.7 |
| 25.2 | 3.5 |
| 25.6 | 3.5. |

117. The crystalline form of any one of embodiments 111-116, characterized by a TGA thermogram according to FIG. 13B.

118. The crystalline form of any one of embodiments 111-116, further characterized by a sample weight loss of up to about 11% upon heating to 180° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

119. The crystalline form of any one of embodiments 111-118, characterized by a differential scanning calorimetry (DSC) thermogram according to FIG. 13B.

120. The crystalline form of any one of embodiments 111-118, further characterized by a DSC having an endotherm at about 193° C. to about 198° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

121. Crystalline Oxalate Form A of mesembranol having an X-ray powder diffraction (XRPD) pattern comprising 2-theta peaks (2-theta±0.2) at 12.0°, 15.9°, 18.3°, 19.4°, and 20.0°.

122. The crystalline form of embodiment 121, having an X-ray powder diffraction (XRPD) pattern comprising 2-theta peaks (2-theta±0.2) at 12.0°, 12.5°, 15.9°, 17.1°, 18.3°, 19.4°, 20.0°, 22.0°, 24.0°, 24.2°, 25.7°, 26.6°, 29.2°, and 31.8°.

123. The crystalline form of embodiment 121, having an X-ray powder diffraction (XRPD) pattern comprising the 2-theta peaks in Table 14 having a relative intensity of greater than 10%.

124. The crystalline form of embodiment 121, having an X-ray powder diffraction (XRPD) pattern according to FIG. 14A.

125. The crystalline form of embodiment 121, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 12.0 | 7.4 |
| 15.9 | 5.6 |
| 18.3 | 4.8 |
| 19.4 | 4.6 |
| 20.0 | 4.4. |

126. The crystalline form of embodiment 121, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 12.0 | 7.4 |
| 12.5 | 7.1 |
| 15.9 | 5.6 |
| 17.1 | 5.2 |
| 18.3 | 4.8 |
| 19.4 | 4.6 |
| 20.0 | 4.4 |
| 22.0 | 4.0 |
| 24.0 | 3.7 |
| 24.2 | 3.7 |
| 25.7 | 3.5 |
| 26.6 | 3.4 |
| 29.2 | 3.1 |
| 31.8 | 2.8. |

127. The crystalline form of any one of embodiments 121-126, characterized by a TGA thermogram according to FIG. 14B.

128. The crystalline form of any one of embodiments 121-126, further characterized by a sample weight loss of up to about 9% upon heating to 100° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

129. The crystalline form of any one of embodiments 121-128, characterized by a differential scanning calorimetry (DSC) thermogram according to FIG. 14B.

130. The crystalline form of any one of embodiments 121-128, further characterized by a DSC having an endotherm at about 95° C. to about 100° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

131. Crystalline HBr Form A of mesembranol having an X-ray powder diffraction (XRPD) pattern comprising 2-theta peaks (2-theta±0.2) at 16.4°, 17.2°, 18.6°, 23.4°, and 26.2°.

132. The crystalline form of embodiment 131, having an X-ray powder diffraction (XRPD) pattern comprising 2-theta peaks (2-theta±0.2) at 11.6°, 16.4°, 17.2°, 17.9°, 18.6°, 20.9°, 21.4°, 23.0°, 23.4°, 24.9°, 25.9°, 26.2°, 27.6°, and 30.3°.

133. The crystalline form of embodiment 131, having an X-ray powder diffraction (XRPD) pattern comprising the 2-theta peaks in Table 15 having a relative intensity of greater than 10%. 134. The crystalline form of embodiment 131, having an X-ray powder diffraction (XRPD) pattern according to FIG. 15A.

135. The crystalline form of embodiment 131, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 16.4 | 5.4 |
| 17.2 | 5.1 |
| 18.6 | 4.8 |
| 23.4 | 3.8 |
| 26.2 | 3.4. |

136. The crystalline form of embodiment 131, characterized by an X-ray powder diffraction (XRPD) comprising the following 2-theta peaks (2-theta±0.2) and corresponding d-spacing (angstroms±0.2):

| 2θ[°] | d-spacing [Å] |
|---|---|
| 11.6 | 7.6 |
| 16.4 | 5.4 |
| 17.2 | 5.1 |
| 17.9 | 5.0 |
| 18.6 | 4.8 |
| 20.9 | 4.3 |
| 21.4 | 4.2 |
| 23.0 | 3.9 |
| 23.4 | 3.8 |
| 24.9 | 3.6 |
| 25.9 | 3.4 |
| 26.2 | 3.4 |
| 27.6 | 3.2 |
| 30.3 | 3.0. |

137. The crystalline form of any one of embodiments 131-136, characterized by a TGA thermogram according to FIG. 15B.

138. The crystalline form of any one of embodiments 131-136, further characterized by a sample weight loss of up to about 5% upon heating to 80° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂. |

139. The crystalline form of any one of embodiments 131-138, characterized by a differential scanning calorimetry (DSC) thermogram according to FIG. 15B.

140. The crystalline form of any one of embodiments 131-138, further characterized by a DSC having an endotherm at about 200° C. to about 205° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

141. A pharmaceutical composition, comprising a solid form of any one of embodiments 1-140; and a pharmaceutically acceptable excipient.

142. A process for preparing a pharmaceutical composition, comprising dissolving in a solvent a solid form any one of embodiments 1-140.

143. A method of treating a mental health disorder, comprising administering to a mammal in need thereof an effective amount of a solid form according to any one of embodiments 1-140 or a pharmaceutical composition of embodiment 141.

144. The method of embodiment 143, wherein the mental health disorder is anxiety, stress, or depression.

145. The method of embodiment 143, wherein the mental health disorder is anxiety.

146. The method of embodiment 143, wherein the mental health disorder is stress.

147. The method of embodiment 143, wherein the mental health disorder is depression.

148. The method of any one of embodiments 143-147, wherein the mammal is a human.

149. A composition comprising a solid form of mesembranol in combination with one or more compounds selected from the group consisting of tartaric acid, fumaric acid, malic acid, maleic acid, naphthalene-1,5-disulfonic acid, hydrobromic acid, hydrochloric acid, oxalic acid, phosphoric acid, sulfuric acid, and gentisic acid.

150. The composition of embodiment 149, wherein the mesembranol is (−) mesembranol.

151. A method of treating a mental health disorder, comprising administering to a mammal in need thereof an effective amount of a solid form of (−) mesembranol selected from the group consisting of:
a.) Sulfate Form A having an X-ray powder diffraction (XRPD) pattern comprising a 2-theta peak (2-theta±0.2) at 16.1°;
b.) Malate Form A having an X-ray powder diffraction (XRPD) pattern comprising a 2-theta peak (2-theta±0.2) at 13.1°;
c.) Malate Form B having an X-ray powder diffraction (XRPD) pattern comprising a 2-theta peak (2-theta±0.2) at 18.1;
d.) Gentisate Form A having an X-ray powder diffraction (XRPD) pattern comprising a 2-theta peak (2-theta±0.2) at 16.6°;
e.) Maleate Form A having an X-ray powder diffraction (XRPD) pattern comprising a 2-theta peak (2-theta±0.2) at 14.2°;
f.) HCl Form A having an X-ray powder diffraction (XRPD) pattern comprising a 2-theta peak (2-theta±0.2) at 16.6°;
g.) HCl Form B having an X-ray powder diffraction (XRPD) pattern comprising a 2-theta peak (2-theta±0.2) at 16.8°;
h.) HCl Form C having an X-ray powder diffraction (XRPD) pattern comprising a 2-theta peak (2-theta±0.2) at 17.9°;
i.) Phosphate Form A having an X-ray powder diffraction (XRPD) pattern comprising a 2-theta peak (2-theta±0.2) at 3.9°;
j.) Tartrate Form A having an X-ray powder diffraction (XRPD) pattern comprising a 2-theta peak (2-theta±0.2) at 5.0°;
k.) Fumarate Form A having an X-ray powder diffraction (XRPD) pattern comprising a 2-theta peak (2-theta±0.2) at 12.7°;
l.) Napthalene-1,5-disulfonate Form A having an X-ray powder diffraction (XRPD) pattern comprising a 2-theta peak (2-theta±0.2) at 13.9°;
m.) Oxalate Form A having an X-ray powder diffraction (XRPD) pattern comprising a 2-theta peak (2-theta±0.2) at 18.3°; and
n.) HBr Form A having an X-ray powder diffraction (XRPD) pattern comprising a 2-theta peak (2-theta±0.2) at 16.4°.

152. The method of embodiment 151, wherein the recited XRPD peak for each solid form is the most intense peak by relative intensity in the respective XRPD patterns.

153. A method of treating a mental health disorder, comprising administering to a mammal in need thereof an effective amount of a solid form of (−) mesembranol selected from the group consisting of:
a.) Sulfate Form A having an X-ray powder diffraction (XRPD) pattern comprising a 2-theta peak (2-theta±0.2) at 16.1°;
b.) Malate Form A having an X-ray powder diffraction (XRPD) pattern comprising a 2-theta peak (2-theta±0.2) at 13.1°;
c.) Malate Form B having an X-ray powder diffraction (XRPD) pattern comprising a 2-theta peak (2-theta±0.2) at 20.2;
d.) Gentisate Form A having an X-ray powder diffraction (XRPD) pattern comprising a 2-theta peak (2-theta±0.2) at 16.6°;
e.) Maleate Form A having an X-ray powder diffraction (XRPD) pattern comprising a 2-theta peak (2-theta±0.2) at 14.2°;
f.) HCl Form A having an X-ray powder diffraction (XRPD) pattern comprising a 2-theta peak (2-theta±0.2) at 18.5°;
g.) HCl Form B having an X-ray powder diffraction (XRPD) pattern comprising a 2-theta peak (2-theta±0.2) at 19.8°;
h.) HCl Form C having an X-ray powder diffraction (XRPD) pattern comprising a 2-theta peak (2-theta±0.2) at 9.0°;
i.) Phosphate Form A having an X-ray powder diffraction (XRPD) pattern comprising a 2-theta peak (2-theta±0.2) at 3.9°;
j.) Tartrate Form A having an X-ray powder diffraction (XRPD) pattern comprising a 2-theta peak (2-theta±0.2) at 5.0°;
k.) Fumarate Form A having an X-ray powder diffraction (XRPD) pattern comprising a 2-theta peak (2-theta±0.2) at 12.7°;
l.) Napthalene-1,5-disulfonate Form A having an X-ray powder diffraction (XRPD) pattern comprising a 2-theta peak (2-theta±0.2) at 18.1°;
m.) Oxalate Form A having an X-ray powder diffraction (XRPD) pattern comprising a 2-theta peak (2-theta±0.2) at 15.9°; and
n.) HBr Form A having an X-ray powder diffraction (XRPD) pattern comprising a 2-theta peak (2-theta±0.2) at 16.4°.

### Examples

### Instruments and Methods

### XRPD

For XRPD analysis, Empyrean and X′pert³ X-ray powder diffractometers from Panalytical were used. Sample was spread on the middle of a zero-background Si holder. The XRPD parameters used are listed in Table 1A.

TABLE 1A
Parameters for XRPD test

| Model | Empyrean | X'Pert³ |
|---|---|---|
| X-Ray wavelength | Cu, kα, Kα1 (Å): 1.540598, Kα2 (Å): 1.544426 | |
| | Kα2/Kα1 intensity ratio: 0.50 | |
| X-Ray tube setting | 45 kV, 40 mA | 45 kV, 40 mA |
| Divergence slit | ⅛° | ⅛° |
| Scan mode | Continuous | Continuous |
| Scan range (°2TH) | 3~40 | 3~40 |
| Scan step time (s) | 46.667 | 46.667 |
| Step size (°2TH) | 0.0263 | 0.0263 |
| Test time | 5 min 4 s | 5 min 4 s |

### TGA

TGA data were collected using a TA Discovery TGA 5500 TGA from TA Instruments. Detailed parameters used are listed in Table 1B.

TABLE 1B
Parameters for TGA test

| Parameters | TGA | |
|---|---|---|
| Method | Ramp | |
| Sample pan | Aluminum, open | |
| Temperature | RT-350 | °C |
| Heating rate | 10 | °C/min |

| | |
|---|---|
| Purge gas | N₂ |

### DSC

DSC data were collected using a TA Discovery DSC 2500 DSC from TA Instruments. Detailed parameters used are listed in Table 1C.

TABLE 1C
Parameters for DSC test

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂ |

### HPLC Conditions

Waters H-Class UPLC was utilized for sample analysis and detailed chromatographic conditions are listed in Table 1D.

| Parameters | Waters H-class | |
|---|---|---|
| Column | ACQUITY UPLC BEH C18, 50 × 2.1 mm, 1.7 μm | |
| Mobile phase | A: 0.1% TFA in H₂O | |
| | B: 0.1% TFA in ACN | |
| | Time (min) | % B |
| Gradient table | 0.0 | 5 |
| | 10.0 | 30 |
| | 12.0 | 95 |
| | 12.1 | 5 |
| | 15.0 | 5 |

| | |
|---|---|
| Run time | 15.0 min |
| Post time | 0.0 min |
| Flow rate | 0.5 mL/min |
| Injection volume | 1.6 μL |
| Detector wavelength | 230 nm |
| Column temperature | 40 °C |
| Sampler temperature | RT |
| Diluent | ACN/H₂O (1:1, v/v) |

### Ion Chromatogram

ThermoFisher ICS-1100 was utilized and detailed conditions are listed in Table 1E.
Table 1E. Ion chromatograph conditions and parameteres

| | |
|---|---|
| IC | ThermoFisher ICS-1100 |
| Column | Dionex IonPac AS18 RFIC (4 × 250 mm) |
| Mobile phase | 25 mM NaOH |
| Injection volume | 25 μL |
| Flow rate | 1.0 mL/min |
| Cell temperature | 35 °C |
| IC | ThermoFisher ICS-1100 |
| Column temperature | 35 °C |
| Current | 80 mA |
| Run time | 25 min for Cl⁻, SO4²⁻, PO4³⁻, C₂O4²⁻ and Br⁻ |

### Synthesis of (+/−)-Mesembrine

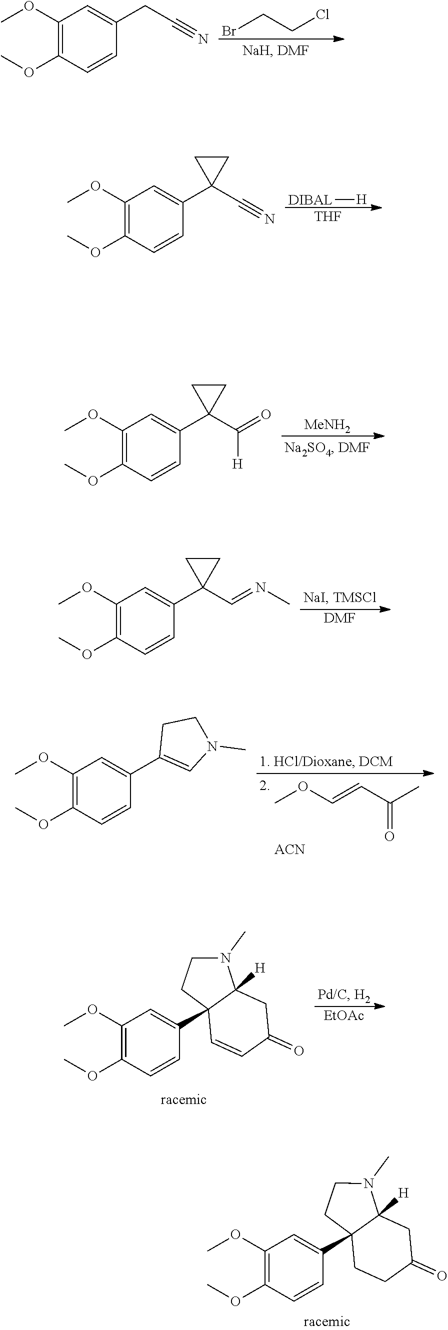

### Step 1: Synthesis of 1-(3,4-dimethoxyphenyl)cyclopropane-1-carbonitrile

To a solution of 2-(3,4-dimethoxyphenyl) acetonitrile (20 g, 112 mmol) in DMF (93 mL) was added NaH (18.0 g, 451 mmol, 60% purity) in portions. The mixture was allowed to stir at 25° C. for 20 min. 1-Bromo-2-chloro-ethane (16.1 g, 112 mmol) was added, and the mixture was allowed to stir at 25° C. for 16 hr. The reaction was quenched by the addition of a MeOH/water mixture (1:1; 1000 mL) and the resulting solution was extracted with EtOAc (3×500 mL). The organic solutions were combined, washed with water (4×500 mL) and brine (1×200 mL) and dried over (Na₂SO₄). The solution was filtered and the solvent was evaporated under reduced pressure. The resulting solid was purified by column chromatography (SiO₂, Petroleum ether/EtOAc=10/1 to 3/1) to give 1-(3,4-dimethoxyphenyl)cyclopropane-1-carbonitrile (15 g, 65%) as yellow oil. ¹H NMR (400 MHz, CDCl₃) δ 6.88 (s, 1H), 6.82 (d, J=1.2 Hz, 2H), 3.91 (s, 3H), 3.88 (s, 3H), 1.68-1.65 (m, 2H), 1.35 (d, J=2.4 Hz, 2H).

### Step 2: Synthesis of 1-(3,4-dimethoxyphenyl)cyclopropane-1-carbaldehyde

To a solution of 1-(3,4-dimethoxyphenyl)cyclopropane-1-carbonitrile (11 g, 54.1 mmol) in THF (160 mL) was added DIBAL-H (1M in toluene, 81.2 mL). The mixture was allowed to stir at 25° C. for 3 hr and then the reaction was cautiously quenched by addition of aqueous 2M HCl. The solution was extracted with DCM (3×200 mL). The organic solutions were combined, washed with water (2×200 mL) and brine (2×200 mL), and then dried over Na₂SO₄ to give 1-(3,4-dimethoxyphenyl)cyclopropane-1-carbaldehyde (9.6 g, 85%) as yellow oil. LC-MS (ESI+) m/z 207.0 (M+H)⁺. ¹H NMR (400 MHz, CDCl₃) δ 9.26 (s, 1H), 6.94-6.61 (m, 3H), 3.89 (d, J=2.8 Hz, 6H), 1.61-1.52 (m, 2H), 1.42-1.37 (m, 2H)

### Step 3: Synthesis of (Z)-1-(1-(3,4-dimethoxyphenyl)cyclopropyl)-N-methylmethanimine

To a solution of 1-(3,4-dimethoxyphenyl)-cyclopropanecarbaldehyde (5.0 g, 24.2 mmol) in DCM (50 mL) was added MeNH₂ (2 M, 121 mL) and Na₂SO₄ (15.5 g, 109 mmol, 11.0 mL). The mixture was allowed to stir at 25° C. for 16 hr. The reaction mixture was filtered and the filtrate was concentrated under reduced pressure to give (Z)-1-(1-(3,4-dimethoxyphenyl)cyclopropyl)-N-methylmethanimine (5.1 g, 99%) as white solid. LC-MS (ESI+) m/z 219.9 (M+H)⁺; ¹H NMR (400 MHz, CDCl₃) δ 7.55 (q, J=1.2 Hz, 1H), 6.93-6.77 (m, 3H), 3.88 (d, J=7.2 Hz, 6H), 3.24 (d, J=1.6 Hz, 3H), 1.29-1.23 (m, 2H), 1.18-1.12 (m, 2H).

### Step 4: Synthesis of 4-(3,4-dimethoxyphenyl)-1-methyl-2,3-dihydro-1H-pyrrole

To a solution of (Z)-1-(1-(3,4-dimethoxyphenyl)cyclopropyl)-N-methylmethanimine (5.4 g, 24.6 mmol) in DMF (19 mL) was added NaI (366 mg, 2.44 mmol) and TMSCl (267 mg, 2.46 mmol). The mixture was allowed to stir at 90° C. for 3 hr. The reaction mixture was diluted with water (100 mL) and extracted with EtOAc (100 mL×3). The organic solutions were combined, washed with water and brine, dried over Na₂SO₄, and filtered. The filtrate was concentrated under reduced pressure to give 4-(3,4-dimethoxyphenyl)-1-methyl-2,3-dihydro-1H-pyrrole (6.25 g, 80%) as yellow oil. LC-MS (ESI+) m/z 220.0 (M+H)⁺. ¹H NMR (400 MHz, CDCl₃) δ 6.90-6.66 (m, 3H), 6.31 (t, J=1.6 Hz, 1H), 3.95-3.80 (m, 6H), 3.18-3.11 (m, 2H), 2.79 (dt, J=1.2, 9.0 Hz, 2H), 2.65 (s, 3H).

### Step 5: Synthesis of rac-3a-(3,4-dimethoxyphenyl)-1-methyl-1,2,3,3a,7,7a-hexahydro-6H-indol-6-one

4-(3,4-Dimethoxyphenyl)-1-methyl-2,3-dihydro-1H-pyrrole (6.25 g, 28.5 mmol) was dissolved in DCM (100 mL). To this solution was added HCl (1M in dioxane, 25 mL, 100 mmol). The mixture was evaporated to dryness and then dissolved in ACN (90 mL). To this solution was added (E)-4-methoxybut-3-en-2-one (4.28 g, 42.7 mmol). The reaction mixture was allowed to stir at 90° C. for 16 hr. The reaction mixture was filtered and the filtrate was concentrated under reduced pressure. The residue was purified by HPLC (column: Phenomenex luna C18 (250*70 mm, 10 μm); mobile phase: [water (NH₄HCO₃)-ACN]; B %: 22%-52%, 20 min). The eluent was acidified with aq. HCl to give rac-3a-(3,4-dimethoxyphenyl)-1-methyl-1,2,3,3a, 7,7a-hexahydro-6H-indol-6-one (3.0 g, 30%) as a white solid. LC-MS (ESI*) m/z 288.3 (M+H)⁺. 1H NMR (400 MHz, CDCl₃) δ 6.90-6.88 (m, 1H), 6.87-6.83 (m, 2H), 6.74 (dd, J=2.0, 10.1 Hz, 1H), 6.11 (d, J=10.0 Hz, 1H), 3.89 (d, J=4.0 Hz, 6H), 3.33 (dt, J=2.4, 8.8 Hz, 1H), 2.69-2.66 (m, 1H), 2.58-2.51 (m, 2H), 2.50-2.41 (m, 2H), 2.33 (s, 3H), 2.27-2.18 (m, 1H)

### Step 6: Synthesis of rac-3a-(3,4-dimethoxyphenyl)-1-methyloctahydro-6H-indol-6-one

A mixture of rac-3a-(3,4-dimethoxyphenyl)-1-methyl-1,2,3,3a,7,7a-hexahydro-6H-indol-6-one (12.0 g, 43.9 mmol) and 10% Pd/C (300 mg) in EtOAc (120 mL) was degassed and then purged with H₂ for 3 times. The mixture was allowed to stir at 25° C. for 2 hr under 15 psi H₂. The reaction mixture was filtered and the filtrate was concentrated in vacuo to give rac-3a-(3,4-dimethoxyphenyl)-1-methyloctahydro-6H-indol-6-one (10 g, 80%) as brown oil. LC-MS (ESI+) m/z 290.4 (M+H)⁺. ¹H NMR (400 MHz, CDCl₃) δ 6.99-6.89 (m, 2H), 6.89-6.84 (m, 1H), 3.91 (d, J=7.6 Hz, 6H), 3.20-3.11 (m, 1H), 2.97 (t, J=3.6 Hz, 1H), 2.69-2.56 (m, 2H), 2.51-2.31 (m, 5H), 2.27-2.18 (m, 3H), 2.18-2.07 (m, 2H).

### Chiral resolution of rac-3a-(3,4-dimethoxyphenyl)-1-methyloctahydro-6H-indol-6-one to give (−) mesembrine

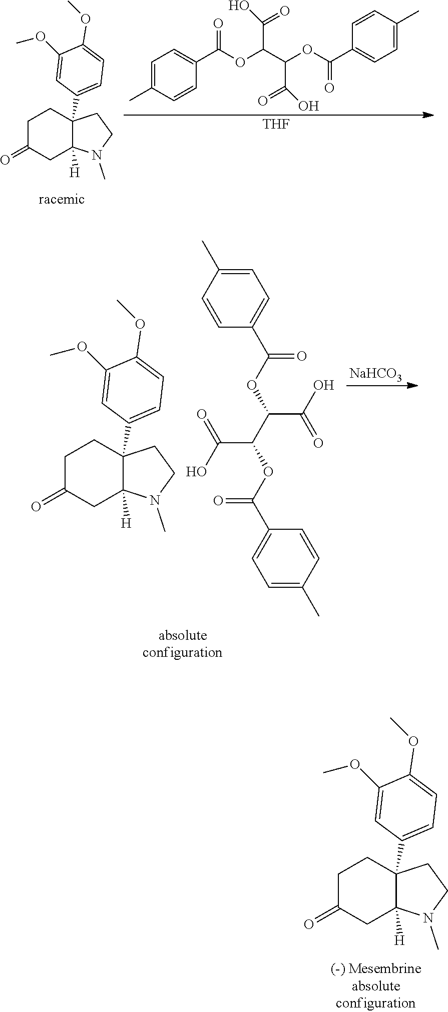

To a solution of rac-3a-(3,4-dimethoxyphenyl)-1-methyloctahydro-6H-indol-6-one (28.0 g, 85.1 mmol) in THF (1400 mL) was added (2S,3S)-2,3-bis[(4-methylbenzoyl)oxy]butanedioic acid (19.7 g, 51.1 mmol). The suspension was allowed to stir at 25° C. for 16 hr and then filtered. The solid was dried in vacuo and then triturated 3 times with THF at 25° C. for 16 hr. The resulting solid was filtered and then added to a saturated sodium bicarbonate solution (500 mL). The mixture was extracted with EtOAc (500 mL). The organic solutions were combined, dried over Na₂SO₄, and filtered. The filtrate was concentrated in vacuo to give (−)-Mesembrine (7.50 g, 95% ee) as a yellow gum. LC-MS (ESI+) m/z 290.6 (M+H)⁺. ¹H NMR (400 MHz, CDCl₃) δ 6.89-6.81 (m, 2H), 6.80-6.75 (m, 1H), 3.82 (d, J=8.0 Hz, 6H), 3.11-3.03 (m, 1H), 2.88 (t, J=3.6 Hz, 1H), 2.59-2.48 (m, 2H), 2.43-2.32 (m, 1H), 2.31-2.21 (m, 4H), 2.20-2.09 (m, 3H), 2.08 (br s, 2H).

### Synthesis of (3aS,6R,7aS)-3a-(3,4-dimethoxyphenyl)-1-methyloctahydro-1H-indol-6-ol ((−)-mesembranol (Compound 1))

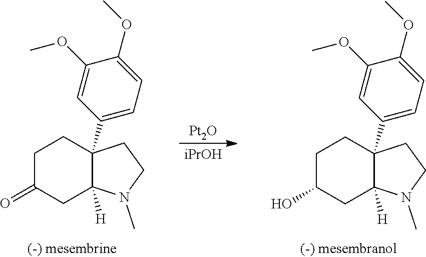

A mixture of mesembrine (200 mg, 691 μmol) and PtO₂ (20.0 mg, 88.0 μmol) in IPA (4 mL) was degassed and purged with N₂ 3 times. The mixture was allowed to stir at 25° C. for 16 hr under an atmosphere of N₂. The reaction mixture was filtered and concentrated under reduced pressure. The residue was purified by re-crystallization from EtOH (1 mL) at 25° C. to give (3aS,6R,7aS)-3a-(3,4-dimethoxyphenyl)-1-methyloctahydro-1H-indol-6-ol ((−)-mesembranol) (100 mg, 49%) as a white solid. LC-MS (ESI+) m/z 292.4 (M+H). ¹H NMR (400 MHz, CDCl₃) δ 6.86-6.78 (m, 2H), 6.77-6.71 (m, 1H), 3.86 (s, 1H), 3.81 (d, J=6.8 Hz, 6H), 3.30 (dt, J=6.8, 9.6 Hz, 1H), 2.83 (s, 1H), 2.40 (s, 3H), 2.33-2.20 (m, 1H), 2.09 (dd, J=2.8, 14.8 Hz, 1H), 1.90-1.82 (m, 2H), 1.78 (dd, J=6.8, 11.6 Hz, 1H), 1.67-1.62 (m, 2H), 1.57 (td, J=2.8, 14.8 Hz, 1H), 1.39-1.30 (m, 2H).

### Characterization of (−) Mesembranol

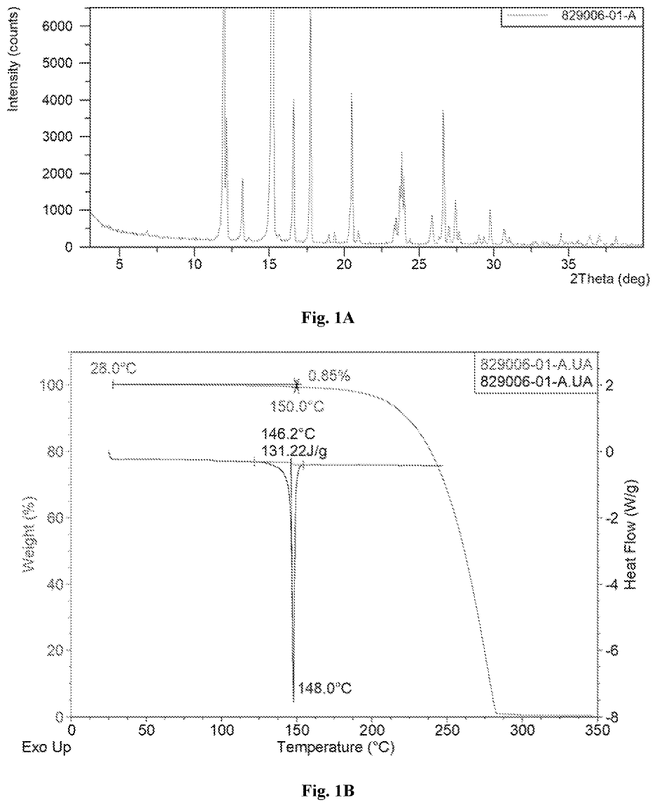

(−) Mesembranol was characterized by XRPD, TGA, DSC, ¹H NMR and UPLC. The XRPD result is shown in FIG. 1A, and showed the starting material was crystalline, named as Freeform Form A. TGA/DSC curves are shown in FIG. 1B. The TGA result showed a weight loss of 0.85% up to 150° C. and DSC result showed an endotherm at 146.2° C. (onset temperature). Due to the limited TGA weight loss and neat DSC signal before the possible melting, Freeform Form A was speculated to be an anhydrate. ¹H NMR data was collected using DMSO-d₆ as solvent. The purity of Freeform Form A was determined to be 98.73 area % by UPLC.

### Example 1—Sulfate Form A

Sulfate Form A was obtained via slurry of Freeform Form A and equal molar H₂SO₄ in acetone at RT for 3 days and drying at RT under vacuum for 8 hrs. XRPD result is shown in FIG. 2A. TGA/DSC curves shown in FIG. 2B show a weight loss of 3.85% up to 200° C. and an endotherm at 160.4° C. (peak temperature). ¹H NMR spectrum showed no obvious residual solvent acetone was detected. The stoichiometric ratio of SO₄²⁻/freeform was 1.0 by UPLC/IC. The purity of Sulfate Form A was determined to be 99.83 area % by UPLC.

TABLE 2

| #Peak | 2θ[°] | d-spacing [Å] | Rel. Int. [%] |
|---|---|---|---|
| 1 | 5.85 | 15.10 | 7.74 |
| 2 | 11.17 | 7.92 | 14.26 |
| 3 | 11.68 | 7.58 | 12.72 |
| 4 | 11.96 | 7.40 | 3.87 |
| 5 | 12.26 | 7.22 | 4.01 |
| 6 | 12.98 | 6.82 | 17.41 |
| 7 | 13.89 | 6.38 | 23.16 |
| 8 | 14.53 | 6.10 | 12.34 |
| 9 | 15.83 | 5.60 | 14.91 |
| 10 | 16.10 | 5.50 | 100.00 |
| 11 | 16.46 | 5.38 | 29.77 |
| 12 | 16.88 | 5.25 | 27.51 |
| 13 | 17.53 | 5.06 | 56.52 |
| 14 | 18.11 | 4.90 | 28.96 |
| 15 | 18.67 | 4.75 | 29.66 |
| 16 | 19.71 | 4.51 | 2.19 |
| 17 | 20.61 | 4.31 | 11.81 |
| 18 | 21.06 | 4.22 | 2.58 |
| 19 | 21.58 | 4.12 | 9.37 |
| 20 | 21.80 | 4.08 | 10.40 |
| 21 | 22.41 | 3.97 | 1.97 |
| 22 | 23.17 | 3.84 | 8.32 |
| 23 | 23.43 | 3.80 | 34.30 |
| 24 | 23.70 | 3.75 | 3.93 |
| 25 | 23.99 | 3.71 | 4.30 |
| 26 | 24.56 | 3.63 | 11.33 |
| 27 | 24.74 | 3.60 | 25.95 |
| 28 | 25.26 | 3.53 | 12.21 |
| 29 | 25.83 | 3.45 | 17.84 |
| 30 | 25.98 | 3.43 | 18.20 |
| 31 | 26.34 | 3.38 | 14.03 |
| 32 | 27.15 | 3.28 | 9.31 |
| 33 | 27.54 | 3.24 | 6.15 |
| 34 | 27.94 | 3.19 | 1.51 |
| 35 | 28.26 | 3.16 | 7.05 |
| 36 | 29.38 | 3.04 | 6.78 |
| 37 | 29.63 | 3.01 | 7.84 |
| 38 | 30.31 | 2.95 | 2.38 |
| 39 | 30.91 | 2.89 | 1.81 |
| 40 | 31.23 | 2.86 | 2.48 |
| 41 | 31.66 | 2.83 | 1.88 |
| 42 | 32.04 | 2.79 | 3.09 |
| 43 | 33.12 | 2.70 | 1.39 |
| 44 | 33.55 | 2.67 | 1.21 |
| 45 | 34.13 | 2.63 | 1.55 |
| 46 | 34.82 | 2.58 | 2.01 |
| 47 | 35.42 | 2.53 | 2.93 |
| 48 | 35.85 | 2.50 | 2.49 |
| 49 | 36.27 | 2.48 | 1.78 |
| 50 | 37.12 | 2.42 | 2.71 |

### Example 2—Malate Form A

Malate Form A was obtained via slurry of Freeform Form A and equal molar L-malic acid in acetone at RT for 3 days and drying at RT under vacuum for 8 hrs. TGA result showed a weight loss of 2.31% up to 150° C. DSC result showed two endotherms at 90.1° C. and 142.8° C. (peak temperature). ¹H NMR result showed that the molar ratio of acid/freeform was 1.0 and residual solvent acetone/API was 0.02 (0.3 wt %). The purity was determined to be 99.78 area %. After heating Malate Form A to 100° C., cooling to RT and exposure to ambient conditions, XRPD results (FIG. 3A) showed that several weak peaks disappeared. The disappeared peaks might be attributed to Malate Form B, indicating the sample might be a mixture of Malate Type A+B and the heated sample was pure Malate Form A. TGA/DSC results of Malate Form A in FIG. 3B showed a weight loss of 1.86% up to 150° C. and one endotherm at 143.4° C. (peak temperature). ¹H NMR result showed that the molar ratio of acid/freeform was 1.0 and negligible solvent acetone was detected.

TABLE 3

| #Peak | Pos. [°2θ] | d-spacing [Å] | Rel. Int. [%] |
|---|---|---|---|
| 1 | 11.32 | 7.82 | 15.83 |
| 2 | 12.66 | 6.99 | 47.00 |
| 3 | 13.10 | 6.76 | 100.00 |
| 4 | 16.02 | 5.53 | 90.21 |
| 5 | 16.36 | 5.42 | 14.90 |
| 6 | 19.95 | 4.45 | 36.17 |
| 7 | 20.44 | 4.34 | 8.04 |
| 8 | 20.75 | 4.28 | 61.21 |
| 9 | 21.53 | 4.13 | 81.20 |
| 10 | 22.71 | 3.92 | 13.11 |
| 11 | 22.95 | 3.88 | 11.09 |
| 12 | 23.41 | 3.80 | 13.29 |
| 13 | 23.68 | 3.76 | 75.03 |
| 14 | 24.39 | 3.65 | 38.96 |
| 15 | 25.42 | 3.50 | 5.22 |
| 16 | 25.64 | 3.47 | 11.39 |
| 17 | 26.28 | 3.39 | 9.31 |
| 18 | 26.91 | 3.31 | 6.50 |
| 19 | 28.47 | 3.13 | 12.50 |
| 20 | 29.30 | 3.05 | 5.34 |
| 21 | 30.36 | 2.94 | 2.76 |
| 22 | 30.97 | 2.89 | 10.78 |
| 23 | 31.42 | 2.85 | 5.06 |
| 24 | 31.84 | 2.81 | 2.87 |
| 25 | 32.42 | 2.76 | 1.62 |
| 26 | 33.06 | 2.71 | 8.12 |
| 27 | 33.88 | 2.65 | 5.19 |
| 28 | 36.37 | 2.47 | 8.91 |

### Example 3—Malate Form B

Malate Form B was obtained via slurry of Freeform Form A and equal molar L-malic acid in 2-MeTHF at RT for 3 days and drying at RT under vacuum for 8 hrs. The XRPD pattern is shown in FIG. 4A TGA/DSC curves are shown in FIG. 3B. TGA result showed a weight loss of 4.38% up to 110° C. DSC result showed an endotherm at 101.7° C. (peak temperature). ¹H NMR results show that the molar ratio of acid/freeform was 1.1 and residual solvent 2-MeHTF/API was 0.03 (0.5 wt %). The purity of Malate Form B was determined to be 98.86 area % by UPLC.

TABLE 4

| #Peak | Pos. [°2θ] | d-spacing [Å] | Rel. Int. [%] |
|---|---|---|---|
| 1 | 4.55 | 19.42 | 46.95 |
| 2 | 9.05 | 9.77 | 10.48 |
| 3 | 12.52 | 7.07 | 32.95 |
| 4 | 12.76 | 6.94 | 61.22 |
| 5 | 13.53 | 6.54 | 56.20 |
| 6 | 16.45 | 5.39 | 23.35 |
| 7 | 16.86 | 5.26 | 42.39 |
| 8 | 17.30 | 5.12 | 6.70 |
| 9 | 18.10 | 4.90 | 100.00 |
| 10 | 19.02 | 4.67 | 24.46 |
| 11 | 19.71 | 4.50 | 30.89 |
| 12 | 19.88 | 4.47 | 43.57 |
| 13 | 20.21 | 4.39 | 18.51 |
| 14 | 21.58 | 4.12 | 81.05 |
| 15 | 23.57 | 3.77 | 9.36 |
| 16 | 23.89 | 3.73 | 5.98 |
| 17 | 24.37 | 3.65 | 5.03 |
| 18 | 24.73 | 3.60 | 12.08 |
| 19 | 25.65 | 3.47 | 8.04 |
| 20 | 26.47 | 3.37 | 33.05 |
| 21 | 27.10 | 3.29 | 6.98 |
| 22 | 27.78 | 3.21 | 27.90 |
| 23 | 28.29 | 3.15 | 8.38 |
| 24 | 29.09 | 3.07 | 2.69 |
| 25 | 30.21 | 2.96 | 10.56 |
| 26 | 31.98 | 2.80 | 7.02 |
| 27 | 33.83 | 2.65 | 6.21 |
| 28 | 36.66 | 2.45 | 4.42 |
| 29 | 38.19 | 2.36 | 3.40 |
| 30 | 38.72 | 2.33 | 4.75 |

### Example 4—Gentisate Form A

Gentisate Form A was obtained via slurry of Freeform Form A and equal molar gentisic acid in 2-MeTHF at RT for 3 days and drying at RT under vacuum for 8 hrs. XRPD overlay was displayed in FIG. 5A. TGA/DSC curves, shown in FIG. 5B, showed a weight loss of 2.10% up to 150° C. and an endotherm at 194.4° C. (peak temperature). ¹H NMR spectrum showed the molar ratio of acid/freeform was 1.0 and residual solvent 2-MeTHF/API was 0.04 (0.8 wt %). The purity of Gentisate Form A was determined to be 99.10 area % by UPLC.

TABLE 5

| #Peak | Pos. [°2θ] | d-spacing [Å] | Rel. Int. [%] |
|---|---|---|---|
| 1 | 8.96 | 9.87 | 28.40 |
| 2 | 10.71 | 8.26 | 2.56 |
| 3 | 11.46 | 7.72 | 22.19 |
| 4 | 12.91 | 6.86 | 18.37 |
| 5 | 13.17 | 6.73 | 5.56 |
| 6 | 13.65 | 6.49 | 23.01 |
| 7 | 14.15 | 6.26 | 16.46 |
| 8 | 15.97 | 5.55 | 6.44 |
| 9 | 16.56 | 5.35 | 100.00 |
| 10 | 17.13 | 5.18 | 31.82 |
| 11 | 17.92 | 4.95 | 10.14 |
| 12 | 18.99 | 4.67 | 30.06 |
| 13 | 19.37 | 4.58 | 10.31 |
| 14 | 20.25 | 4.39 | 7.38 |
| 15 | 20.52 | 4.33 | 11.29 |
| 16 | 21.02 | 4.23 | 10.63 |
| 17 | 21.50 | 4.13 | 16.10 |
| 18 | 21.93 | 4.05 | 16.02 |
| 19 | 22.21 | 4.00 | 14.66 |
| 20 | 22.99 | 3.87 | 9.31 |
| 21 | 23.45 | 3.79 | 9.26 |
| 22 | 23.72 | 3.75 | 13.85 |
| 23 | 24.24 | 3.67 | 28.10 |
| 24 | 25.15 | 3.54 | 12.01 |
| 25 | 26.46 | 3.37 | 8.49 |
| 26 | 26.99 | 3.30 | 4.25 |
| 27 | 27.45 | 3.25 | 9.00 |
| 28 | 29.52 | 3.03 | 2.85 |
| 29 | 30.62 | 2.92 | 1.77 |
| 30 | 33.63 | 2.66 | 1.24 |
| 31 | 34.50 | 2.60 | 1.26 |
| 32 | 35.78 | 2.51 | 1.04 |
| 33 | 37.39 | 2.41 | 2.00 |

### Example 5—Maleate Form A

Maleate Form A was obtained via slurry of Freeform Form A and equal molar maleic acid in EtOAc at RT for 3 days and drying at RT under vacuum for 8 hrs. The XRPD pattern is shown in FIG. 6A. The TGA/DSC curves, shown in FIG. 6B, showed a weight loss of 1.33% up to 150° C. and two endotherms at 53.2° C. and 168.4° C. (peak temperature). ¹H NMR result showed that the molar ratio of maleic acid/freeform was 0.9 and negligible solvent EtOAc was detected. The purity of Maleate Form A was determined to be 99.37 area % by UPLC.

A heating experiment was performed to study the thermal signal around 53° C. XRPD results in FIG. 6C show that no form change was observed after heating Maleate Form A to 70° C., cooling to RT and exposure to ambient conditions. TGA/DSC results of the heated sample in FIG. 6D showed a weight loss of 3.27% up to 150° C. and two endotherms 53.5° C. and 168.4° C. (peak temperature).

TABLE 6

| #Peak | Pos. [°2θ] | d-spacing [Å] | Rel. Int. [%] |
|---|---|---|---|
| 1 | 9.84 | 8.99 | 7.47 |
| 2 | 11.31 | 7.83 | 31.96 |
| 3 | 13.04 | 6.79 | 12.53 |
| 4 | 13.30 | 6.66 | 10.68 |
| 5 | 14.19 | 6.24 | 100.00 |
| 6 | 15.58 | 5.69 | 67.08 |
| 7 | 15.78 | 5.62 | 29.39 |
| 8 | 16.44 | 5.39 | 52.19 |
| 9 | 17.44 | 5.09 | 14.76 |
| 10 | 18.51 | 4.79 | 13.48 |
| 11 | 19.11 | 4.65 | 50.53 |
| 12 | 19.90 | 4.46 | 75.66 |
| 13 | 21.24 | 4.18 | 4.29 |
| 14 | 22.14 | 4.01 | 61.26 |
| 15 | 22.90 | 3.88 | 19.08 |
| 16 | 23.83 | 3.73 | 12.70 |
| 17 | 24.25 | 3.67 | 37.16 |
| 18 | 24.81 | 3.59 | 2.37 |
| 19 | 25.39 | 3.51 | 41.99 |
| 20 | 25.94 | 3.43 | 5.93 |
| 21 | 26.73 | 3.33 | 4.56 |
| 22 | 27.16 | 3.28 | 1.99 |
| 23 | 28.12 | 3.17 | 5.76 |
| 24 | 28.49 | 3.13 | 15.64 |
| 25 | 29.52 | 3.03 | 15.06 |
| 26 | 29.94 | 2.98 | 8.80 |
| 27 | 30.24 | 2.96 | 7.31 |
| 28 | 30.64 | 2.92 | 6.09 |
| 29 | 31.26 | 2.86 | 1.73 |
| 30 | 31.92 | 2.80 | 7.13 |
| 31 | 32.36 | 2.77 | 3.84 |
| 32 | 34.11 | 2.63 | 3.15 |
| 33 | 36.66 | 2.45 | 2.95 |
| 34 | 37.39 | 2.41 | 3.03 |
| 35 | 38.85 | 2.32 | 2.50 |

### Example 6—HCl Form A

HCl Form A was obtained via slurry of Freeform Form A and equal molar acid in acetone at RT for 3 days and drying at RT under vacuum for 8 hrs. TGA/DSC curves were shown in FIG. 7A. TGA result, as shown in FIG. 7B, showed a weight loss of 8.04% up to 150° C. DSC result showed two endotherms at 80.1° C. and 199.1° C. (peak temperature). ¹H NMR showed that no obvious residual solvent acetone was detected. The stoichiometric ratio of Cl⁻/freeform was 1.0 and the purity was determined to be 99.81 area %. To study the thermal signal around 80° C., heating experiment was performed on HCl Form A. XRPD results in FIG. 7C showed that no form change was observed after heating to 100° C., cooling to RT and exposure to ambient conditions. TGA/DSC results of the heated sample in FIG. 7D showed a weight loss of 9.50% up to 150° C. and two endotherms at 67.3° C. and 198.7° C. (peak temperature). Based on the result, HCl Form A may be a hydrate.

TABLE 7

| #Peak | Pos. [°2θ] | d-spacing [Å] | Rel. Int. [%] |
|---|---|---|---|
| 1 | 11.35 | 7.79 | 30.93 |
| 2 | 11.73 | 7.55 | 17.70 |
| 3 | 13.25 | 6.68 | 20.78 |
| 4 | 16.60 | 5.34 | 100.00 |
| 5 | 17.03 | 5.21 | 39.12 |
| 6 | 17.74 | 5.00 | 17.13 |
| 7 | 18.50 | 4.80 | 64.58 |
| 8 | 18.75 | 4.73 | 57.82 |
| 9 | 20.86 | 4.26 | 28.65 |
| 10 | 21.44 | 4.14 | 3.40 |
| 11 | 22.07 | 4.03 | 7.09 |
| 12 | 22.74 | 3.91 | 8.12 |
| 13 | 23.52 | 3.78 | 12.14 |
| 14 | 23.99 | 3.71 | 8.56 |
| 15 | 24.87 | 3.58 | 24.01 |
| 16 | 25.77 | 3.46 | 21.58 |
| 17 | 26.01 | 3.43 | 17.79 |
| 18 | 26.25 | 3.39 | 32.78 |
| 19 | 26.68 | 3.34 | 35.63 |
| 20 | 27.63 | 3.23 | 14.54 |
| 21 | 28.10 | 3.18 | 17.88 |
| 22 | 28.52 | 3.13 | 11.35 |
| 23 | 29.57 | 3.02 | 5.99 |
| 24 | 30.32 | 2.95 | 6.68 |
| 25 | 31.01 | 2.88 | 2.66 |
| 26 | 31.45 | 2.84 | 3.09 |
| 27 | 33.29 | 2.69 | 4.96 |
| 28 | 33.87 | 2.65 | 3.49 |
| 29 | 34.38 | 2.61 | 5.54 |
| 30 | 35.20 | 2.55 | 3.81 |
| 31 | 36.89 | 2.44 | 1.97 |
| 32 | 37.93 | 2.37 | 2.53 |
| 33 | 38.63 | 2.33 | 6.35 |
| 34 | 39.60 | 2.28 | 1.84 |

### Example 7—HCl Form B

HCl Form B was obtained via slurry of Freeform Form A and equal molar acid in EtOAc at RT for 3 days and drying at RT under vacuum for 8 hrs. XRPD results are shown in FIG. 8A. TGA/DSC results, as shown in FIG. 8B, show a weight loss of 11.58% up to 100° C. and three endotherms at 68.5° C., 81.0° C. and 197.2° C. (peak temperature). ¹H NMR results showed that no obvious residual solvent EtOAc was detected. The stoichiometric ratio of Cl⁻/freeform was 1.0 and the purity was determined to be 99.08 area %. After heating to 100° C., cooling to RT and exposure to ambient conditions, a mixture of HCl salt Type A+B was obtained, as shown in FIG. 8C.

TABLE 8

| #Peak | Pos. [°2θ] | d-spacing [Å] | Rel. Int. [%] |
|---|---|---|---|
| 1 | 10.68 | 8.29 | 33.77 |
| 2 | 11.46 | 7.72 | 15.29 |
| 3 | 12.36 | 7.16 | 16.71 |
| 4 | 12.60 | 7.03 | 24.81 |
| 5 | 13.42 | 6.60 | 48.97 |
| 6 | 14.70 | 6.03 | 11.98 |
| 7 | 15.45 | 5.74 | 13.49 |
| 8 | 16.03 | 5.53 | 13.60 |
| 9 | 16.84 | 5.26 | 100.00 |
| 10 | 17.47 | 5.08 | 8.59 |
| 11 | 18.18 | 4.88 | 59.21 |
| 12 | 18.48 | 4.80 | 19.99 |
| 13 | 19.55 | 4.54 | 36.25 |
| 14 | 19.79 | 4.49 | 69.39 |
| 15 | 20.25 | 4.39 | 14.94 |
| 16 | 21.38 | 4.16 | 4.11 |
| 17 | 21.82 | 4.07 | 8.65 |
| 18 | 22.54 | 3.94 | 9.49 |
| 19 | 23.12 | 3.85 | 4.70 |
| 20 | 24.74 | 3.60 | 32.66 |
| 21 | 25.75 | 3.46 | 17.08 |
| 22 | 26.07 | 3.42 | 8.24 |
| 23 | 26.85 | 3.32 | 19.93 |
| 24 | 27.19 | 3.28 | 17.73 |
| 25 | 27.73 | 3.22 | 23.88 |
| 26 | 28.37 | 3.15 | 33.10 |
| 27 | 29.60 | 3.02 | 9.04 |
| 28 | 30.36 | 2.94 | 5.13 |
| 29 | 31.81 | 2.81 | 2.23 |
| 30 | 33.61 | 2.67 | 3.83 |
| 31 | 34.12 | 2.63 | 3.05 |
| 32 | 37.50 | 2.40 | 2.46 |

### Example 8—HCl Form C

HCl Form C was obtained via procedures as follows. After stirring the mixture of Freeform Form A and equal molar acid in EtOH at RT for 3 days for 2 days, at 5° C. for 2 days and at −20° C. for 1 day, clear solution was obtained. Anti-solvent n-heptane was added. A clear solution was still observed. To obtain solids, the sample was transferred to stir at 5° C. and −20° C., followed by evaporation at RT. XRPD results are shown in FIG. 9A. TGA/DSC curves, as shown in FIG. 9B, showed a weight loss of 16.74% up to 110° C. and DSC showed three endotherms at 99.6° C., 145.7° C. and 195.8° C. (peak temperature). ¹H NMR result showed that the molar ratio of residual solvent EtOH/API was 0.04 (0.6 wt %). The stoichiometric ratio of Cl⁻/freeform was 0.8 and the purity was determined to be 99.29 area %.

TABLE 9

| #Peak | Pos. [°2θ] | d-spacing [Å] | Rel. Int. [%] |
|---|---|---|---|
| 1 | 6.08 | 14.54 | 2.08 |
| 2 | 8.96 | 9.87 | 40.33 |
| 3 | 10.39 | 8.52 | 24.76 |
| 4 | 12.41 | 7.13 | 29.61 |
| 5 | 14.84 | 5.97 | 1.36 |
| 6 | 16.87 | 5.26 | 32.28 |
| 7 | 17.14 | 5.17 | 14.18 |
| 8 | 17.94 | 4.95 | 100.00 |
| 9 | 18.26 | 4.86 | 2.62 |
| 10 | 19.19 | 4.62 | 5.86 |
| 11 | 20.13 | 4.41 | 5.73 |
| 12 | 22.32 | 3.98 | 3.47 |
| 13 | 22.76 | 3.91 | 5.92 |
| 14 | 23.15 | 3.84 | 0.90 |
| 15 | 24.41 | 3.65 | 7.88 |
| 16 | 24.92 | 3.57 | 1.69 |
| 17 | 25.48 | 3.50 | 3.80 |
| 18 | 25.85 | 3.45 | 4.66 |
| 19 | 26.13 | 3.41 | 30.30 |
| 20 | 28.22 | 3.16 | 6.79 |
| 21 | 28.80 | 3.10 | 1.78 |
| 22 | 30.00 | 2.98 | 2.56 |
| 23 | 32.40 | 2.76 | 0.88 |
| 24 | 33.30 | 2.69 | 0.27 |
| 25 | 34.05 | 2.63 | 7.99 |
| 26 | 34.62 | 2.59 | 2.25 |
| 27 | 36.27 | 2.48 | 4.26 |
| 28 | 36.96 | 2.43 | 0.37 |
| 29 | 37.71 | 2.39 | 0.95 |
| 30 | 38.88 | 2.32 | 2.03 |

### Example 9—Phosphate Form A

Phosphate Form A was obtained via slurry of Freeform Form A and equal molar H₃PO₄ in acetone at RT for 3 days and drying at RT under vacuum for 8 hrs. XRPD results are shown in FIG. 10A. TGA/DSC curves, as shown in FIG. 10B, showed a weight loss of 9.03% up to 150° C. and two endotherms at 73.8° C. and 104.8° C. (peak temperature). ¹H NMR result showed that the molar ratio of acetone/API was 0.5 (7.1 wt %). The stoichiometric ratio of PO₄³⁻/freeform was 1.1 by UPLC/IC. The purity of Phosphate Form A was determined to be 98.84 area % by UPLC.

TABLE 10

| #Peak | Pos. [°2θ] | d-spacing [Å] | Rel. Int. [%] |
|---|---|---|---|
| 1 | 3.94 | 22.41 | 100.00 |
| 2 | 4.86 | 18.18 | 17.21 |
| 3 | 9.75 | 9.08 | 2.80 |
| 4 | 11.67 | 7.58 | 12.90 |
| 5 | 12.32 | 7.18 | 5.49 |
| 6 | 13.23 | 6.69 | 8.07 |
| 7 | 14.78 | 5.99 | 9.19 |
| 8 | 15.82 | 5.60 | 26.39 |
| 9 | 17.48 | 5.07 | 22.39 |
| 10 | 17.93 | 4.95 | 15.59 |
| 11 | 18.49 | 4.80 | 14.61 |
| 12 | 19.50 | 4.55 | 26.71 |
| 13 | 20.72 | 4.29 | 5.74 |
| 14 | 22.44 | 3.96 | 19.60 |
| 15 | 23.68 | 3.76 | 9.26 |
| 16 | 24.58 | 3.62 | 9.66 |
| 17 | 25.37 | 3.51 | 8.65 |
| 18 | 26.21 | 3.40 | 8.05 |
| 19 | 28.94 | 3.08 | 5.19 |

### Example 10—Tartrate Form A

Tartrate Form A was obtained via slurry of Freeform Form A and equal molar L-tartaric acid in acetone at RT for 3 days and drying at RT under vacuum for 8 hrs. XRPD pattern is shown in FIG. 11A. TGA/DSC curves were shown in FIG. 11B. TGA result showed a weight loss of 6.54% up to 160° C. DSC result showed two endotherms at 67.7° C. and 96.6° C. (peak temperature). ¹H NMR showed that the molar ratio of L-tartaric acid/freeform was 1.2 and residual solvent acetone/API was 0.2 (2.9 wt %). The purity of Tartrate Form A was determined to be 98.62 area % by UPLC.

TABLE 11

| #Peak | Pos. [°2θ] | d-spacing [Å] | Rel. Int. [%] |
|---|---|---|---|
| 1 | 5.01 | 17.63 | 100.00 |
| 2 | 7.02 | 12.59 | 44.26 |
| 3 | 12.23 | 7.24 | 18.02 |
| 4 | 14.08 | 6.29 | 80.89 |
| 5 | 14.91 | 5.94 | 52.20 |
| 6 | 17.28 | 5.13 | 50.60 |
| 7 | 18.36 | 4.83 | 45.36 |
| 8 | 19.71 | 4.50 | 26.94 |
| 9 | 20.17 | 4.40 | 23.48 |
| 10 | 21.20 | 4.19 | 32.44 |
| 11 | 22.69 | 3.92 | 19.84 |
| 12 | 24.34 | 3.66 | 20.64 |
| 13 | 25.33 | 3.52 | 24.93 |
| 14 | 26.69 | 3.34 | 17.42 |

### Example 11—Fumarate Form A

Fumarate Form A was obtained via slurry of Freeform Form A and equal molar fumaric acid in EtOAc at RT for 3 days and drying at RT under vacuum for 8 hrs. XRPD pattern is shown in FIG. 12A. TGA/DSC curves in FIG. 12B showed a weight loss of 1.50% up to 150° C. and two endotherms at 160.2° C. and 180.7° C. (peak temperature). ¹H NMR spectrum showed the molar ratio of acid/API was 1.2 and residual solvent EtOAc/API was 0.04 (0.7 wt %). The purity of Fumarate Form A was determined to be 98.85 area % by UPLC.

TABLE 12

| #Peak | Pos. [°2θ] | d-spacing [Å] | Rel. Int. [%] |
|---|---|---|---|
| 1 | 11.14 | 7.94 | 6.29 |
| 2 | 12.73 | 6.95 | 100.00 |
| 3 | 13.02 | 6.80 | 12.07 |
| 4 | 13.30 | 6.66 | 10.74 |
| 5 | 14.10 | 6.28 | 35.46 |
| 6 | 15.09 | 5.87 | 41.38 |
| 7 | 15.35 | 5.77 | 15.15 |
| 8 | 16.27 | 5.45 | 3.28 |
| 9 | 17.59 | 5.04 | 8.29 |
| 10 | 18.40 | 4.82 | 64.28 |
| 11 | 18.81 | 4.72 | 60.85 |
| 12 | 19.39 | 4.58 | 26.58 |
| 13 | 19.74 | 4.50 | 53.42 |
| 14 | 20.53 | 4.33 | 26.05 |
| 15 | 22.09 | 4.02 | 29.44 |
| 16 | 22.29 | 3.99 | 19.82 |
| 17 | 23.06 | 3.86 | 79.62 |
| 18 | 23.75 | 3.75 | 10.67 |
| 19 | 24.26 | 3.67 | 5.32 |
| 20 | 25.00 | 3.56 | 3.56 |
| 21 | 26.18 | 3.40 | 16.33 |
| 22 | 27.08 | 3.29 | 8.00 |
| 23 | 28.28 | 3.16 | 10.68 |
| 24 | 28.81 | 3.10 | 16.81 |
| 25 | 30.78 | 2.90 | 6.56 |
| 26 | 33.75 | 2.66 | 3.72 |
| 27 | 35.81 | 2.51 | 2.40 |
| 28 | 37.93 | 2.37 | 1.82 |
| 29 | 39.29 | 2.29 | 2.53 |

### Example 12—Naphthalene-1,5-disulfonate Form A

Naphthalene-1,5-disulfonate Form A was obtained via slurry of Freeform Form A and equal molar naphthalene-1,5-disulfonic acid in EtOAc at RT for 3 days and drying at RT under vacuum for 8 hrs. XRPD results are shown in FIG. 13A and TGA/DSC results are shown in FIG. 13B. TGA result showed a weight loss of 10.03% up to 180° C. DSC result showed three endotherms at 94.2° C., 136.7° C. and 195.0° C. (peak temperature). ¹H NMR showed that the molar ratio of acid/freeform was 1.8 and residual solvent EtOAc/API was 0.07 (0.7 wt %). The purity of Naphthalene-1,5-disulfonate Form A was determined to be 94.51 area % by UPLC.

TABLE 13

| #Peak | Pos. [°2θ] | d-spacing [Å] | Rel. Int. [%] |
|---|---|---|---|
| 1 | 11.76 | 7.52 | 27.34 |
| 2 | 12.43 | 7.12 | 17.08 |
| 3 | 13.87 | 6.38 | 100.00 |
| 4 | 14.53 | 6.10 | 46.37 |
| 5 | 15.16 | 5.84 | 48.04 |
| 6 | 16.39 | 5.41 | 85.84 |
| 7 | 18.10 | 4.90 | 82.40 |
| 8 | 19.51 | 4.55 | 55.08 |
| 9 | 20.07 | 4.43 | 36.50 |
| 10 | 20.73 | 4.29 | 44.94 |
| 11 | 21.99 | 4.04 | 13.12 |
| 12 | 23.51 | 3.78 | 40.45 |
| 13 | 23.94 | 3.72 | 37.80 |
| 14 | 25.16 | 3.54 | 32.30 |
| 15 | 25.57 | 3.48 | 33.29 |

### Example 13—Oxalate Form A

Oxalate Form A was obtained via slurry of Freeform Form A and equal molar oxalic acid in EtOAc at RT for 3 days and drying at RT under vacuum for 8 hrs. XRPD results are shown in FIG. 14A. TGA/DSC curves, as shown in FIG. 14B, show a weight loss of 8.26% up to 100° C. and two endotherms at 88.4° C. and 97.1° C. (peak temperature). ¹H NMR showed that the molar ratio of residual solvent EtOAc/API was 0.1 (2.2 wt %). The stoichiometric ratio of C₂O₄²⁻/freeform was 0.6 by UPLC/IC. The purity of Oxalate Form A was determined to be 98.24 area % by UPLC.

TABLE 14

| #Peak | Pos. [°2θ] | d-spacing [Å] | Rel. Int. [%] |
|---|---|---|---|
| 1 | 6.04 | 14.62 | 8.69 |
| 2 | 10.75 | 8.23 | 1.93 |
| 3 | 11.96 | 7.40 | 47.29 |
| 4 | 12.51 | 7.07 | 13.45 |
| 5 | 13.57 | 6.53 | 4.72 |
| 6 | 15.94 | 5.56 | 54.54 |
| 7 | 16.24 | 5.46 | 8.34 |
| 8 | 17.06 | 5.20 | 15.33 |
| 9 | 18.30 | 4.85 | 100.00 |
| 10 | 19.38 | 4.58 | 21.29 |
| 11 | 20.02 | 4.44 | 29.50 |
| 12 | 20.86 | 4.26 | 8.38 |
| 13 | 21.55 | 4.12 | 6.88 |
| 14 | 21.99 | 4.04 | 21.05 |
| 15 | 23.57 | 3.77 | 2.38 |
| 16 | 24.03 | 3.70 | 11.51 |
| 17 | 24.21 | 3.68 | 15.36 |
| 18 | 24.81 | 3.59 | 6.20 |
| 19 | 25.11 | 3.55 | 8.69 |
| 20 | 25.69 | 3.47 | 13.07 |
| 21 | 26.56 | 3.36 | 19.44 |
| 22 | 27.24 | 3.27 | 7.91 |
| 23 | 27.61 | 3.23 | 2.83 |
| 24 | 28.05 | 3.18 | 4.22 |
| 25 | 29.21 | 3.06 | 9.61 |
| 26 | 30.61 | 2.92 | 4.41 |
| 27 | 31.37 | 2.85 | 5.46 |
| 28 | 31.81 | 2.81 | 11.22 |
| 29 | 32.29 | 2.77 | 3.07 |
| 30 | 32.76 | 2.73 | 2.59 |
| 31 | 33.58 | 2.67 | 2.64 |
| 32 | 34.12 | 2.63 | 3.08 |
| 33 | 34.42 | 2.61 | 3.77 |
| 34 | 35.78 | 2.51 | 1.64 |
| 35 | 37.10 | 2.42 | 5.01 |
| 36 | 38.42 | 2.34 | 2.90 |

### Example 14—HBr Form A

HBr Form A was obtained via slurry of Freeform Form A and equal molar HBr in acetone at RT for 3 days and drying at RT under vacuum for 8 hrs. XRPD results are shown in FIG. 15A. TGA/DSC curves, as shown in FIG. 15B, showed a weight loss of 4.68% up to 80° C. and two endotherms at 92.2° C. and 203.6° C. (peak temperature). ¹H NMR spectrum showed no obvious residual solvent acetone was detected. The stoichiometric ratio of Br/freeform was 1.0 by UPLC/IC. The purity was determined to be 99.80 area % by UPLC.

To study the thermal signal around 92° C., HBr Form A was heated to 100° C., cooled to RT and exposed to ambient conditions. XRPD results in FIG. 15C showed that no form change was observed after heating. Further characterization was performed on the heated sample. TGA/DSC results (FIG. 15D) showed a weight loss of 9.69% up to 150° C. and three endotherms at 67.6° C., 82.2° C. and 203.2° C. (peak temperature). Based on the result, HBr Form A was speculated to be a hydrate. The endotherms before 100° C. might be caused by dehydration.

TABLE 15

| #Peak | Pos. [°2θ] | d-spacing [Å] | Rel. Int. [%] |
|---|---|---|---|
| 1 | 11.65 | 7.60 | 33.51 |
| 2 | 14.41 | 6.15 | 3.29 |
| 3 | 16.36 | 5.42 | 100.00 |
| 4 | 17.25 | 5.14 | 43.25 |
| 5 | 17.88 | 4.96 | 9.83 |
| 6 | 18.57 | 4.78 | 87.12 |
| 7 | 20.87 | 4.26 | 16.32 |
| 8 | 21.40 | 4.15 | 12.57 |
| 9 | 22.73 | 3.91 | 7.84 |
| 10 | 23.01 | 3.86 | 12.97 |
| 11 | 23.41 | 3.80 | 39.24 |
| 12 | 24.11 | 3.69 | 4.99 |
| 13 | 24.86 | 3.58 | 29.16 |
| 14 | 25.87 | 3.44 | 33.68 |
| 15 | 26.23 | 3.40 | 79.65 |
| 16 | 27.56 | 3.24 | 27.19 |
| 17 | 28.32 | 3.15 | 6.04 |
| 18 | 28.78 | 3.10 | 3.21 |
| 19 | 29.23 | 3.06 | 9.29 |
| 20 | 30.32 | 2.95 | 14.34 |
| 21 | 31.15 | 2.87 | 5.28 |
| 22 | 33.09 | 2.71 | 6.49 |
| 23 | 34.35 | 2.61 | 4.29 |
| 24 | 34.97 | 2.57 | 4.87 |
| 25 | 35.51 | 2.53 | 4.53 |
| 26 | 36.78 | 2.44 | 3.33 |
| 27 | 37.39 | 2.41 | 5.31 |
| 28 | 38.36 | 2.35 | 1.91 |
| 29 | 39.19 | 2.30 | 3.40 |

1.-73. (canceled)

74. A composition comprising a salt of mesembranol selected from the group consisting of a hydrochloride salt of mesembranol, a sulfate salt of mesembranol, a malate salt of mesembranol, a maleate salt of mesembranol, a gentisate salt of mesembranol, a phosphate salt of mesembranol, a tartrate salt of mesembranol, a fumarate salt of mesembranol, a naphthalene-1,5-disulfonate salt of mesembranol, an oxalate salt of mesembranol and a hydrobromide salt of mesembranol.

75. The composition of claim 74, comprising a salt of (−) mesembranol.

76. The composition of claim 75, wherein the salt of (−) mesembranol is a hydrochloride salt of (−) mesembranol.

77. The composition of claim 76, wherein the hydrochloride salt of (−) mesembranol is characterized by an X-ray powder diffraction (XRPD) pattern comprising 2-theta peaks (2-theta±0.2) at 16.6°, 17.0°, 18.5°, 18.8°, and 26.7°.

78. The composition of claim 76, wherein the hydrochloride salt of (−) mesembranol is characterized by a sample weight loss of up to about 8.5% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |
| | |
|---|---|
| Purge gas | N₂. |

79. The composition of claim 76, wherein the hydrochloride salt of (−) mesembranol is characterized by a DSC having an endotherm at about 197° C. to about 202° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

80. The composition of claim 76, wherein the hydrochloride salt of (−) mesembranol is characterized by the following:
a. an X-ray powder diffraction (XRPD) pattern comprising 2-theta peaks (2-theta±0.2) at 16.6°, 17.0°, 18.5°, 18.8°, and 26.7°;
b. a sample weight loss of up to about 8.5% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |
| | |
|---|---|
| Purge gas | N₂; |

and
c. a DSC having an endotherm at about 197° C. to about 202° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

81. The composition of claim 76, wherein the hydrochloride salt of (−) mesembranol is solid Form A of the hydrochloride salt of (−) mesembranol.

82. The composition of claim 76, wherein the hydrochloride salt of (−) mesembranol is characterized by an X-ray powder diffraction (XRPD) pattern comprising 2-theta peaks (2-theta±0.2) at 16.6°, 17.0°, 18.5°, 18.8°, and 26.7°.

83. The composition of claim 76, wherein the hydrochloride salt of (−) mesembranol is characterized by the following:
a. an X-ray powder diffraction (XRPD) pattern comprising 2-theta peaks (2-theta±0.2) at 16.6°, 17.0°, 18.5°, 18.8°, and 26.7°;
b. a sample weight loss of up to about 8.5% upon heating to 150° C. measured by TGA according to the following parameters:

| Method | Ramp | |
|---|---|---|
| Sample pan | Aluminum, open | |
| Temperature | RT-350 | °C |
| Heating rate | 10 | °C/min |
| | |
|---|---|
| Purge gas | N₂; |

and
c. a DSC having an endotherm at about 197° C. to about 202° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

84. The composition of claim 76, wherein the hydrochloride salt of (−) mesembranol is solid Form B of the hydrochloride salt of (−) mesembranol.

85. The composition of claim 76, wherein the hydrochloride salt of (−) mesembranol is characterized by an X-ray powder diffraction (XRPD) pattern comprising 2-theta peaks (2-theta±0.2) at 13.4°, 16.8°, 18.2°, 19.6°, and 19.8°.

86. The composition of claim 76, wherein the hydrochloride salt of (−) mesembranol is characterized by a sample weight loss of 8.5%-12% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min. |

87. The composition of claim 76, wherein the hydrochloride salt of (−) mesembranol is characterized by a DSC having an endotherm at about 195° C. to about 200° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

88. The composition of claim 76, wherein the hydrochloride salt of (−) mesembranol is characterized by the following:
d. an X-ray powder diffraction (XRPD) pattern comprising 2-theta peaks (2-theta±0.2) at 13.4°, 16.8°, 18.2°, 19.6°, and 19.8°;
e. a sample weight loss of up to 12% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min; |

and
f. a DSC having an endotherm at about 195° C. to about 200° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

89. The composition of claim 76, wherein the hydrochloride salt of (−) mesembranol is solid Form C of the hydrochloride salt of (−) mesembranol.

90. The composition claim 76, wherein the hydrochloride salt of (−) mesembranol is characterized by an X-ray powder diffraction (XRPD) pattern comprising 2-theta peaks (2-theta±0.2) at 9.0°, 12.4°, 16.9°, 17.9°, and 26.1°.

91. The composition of claim 76, wherein the hydrochloride salt of (−) mesembranol is characterized by a sample weight loss of 12%-17% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |
| | |
|---|---|
| Purge gas | N₂. |

92. The composition of claim 76, wherein the hydrochloride salt of (−) mesembranol is characterized by a DSC having an endotherm at about 193° C. to about 197° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

93. The composition of claim 76, wherein the hydrochloride salt of (−) mesembranol is characterized by
g. an X-ray powder diffraction (XRPD) pattern comprising 2-theta peaks (2-theta±0.2) at 9.0°, 12.4°, 16.9°, 17.9°, and 26.1°;
h. a sample weight loss of 12%-17% upon heating to 150° C. measured by TGA according to the following parameters:

| Parameters | TGA | |
|---|---|---|
| Method | Ramp | |
| Sample pan | Aluminum, open | |
| Temperature | RT-350 | °C |
| Heating rate | 10 | °C/min |
| | |
|---|---|
| Purge gas | N₂. |

i. a DSC having an endotherm at about 193° C. to about 197° C. (peak temperature), measured by DSC according to the following parameters:

| Parameters | DSC |
|---|---|
| Method | Ramp |
| Sample pan | Aluminum, crimped |
| Temperature | 25 °C-230 °C |
| Heating rate | 10 °C/min |
| Purge gas | N₂. |

94. The composition of claim 75, wherein the salt of mesembranol is a solid form of (−) mesembranol selected from the group consisting of:
j. Sulfate Form A having an X-ray powder diffraction (XRPD) pattern comprising a 2-theta peak (2-theta±0.2) at 16.1°;
k. Malate Form A having an X-ray powder diffraction (XRPD) pattern comprising a 2-theta peak (2-theta±0.2) at 13.1°;
1. Malate Form B having an X-ray powder diffraction (XRPD) pattern comprising a 2-theta peak (2-theta±0.2) at 20.2;
m. Gentisate Form A having an X-ray powder diffraction (XRPD) pattern comprising a 2-theta peak (2-theta±0.2) at 16.6°;
n. Malcate Form A having an X-ray powder diffraction (XRPD) pattern comprising a 2-theta peak (2-theta±0.2) at 14.2°;
0. HCl Form A having an X-ray powder diffraction (XRPD) pattern comprising a 2-theta peak (2-theta±0.2) at 18.5°;
p. HCl Form B having an X-ray powder diffraction (XRPD) pattern comprising a 2-theta peak (2-theta±0.2) at 19.8°;
q. HCl Form C having an X-ray powder diffraction (XRPD) pattern comprising a 2-theta peak (2-theta±0.2) at 9.0°;
r. Phosphate Form A having an X-ray powder diffraction (XRPD) pattern comprising a 2-theta peak (2-theta±0.2) at 3.9°;
s. Tartrate Form A having an X-ray powder diffraction (XRPD) pattern comprising a 2-theta peak (2-theta±0.2) at 5.0°;
t. Fumarate Form A having an X-ray powder diffraction (XRPD) pattern comprising a 2-theta peak (2-theta±0.2) at 12.7°;
u. Napthalene-1,5-disulfonate Form A having an X-ray powder diffraction (XRPD) pattern comprising a 2-theta peak (2-theta±0.2) at 18.1°;
v. Oxalate Form A having an X-ray powder diffraction (XRPD) pattern comprising a 2-theta peak (2-theta±0.2) at 15.9°; and
w. HBr Form A having an X-ray powder diffraction (XRPD) pattern comprising a 2-theta peak (2-theta±0.2) at 16.4°.

95. A pharmaceutical composition comprising the salt of (−) mesembranol of claim 75, and a pharmaceutically acceptable excipient.

96. A method of treating a mental health disorder, comprising administering to a mammal in need thereof an effective amount the pharmaceutical composition of claim 95.
